SEQUENCE LISTING (498 sequences)

SEQ ID NO: 1 - DNA, 103 bp, synthetic construct
  source 1..103: mol_type = other DNA
gcgcgtcctggattcgcggaaacgcgtacatccagctgacgagtcccaaataggacgaaacgcgcagtgataccagcatcgtcttgatgcccttggcagcact

SEQ ID NO: 2 - DNA, 105 bp, synthetic construct
  source 1..105: mol_type = other DNA
gcgcgtcctggattcgcggaaacgcgtacatccagctgacgagtcccaaataggacgaaacgagaataccagcatcgtcttgatgcccttggaagtctcgcgcgc

SEQ ID NO: 3 - DNA, 103 bp, synthetic construct
  source 1..103: mol_type = other DNA
gcgcgtcctgcggaataccagcatcgtcttgatgcccttggaagtccggattcgcggaaacgcgtacatccagctgacgagtcccaaataggacgaaacgcgc

SEQ ID NO: 4 - DNA, 105 bp, synthetic construct
  source 1..105: mol_type = other DNA
gcgcgtcctggattcgcggaaacgcgtacatccagctgacgagtcccaaataggccggaataccagcatcgtcttgatgcccttggaagtccggacgaaacgcgc

SEQ ID NO: 5 - DNA, 105 bp, synthetic construct
  source 1..105: mol_type = other DNA
gcgcgtcctggattcgcggaaaccggaataccagcatcgtcttgatgcccttggaagtccggcgcgtacatccagctgacgagtcccaaataggacgaaacgcgc

SEQ ID NO: 6 - DNA, 103 bp, synthetic construct
  source 1..103: mol_type = other DNA
gcgcgtcctggattcgcggaaacgcgtacatccagctgacgagtcccaaataggacgaaacgagtgataccagcatcgtcttgatgcccttggcagcactcgc

SEQ ID NO: 7 - DNA, 105 bp, synthetic construct
  source 1..105: mol_type = other DNA
gcgcgtcctggattcgcggaaacgcgtacatccagctgacgagtcccaaataggacgaaacgcgagaataccagcatcgtcttgatgcccttggaagtctcgcgc

SEQ ID NO: 8 - DNA, 103 bp, synthetic construct
  source 1..103: mol_type = other DNA
gcgcgtcctggattcgcggaaacggaataccagcatcgtcttgatgcccttggaagtccgcgcgtacatccagctgacgagtcccaaataggacgaaacgcgc

SEQ ID NO: 9 - DNA, 105 bp, synthetic construct
  source 1..105: mol_type = other DNA
gcgcgtcctggattcgcggaaacgcgtacatccagctgacgagtcccaaataggacgaccggaataccagcatcgtcttgatgcccttggaagtccggaacgcgc

SEQ ID NO: 10 - DNA, 105 bp, synthetic construct
  source 1..105: mol_type = other DNA
gcgcgtcctggacgagaataccagcatcgtcttgatgcccttggaagtctcgttcgcggaaacgcgtacatccagctgacgagtcccaaataggacgaaacgcgc

SEQ ID NO: 11 - DNA, 103 bp, synthetic construct
  source 1..103: mol_type = other DNA
gcgcgtcctggattcgcggaaacgcgtacatccagctgacgagtcccggaataccagcatcgtcttgatgcccttggaagtccgcaaataggacgaaacgcgc

SEQ ID NO: 12 - DNA, 101 bp, synthetic construct
  source 1..101: mol_type = other DNA
gcgcgtcctggattcgcggaaacagaataccagcatcgtcttgatgcccttggaagtctgcgtacatccagctgacgagtcccaaataggacgaaacgcgc

SEQ ID NO: 13 - DNA, 101 bp, synthetic construct
  source 1..101: mol_type = other DNA
gcgcgtcctggattcgcggaaacgcgtacatccagctgacgagtcccaaataggacgaaacgagaataccagcatcgtcttgatgcccttggaagtctcgc

SEQ ID NO: 14 - DNA, 101 bp, synthetic construct
  source 1..101: mol_type = other DNA
gcgcgtcctggattcgcggaaacgcgtacatccagctgacgagtcccaaataggacgaaacggaataccagcatcgtcttgatgcccttggaagtccgcgc

SEQ ID NO: 15 - DNA, 103 bp, synthetic construct
  source 1..103: mol_type = other DNA
gcgcgtcctggattcgcggcggaataccagcatcgtcttgatgcccttggaagtccgaaacgcgtacatccagctgacgagtcccaaataggacgaaacgcgc

SEQ ID NO: 16 - DNA, 103 bp, synthetic construct
  source 1..103: mol_type = other DNA
gcgcgtcctggatgagaataccagcatcgtcttgatgcccttggaagtctctcgcggaaacgcgtacatccagctgacgagtcccaaataggacgaaacgcgc

SEQ ID NO: 17 - DNA, 103 bp, synthetic construct
  source 1..103: mol_type = other DNA
gcgcgtcctggcggaataccagcatcgtcttgatgcccttggaagtccgattcgcggaaacgcgtacatccagctgacgagtcccaaataggacgaaacgcgc

SEQ ID NO: 18 - DNA, 103 bp, synthetic construct
  source 1..103: mol_type = other DNA
gcgcgtcctggattcgcggaaacgcgtacatccagctgacgagtcccaaagtgataccagcatcgtcttgatgcccttggcagcactataggacgaaacgcgc

SEQ ID NO: 19 - DNA, 101 bp, synthetic construct
  source 1..101: mol_type = other DNA
gcgcgtcctggattcgcggaaacgcgtacatccagctgacgggaataccagcatcgtcttgatgcccttggaagtccagtcccaaataggacaaaacgcgc

SEQ ID NO: 20 - DNA, 103 bp, synthetic construct
  source 1..103: mol_type = other DNA
gcgcgtcctggattcgcggaaacgcgtacatcggaataccagcatcgtcttgatgcccttggaagtccgccagctgacgagtcccaaataggacgaaacgcgc

SEQ ID NO: 21 - DNA, 103 bp, synthetic construct
  source 1..103: mol_type = other DNA
gcgcgtcctggattcgcggaaacgcgtacatccagctgacgagtcccaaatagagtgataccagcatcgtcttgatgcccttggcagcactgacgaaacgcgc

SEQ ID NO: 22 - DNA, 103 bp, synthetic construct
  source 1..103: mol_type = other DNA
gcgcgtcctggattcgcggaaacgcgtacaagtgataccagcatcgtcttgatgcccttggcagcacttccagctgacgagtcccaaataggacgaaacgcgc

SEQ ID NO: 23 - DNA, 103 bp, synthetic construct
  source 1..103: mol_type = other DNA
gcgcgtcctggattcgcggaaacgcgtgagaataccagcatcgtcttgatgcccttggaagtctcacatccagctgacgagtcccaaataggacgaaacgcgc

SEQ ID NO: 24 - DNA, 103 bp, synthetic construct
  source 1..103: mol_type = other DNA
gcgcgtcctggattcgcggaaacgcgtacatccagctgacgagtcccaaataggacagtgataccagcatcgtcttgatgcccttggcagcactgaaacgcgc

SEQ ID NO: 25 - DNA, 105 bp, synthetic construct
  source 1..105: mol_type = other DNA
gcgcgtcccgagaataccagcatcgtcttgatgcccttggaagtctcgtggattcgcggaaacgcgtacatccagctgacgagtcccaaataggacgaaacgcgc

SEQ ID NO: 26 - DNA, 105 bp, synthetic construct
  source 1..105: mol_type = other DNA
gcgcgtcctggattcgcggaaacgcgtacatccagctgacgagtccgagaataccagcatcgtcttgatgcccttggaagtctcgccaaataggacgaaacgcgc

SEQ ID NO: 27 - DNA, 103 bp, synthetic construct
  source 1..103: mol_type = other DNA
gcgcgtcctggattcgcggaaacgcgtacagtgataccagcatcgtcttgatgcccttggcagcactatccagctgacgagtcccaaataggacgaaacgcgc

SEQ ID NO: 28 - DNA, 103 bp, synthetic construct
  source 1..103: mol_type = other DNA
gcgcgtcctggattcgcggaaacgcgtacatccagctgacgacggaataccagcatcgtcttgatgcccttggaagtccggtcccaaataggacgaaacgcgc

SEQ ID NO: 29 - DNA, 101 bp, synthetic construct
  source 1..101: mol_type = other DNA
gcgcgtcctggattcgcggaaacgggaataccagcatcgtcttgatgcccttggaagtcccgtacatccagctgacgagtcccaaataggacgaaacgcgc

SEQ ID NO: 30 - DNA, 103 bp, synthetic construct
  source 1..103: mol_type = other DNA
gcgcgtcctggattcgcggaaacgcgtacatccagctgacgagtcccaaataggacgaaacgagaataccagcatcgtcttgatgcccttggaagtctcgcgc

SEQ ID NO: 31 - DNA, 103 bp, synthetic construct
  source 1..103: mol_type = other DNA
gcgcgtccgagaataccagcatcgtcttgatgcccttggaagtctctggattcgcggaaacgcgtacatccagctgacgagtcccaaataggacgaaacgcgc

SEQ ID NO: 32 - DNA, 103 bp, synthetic construct
  source 1..103: mol_type = other DNA
gcgcgtcctggattcgcggaaaagtgataccagcatcgtcttgatgcccttggcagcactcgcgtacatccagctgacgagtcccaaataggacgaaacgcgc

SEQ ID NO: 33 - DNA, 105 bp, synthetic construct
  source 1..105: mol_type = other DNA
gcgcgtcctggattcgcggaaacgcgtacatccagctgacgagtcccaaatccggaataccagcatcgtcttgatgcccttggaagtccggaggacgaaacgcgc

SEQ ID NO: 34 - DNA, 103 bp, synthetic construct
  source 1..103: mol_type = other DNA
gcgcgtcctggattcgcggaaacgcgtacatccagctgacgagagaataccagcatcgtcttgatgcccttggaagtctcgtcccaaataggacgaaacgcgc

SEQ ID NO: 35 - DNA, 103 bp, synthetic construct
  source 1..103: mol_type = other DNA
gcgcgtcctggattcgcggaaacgcgtacatccagctgacgagtcccaaataggacgaaacggaataccagcatcgtcttgatgcccttggaagtccgcgcgc

SEQ ID NO: 36 - DNA, 105 bp, synthetic construct
  source 1..105: mol_type = other DNA
gcgcgtcctggattcgcggaaacgcgtacatccagctgacgagtcccaaataggcgagaataccagcatcgtcttgatgcccttggaagtctcgacgaaacgcgc

SEQ ID NO: 37 - DNA, 103 bp, synthetic construct
  source 1..103: mol_type = other DNA
gcgcgtcctggattcgccggaataccagcatcgtcttgatgcccttggaagtccgggaaacgcgtacatccagctgacgagtcccaaataggacgaaacgcgc

SEQ ID NO: 38 - DNA, 101 bp, synthetic construct
  source 1..101: mol_type = other DNA
gcgcgtcctggattcgcggaaacgcgtacatcagaataccagcatcgtcttgatgcccttggaagtctcagctgacgagtcccaaataggacgaaacgcgc

SEQ ID NO: 39 - DNA, 101 bp, synthetic construct
  source 1..101: mol_type = other DNA
gcgcgtcctggattcgcggaaacgcgtacatcggaataccagcatcgtcttgatgcccttggaagtcccagctgacgagtcccaaataggacgaaacgcgc

SEQ ID NO: 40 - DNA, 101 bp, synthetic construct
  source 1..101: mol_type = other DNA
gcgcgtcctggattcgcgagaataccagcatcgtcttgatgcccttggaagtctgaaacgcgtacatccagctgacgagtcccaaataggacgaaacgcgc

SEQ ID NO: 41 - DNA, 103 bp, synthetic construct
  source 1..103: mol_type = other DNA
gcgcgtcctggattcgcggaaacgcgagtgataccagcatcgtcttgatgcccttggcagcacttacatccagctgacgagtcccaaataggacgaaacgcgc

SEQ ID NO: 42 - DNA, 101 bp, synthetic construct
  source 1..101: mol_type = other DNA
gcgcgtcctggattcgcggaaacgcgtacatccaagaataccagcatcgtcttgatgcccttggaagtctgctgacgagtcccaaataggacgaaacgcgc

SEQ ID NO: 43 - DNA, 105 bp, synthetic construct
  source 1..105: mol_type = other DNA
gcgcgtcctcgagaataccagcatcgtcttgatgcccttggaagtctcgggattcgcggaaacgcgtacatccagctgacgagtcccaaataggacgaaacgcgc

SEQ ID NO: 44 - DNA, 103 bp, synthetic construct
  source 1..103: mol_type = other DNA
gcggagaataccagcatcgtcttgatgcccttggaagtctccgtcctggattcgcggaaacgcgtacatccagctgacgagtcccaaataggacgaaacgcgc

SEQ ID NO: 45 - DNA, 103 bp, synthetic construct
  source 1..103: mol_type = other DNA
gagaataccagcatcgtcttgatgcccttggaagtctcgcgcgtcctggattcgcggaaacgcgtacatccagctgacgagtcccaaataggacgaaacgcgc

SEQ ID NO: 46 - DNA, 103 bp, synthetic construct
  source 1..103: mol_type = other DNA
gcgcgtcctggattcgcggaaacgcgtacatccagctgacgagtccggaataccagcatcgtcttgatgcccttggaagtccgccaaataggacgaaacgcgc

SEQ ID NO: 47 - DNA, 101 bp, synthetic construct
  source 1..101: mol_type = other DNA
gcgcgtcctggattcgcggaaacgcgtacatccagctgacgagtcccaggaataccagcatcgtcttgatgcccttggaagtccaataggacgaaacgcgc

SEQ ID NO: 48 - DNA, 105 bp, synthetic construct
  source 1..105: mol_type = other DNA
gcgcgtcctggattcgcggaaacgcgtacatccagctgacgcgagaataccagcatcgtcttgatgcccttggaagtctcgagtcccaaataggacgaaacgcgc

SEQ ID NO: 49 - DNA, 101 bp, synthetic construct
  source 1..101: mol_type = other DNA
gcgcgtcctggattcgcggaaacgcgtacatagaataccagcatcgtcttgatgcccttggaagtctccagctgacgagtcccaaataggacgaaacgcgc

SEQ ID NO: 50 - DNA, 105 bp, synthetic construct
  source 1..105: mol_type = other DNA
gcgcgtcctggattcgcggaaacgcgtacatccagctgacgagaataccagcatcgtcttgatgcccttggaagtctcgcgagtcccaaataggacgaaacgcgc

SEQ ID NO: 51 - DNA, 105 bp, synthetic construct
  source 1..105: mol_type = other DNA
gcgcgtcctggattcgcggaaacgcgtacatccagctgacgagtcccaaataggacgaaaccgagaataccagcatcgtcttgatgcccttggaagtctcggcgc

SEQ ID NO: 52 - DNA, 103 bp, synthetic construct
  source 1..103: mol_type = other DNA
gcgcgtcctggattcgcggaaacgcgtacatccagctgacgagagtgataccagcatcgtcttgatgcccttggcagcacttcccaaataggacgaaacgcgc

SEQ ID NO: 53 - DNA, 103 bp, synthetic construct
  source 1..103: mol_type = other DNA
gcgcgtcctggattcgcggaaacgcgtacatccagagtgataccagcatcgtcttgatgcccttggcagcactctgacgagtcccaaataggacgaaacgcgc

SEQ ID NO: 54 - DNA, 103 bp, synthetic construct
  source 1..103: mol_type = other DNA
gagtgataccagcatcgtcttgatgcccttggcagcactcgcgtcctggattcgcggaaacgcgtacatccagctgacgagtcccaaataggacgaaacgcgc

SEQ ID NO: 55 - DNA, 105 bp, synthetic construct
  source 1..105: mol_type = other DNA
gcgcgtcctggattcgcggcgagaataccagcatcgtcttgatgcccttggaagtctcgaaacgcgtacatccagctgacgagtcccaaataggacgaaacgcgc

SEQ ID NO: 56 - DNA, 103 bp, synthetic construct
  source 1..103: mol_type = other DNA
gcgcgtcctggattcgcggaaacgcgtacatccagctgacgagtcccgagaataccagcatcgtcttgatgcccttggaagtctcaaataggacgaaacgcgc

SEQ ID NO: 57 - DNA, 101 bp, synthetic construct
  source 1..101: mol_type = other DNA
gcgcgtcctggattcgcggaaacgcgtacatccagctgacgagtcccaaataggacagaataccagcatcgtcttgatgcccttggaagtctgaaacgcgc

SEQ ID NO: 58 - DNA, 103 bp, synthetic construct
  source 1..103: mol_type = other DNA
gcgcgtcctggattcgcggaaacgcgtacatccagagaataccagcatcgtcttgatgcccttggaagtctcgctgacgagtcccaaataggacgaaacgcgc

SEQ ID NO: 59 - DNA, 105 bp, synthetic construct
  source 1..105: mol_type = other DNA
gcgcgtcctggattcccggaataccagcatcgtcttgatgcccttggaagtccgggcggaaacgcgtacatccagctgacgagtcccaaataggacgaaacgcgc

SEQ ID NO: 60 - DNA, 101 bp, synthetic construct
  source 1..101: mol_type = other DNA
gcgcgtcctggattcgcggaaacgcgtacatccagctgacgagtcccaaataggacgaaacgcgggaataccagcatcgtcttgatgcccttggaagtccc

SEQ ID NO: 61 - DNA, 101 bp, synthetic construct
  source 1..101: mol_type = other DNA
gcgcgtcctggattcgcggaaacgcgtacatccagctgacgagtcccaaataggacgaaacgcgcggaataccagcatcgtcttgatgcccttggaagtcc

SEQ ID NO: 62 - DNA, 105 bp, synthetic construct
  source 1..105: mol_type = other DNA
gcgcgtcctggattcgcggaaacgcccggaataccagcatcgtcttgatgcccttggaagtccgggtacatccagctgacgagtcccaaataggacgaaacgcgc

SEQ ID NO: 63 - DNA, 103 bp, synthetic construct
  source 1..103: mol_type = other DNA
gcgcgtcctggattcagtgataccagcatcgtcttgatgcccttggcagcactgcggaaacgcgtacatccagctgacgagtcccaaataggacgaaacgcgc

SEQ ID NO: 64 - DNA, 101 bp, synthetic construct
  source 1..101: mol_type = other DNA
gcgcgtcctggattcgcggaaacgcgtacagaataccagcatcgtcttgatgcccttggaagtctatccagctgacgagtcccaaataggacgaaacgcgc

SEQ ID NO: 65 - DNA, 76 bp, synthetic construct
  source 1..76: mol_type = other DNA
gctgtcaccggaataccagcatcgtcttgatgcccttggaagtccggtctgatgagtcccagaaggacgaaacagc

SEQ ID NO: 66 - DNA, 101 bp, synthetic construct
  source 1..101: mol_type = other DNA
gcgcgtcctggattcgcggaaagaataccagcatcgtcttgatgcccttggaagtctacgcgtacatccagctgacgagtcccaaataggacgaaacgcgc

SEQ ID NO: 67 - DNA, 103 bp, synthetic construct
  source 1..103: mol_type = other DNA
gcgcgtcctggattcgcggaaacgcgtacatccagtgataccagcatcgtcttgatgcccttggcagcactagctgacgagtcccaaataggacgaaacgcgc

SEQ ID NO: 68 - DNA, 105 bp, synthetic construct
  source 1..105: mol_type = other DNA
gcgcgtcctggattcgcccggaataccagcatcgtcttgatgcccttggaagtccggggaaacgcgtacatccagctgacgagtcccaaataggacgaaacgcgc

SEQ ID NO: 69 - DNA, 101 bp, synthetic construct
  source 1..101: mol_type = other DNA
gcgcgtcctggattcgcggaaacgcgtacatccagctgacgagtcccggaataccagcatcgtcttgatgcccttggaagtccaaataggacgaaacgcgc

SEQ ID NO: 70 - DNA, 103 bp, synthetic construct
  source 1..103: mol_type = other DNA
gcgcgtcctggattcgcggaaacgcgtacatccagctgagtgataccagcatcgtcttgatgcccttggcagcactacgagtcccaaataggacgaaacgcgc

SEQ ID NO: 71 - DNA, 103 bp, synthetic construct
  source 1..103: mol_type = other DNA
gcgcgtcctggattcgcggaaacgcgtacatccagctgacgagtcccaaataggacgaaacgcggaataccagcatcgtcttgatgcccttggaagtccgcgc

SEQ ID NO: 72 - DNA, 105 bp, synthetic construct
  source 1..105: mol_type = other DNA
gcgcgtcctggattcgcggaaacgcgtaccgagaataccagcatcgtcttgatgcccttggaagtctcgatccagctgacgagtcccaaataggacgaaacgcgc

SEQ ID NO: 73 - DNA, 103 bp, synthetic construct
  source 1..103: mol_type = other DNA
gcgcgtcctggattcgcggaaacgcgtacacggaataccagcatcgtcttgatgcccttggaagtccgtccagctgacgagtcccaaataggacgaaacgcgc

SEQ ID NO: 74 - DNA, 101 bp, synthetic construct
  source 1..101: mol_type = other DNA
gcgcgtcctggattcgcggaaacgcgtacatccagctgacgagtcagaataccagcatcgtcttgatgcccttggaagtctccaaataggacgaaacgcgc

SEQ ID NO: 75 - DNA, 103 bp, synthetic construct
  source 1..103: mol_type = other DNA
gcgcgtcctggattcgcggaaacgcgtacatccagctgacgagtcccaaataggacgaaagtgataccagcatcgtcttgatgcccttggcagcactacgcgc

SEQ ID NO: 76 - DNA, 103 bp, synthetic construct
  source 1..103: mol_type = other DNA
gcgcgtcctggattcgcgggagaataccagcatcgtcttgatgcccttggaagtctcaaacgcgtacatccagctgacgagtcccaaataggacgaaacgcgc

SEQ ID NO: 77 - DNA, 103 bp, synthetic construct
  source 1..103: mol_type = other DNA
gcgcgtccagtgataccagcatcgtcttgatgcccttggcagcacttggattcgcggaaacgcgtacatccagctgacgagtcccaaataggacgaaacgcgc

SEQ ID NO: 78 - DNA, 105 bp, synthetic construct
  source 1..105: mol_type = other DNA
gcgcgtcctggattcgcggaaacgcgtacatccagctgacgagtcccaaataggacgaaacgccgagaataccagcatcgtcttgatgcccttggaagtctcggc

SEQ ID NO: 79 - DNA, 105 bp, synthetic construct
  source 1..105: mol_type = other DNA
gcgcgtcctggattcgcggaaacgcgtacatccagcgagaataccagcatcgtcttgatgcccttggaagtctcgctgacgagtcccaaataggacgaaacgcgc

SEQ ID NO: 80 - DNA, 103 bp, synthetic construct
  source 1..103: mol_type = other DNA
gcgcgtcctggattcggagaataccagcatcgtcttgatgcccttggaagtctccggaaacgcgtacatccagctgacgagtcccaaataggacgaaacgcgc

SEQ ID NO: 81 - DNA, 101 bp, synthetic construct
  source 1..101: mol_type = other DNA
gcgcgtcctggattcgcggaaacgcgtacatccagctgacgagtcccaaggaataccagcatcgtcttgatgcccttggaagtccataggacgaaacgcgc

SEQ ID NO: 82 - DNA, 101 bp, synthetic construct
  source 1..101: mol_type = other DNA
gcgcgtcctggattcgagaataccagcatcgtcttgatgcccttggaagtctcggaaacgcgtacatccagctgacgagtcccaaataggacgaaacgcgc

SEQ ID NO: 83 - DNA, 101 bp, synthetic construct
  source 1..101: mol_type = other DNA
gcgcgtccggaataccagcatcgtcttgatgcccttggaagtcctggattcgcggaaacgcgtacatccagctgacgagtcccaaataggacgaaacgcgc

SEQ ID NO: 84 - DNA, 103 bp, synthetic construct
  source 1..103: mol_type = other DNA
gcgcgtcctggagagaataccagcatcgtcttgatgcccttggaagtctcttcgcggaaacgcgtacatccagctgacgagtcccaaataggacgaaacgcgc

SEQ ID NO: 85 - DNA, 101 bp, synthetic construct
  source 1..101: mol_type = other DNA
gcgcgtcctggattcgcggaaacgcgtacatccagaataccagcatcgtcttgatgcccttggaagtctagctgacgagtcccaaataggacgaaacgcgc

SEQ ID NO: 86 - DNA, 103 bp, synthetic construct
  source 1..103: mol_type = other DNA
gcgcgtcctggattcgcggaaacgcggaataccagcatcgtcttgatgcccttggaagtccgcgtacatccagctgacgagtcccaaataggacgaaacgcgc

SEQ ID NO: 87 - DNA, 105 bp, synthetic construct
  source 1..105: mol_type = other DNA
gcgcgtcctggattcgcggaaacgcgtacatccagctgacgagcgagaataccagcatcgtcttgatgcccttggaagtctcgtcccaaataggacgaaacgcgc

SEQ ID NO: 88 - DNA, 105 bp, synthetic construct
  source 1..105: mol_type = other DNA
gcgcgtcctggattcgcggaaacgcgtacatcccgagaataccagcatcgtcttgatgcccttggaagtctcgagctgacgagtcccaaataggacgaaacgcgc

SEQ ID NO: 89 - DNA, 103 bp, synthetic construct
  source 1..103: mol_type = other DNA
gcgcgtcctggattcgcggaaacgcgtacatccagctgacgagtcccaaataggaccggaataccagcatcgtcttgatgcccttggaagtccggaaacgcgc

SEQ ID NO: 90 - DNA, 103 bp, synthetic construct
  source 1..103: mol_type = other DNA
gcgcgtcctggaagtgataccagcatcgtcttgatgcccttggcagcactttcgcggaaacgcgtacatccagctgacgagtcccaaataggacgaaacgcgc

SEQ ID NO: 91 - DNA, 101 bp, synthetic construct
  source 1..101: mol_type = other DNA
ggaataccagcatcgtcttgatgcccttggaagtccgcgcgtcctggattcgcggaaacgcgtacatccagctgacgagtcccaaataggacgaaacgcgc

SEQ ID NO: 92 - DNA, 105 bp, synthetic construct
  source 1..105: mol_type = other DNA
gcgcgtcctggattcgcggaaacgcgtacatccagctgacgagtcccaaataggaccggaataccagcatcgtcttgatgcccttggaagtccggcgaaacgcgc

SEQ ID NO: 93 - DNA, 103 bp, synthetic construct
  source 1..103: mol_type = other DNA
agtgataccagcatcgtcttgatgcccttggcagcactgcgcgtcctggattcgcggaaacgcgtacatccagctgacgagtcccaaataggacgaaacgcgc

SEQ ID NO: 94 - DNA, 101 bp, synthetic construct
  source 1..101: mol_type = other DNA
gcgcgtcctggattcgcggaggaataccagcatcgtcttgatgcccttggaagtccaacgcgtacatccagctgacgagtcccaaataggacgaaacgcgc

SEQ ID NO: 95 - DNA, 101 bp, synthetic construct
  source 1..101: mol_type = other DNA
gcgcgtcagaataccagcatcgtcttgatgcccttggaagtctctggattcgcggaaacgcgtacatccagctgacgagtcccaaataggacgaaacgcgc

SEQ ID NO: 96 - DNA, 103 bp, synthetic construct
  source 1..103: mol_type = other DNA
gcgcgtcagtgataccagcatcgtcttgatgcccttggcagcactctggattcgcggaaacgcgtacatccagctgacgagtcccaaataggacgaaacgcgc

SEQ ID NO: 97 - DNA, 101 bp, synthetic construct
  source 1..101: mol_type = other DNA
gcgcgtcctggattcgcggaaacgcgtacatccagctgacgagtcccagaataccagcatcgtcttgatgcccttggaagtctaaataggacgaaacgcgc

SEQ ID NO: 98 - DNA, 105 bp, synthetic construct
  source 1..105: mol_type = other DNA
gcgcgtcctggattcgcggaaacgcgtacatccccggaataccagcatcgtcttgatgcccttggaagtccggagctgacgagtcccaaataggacgaaacgcgc

SEQ ID NO: 99 - DNA, 103 bp, synthetic construct
  source 1..103: mol_type = other DNA
gcgcgtcctggattcgcggaaacgcgtacatagtgataccagcatcgtcttgatgcccttggcagcactccagctgacgagtcccaaataggacgaaacgcgc

SEQ ID NO: 100 - DNA, 103 bp, synthetic construct
  source 1..103: mol_type = other DNA
gcgcgtcctggattcgcggaaacgcgtacatccagctgaccggaataccagcatcgtcttgatgcccttggaagtccggagtcccaaataggacgaaacgcgc

SEQ ID NO: 101 - DNA, 103 bp, synthetic construct
  source 1..103: mol_type = other DNA
gcgcgtcctggattcgcggaaacgcgtacatccagctgacgagtcccaaataggacgaacggaataccagcatcgtcttgatgcccttggaagtccgacgcgc

SEQ ID NO: 102 - DNA, 101 bp, synthetic construct
  source 1..101: mol_type = other DNA
gcgcgtcggaataccagcatcgtcttgatgcccttggaagtccctggattcgcggaaacgcgtacatccagctgacgagtcccaaataggacgaaacgcgc

SEQ ID NO: 103 - DNA, 101 bp, synthetic construct
  source 1..101: mol_type = other DNA
gcgcgtcctggattcgcggaaacgcgtacatccagctgacggaataccagcatcgtcttgatgcccttggaagtccgagtcccaaataggacgaaacgcgc

SEQ ID NO: 104 - DNA, 105 bp, synthetic construct
  source 1..105: mol_type = other DNA
gcgcgtcctggattcgcggaaacgcgtacatccagctgacgagtcccaaatcgagaataccagcatcgtcttgatgcccttggaagtctcgaggacgaaacgcgc

SEQ ID NO: 105 - DNA, 103 bp, synthetic construct
  source 1..103: mol_type = other DNA
gcgcgtcctggagaataccagcatcgtcttgatgcccttggaagtctcgattcgcggaaacgcgtacatccagctgacgagtcccaaataggacgaaacgcgc

SEQ ID NO: 106 - DNA, 105 bp, synthetic construct
  source 1..105: mol_type = other DNA
gcgcgtcctggattcgcggaaacgcgtacatccagctgaccggaataccagcatcgtcttgatgcccttggaagtccggcgagtcccaaataggacgaaacgcgc

SEQ ID NO: 107 - DNA, 103 bp, synthetic construct
  source 1..103: mol_type = other DNA
gcgcgtcctggattcgcggaaacgcgtacatcccggaataccagcatcgtcttgatgcccttggaagtccgagctgacgagtcccaaataggacgaaacgcgc

SEQ ID NO: 108 - DNA, 105 bp, synthetic construct
  source 1..105: mol_type = other DNA
gcgcgtcctggattcgcggccggaataccagcatcgtcttgatgcccttggaagtccggaaacgcgtacatccagctgacgagtcccaaataggacgaaacgcgc

SEQ ID NO: 109 - DNA, 105 bp, synthetic construct
  source 1..105: mol_type = other DNA
gcgccgagaataccagcatcgtcttgatgcccttggaagtctcggtcctggattcgcggaaacgcgtacatccagctgacgagtcccaaataggacgaaacgcgc

SEQ ID NO: 110 - DNA, 101 bp, synthetic construct
  source 1..101: mol_type = other DNA
gcgcgtcctggggaataccagcatcgtcttgatgcccttggaagtccattcgcggaaacgcgtacatccagctgacgagtcccaaataggacgaaacgcgc

SEQ ID NO: 111 - DNA, 103 bp, synthetic construct
  source 1..103: mol_type = other DNA
gcgcgtcctagtgataccagcatcgtcttgatgcccttggcagcactggattcgcggaaacgcgtacatccagctgacgagtcccaaataggacgaaacgcgc

SEQ ID NO: 112 - DNA, 101 bp, synthetic construct
  source 1..101: mol_type = other DNA
gcgcgtcctggattcgcggaaacgcgtacatccagggaataccagcatcgtcttgatgcccttggaagtccctgacgagtcccaaataggacaaaacgcgc

SEQ ID NO: 113 - DNA, 65 bp, synthetic construct
  source 1..65: mol_type = other DNA
gcgcgtcctggattcgcggaaacgcgtacatccagctgacgagtcccaaataggacgaaacgcgc

SEQ ID NO: 114 - DNA, 101 bp, synthetic construct
  source 1..101: mol_type = other DNA
gcgcgtcctggattcgcggaaacgcgtacatccagctgacgagtcccaaataggacgaggaataccagcatcgtcttgatgcccttggaagtccaacgcgc

SEQ ID NO: 115 - DNA, 101 bp, synthetic construct
  source 1..101: mol_type = other DNA
gcgcgtcctggattcgcggaaacgcgtacatccagctgacgagtcccaaataggacgagaataccagcatcgtcttgatgcccttggaagtctaaacgcgc

SEQ ID NO: 116 - DNA, 103 bp, synthetic construct
  source 1..103: mol_type = other DNA
gcgcgtcctggattcgcggaaacgcgtacatccagctgacgagtcccaaatagggagaataccagcatcgtcttgatgcccttggaagtctcacgaaacgcgc

SEQ ID NO: 117 - DNA, 103 bp, synthetic construct
  source 1..103: mol_type = other DNA
gcgcgtcctggatcggaataccagcatcgtcttgatgcccttggaagtccgtcgcggaaacgcgtacatccagctgacgagtcccaaataggacgaaacgcgc

SEQ ID NO: 118 - DNA, 103 bp, synthetic construct
  source 1..103: mol_type = other DNA
gcgcgtcctggattcgcggaaacgcgtacatccagctgacgagtcccaaataggacgagagaataccagcatcgtcttgatgcccttggaagtctcaacgcgc

SEQ ID NO: 119 - DNA, 101 bp, synthetic construct
  source 1..101: mol_type = other DNA
gcgcgtcctggattcgcggaaacgcgtacatccagagaataccagcatcgtcttgatgcccttggaagtctctgacgagtcccaaataggacgaaacgcgc

SEQ ID NO: 120 - DNA, 105 bp, synthetic construct
  source 1..105: mol_type = other DNA
gcgcgtccccggaataccagcatcgtcttgatgcccttggaagtccggtggattcgcggaaacgcgtacatccagctgacgagtcccaaataggacgaaacgcgc

SEQ ID NO: 121 - DNA, 103 bp, synthetic construct
  source 1..103: mol_type = other DNA
gcgcgtcctggattcgagaataccagcatcgtcttgatgcccttggaagtctcgcggaaacgcgtacatccagctgacgagtcccaaataggacgaaacgcgc

SEQ ID NO: 122 - DNA, 103 bp, synthetic construct
  source 1..103: mol_type = other DNA
gcgcgtcctggattcgcggaaacgcgtacatccagctgacgagtcccaaataggacgagaataccagcatcgtcttgatgcccttggaagtctcgaaacgcgc

SEQ ID NO: 123 - DNA, 103 bp, synthetic construct
  source 1..103: mol_type = other DNA
gcgcgtcctggattcgcggaaacgcgtcggaataccagcatcgtcttgatgcccttggaagtccgacatccagctgacgagtcccaaataggacgaaacgcgc

SEQ ID NO: 124 - DNA, 103 bp, synthetic construct
  source 1..103: mol_type = other DNA
gcgcgtcctggattcgcggaaacgcgtacatccagctgacgcggaataccagcatcgtcttgatgcccttggaagtccgagtcccaaataggacgaaacgcgc

SEQ ID NO: 125 - DNA, 105 bp, synthetic construct
  source 1..105: mol_type = other DNA
gcgcgtcctggattcgcggaaacgcgtacatccagctgacgagtcccggaataccagcatcgtcttgatgcccttggaagtccggccaaataggacgaaacgcgc

SEQ ID NO: 126 - DNA, 101 bp, synthetic construct
  source 1..101: mol_type = other DNA
gcgcgtcctggattcgcggaaacgcgtacaagaataccagcatcgtcttgatgcccttggaagtcttccagctgacgagtcccaaataggacgaaacgcgc

SEQ ID NO: 127 - DNA, 105 bp, synthetic construct
  source 1..105: mol_type = other DNA
gcgcgtcctggattcgcggaaacgcgtacatccagctgacgagtcccaaataggacgaaacgcgccggaataccagcatcgtcttgatgcccttggaagtccggc

SEQ ID NO: 128 - DNA, 103 bp, synthetic construct
  source 1..103: mol_type = other DNA
gcgcgtcctggattcgcggaaacgcgtaagtgataccagcatcgtcttgatgcccttggcagcactcatccagctgacgagtcccaaataggacgaaacgcgc

SEQ ID NO: 129 - DNA, 63 bp, synthetic construct
  source 1..63: mol_type = other DNA
gcgcgtcctggattccacttcgggtacatccagctgacgagtcccaaataggacgaaacgcgc

SEQ ID NO: 130 - DNA, 103 bp, synthetic construct
  source 1..103: mol_type = other DNA
gcgcgtcctggattcgcggaaacgcgtacatccagctgagaataccagcatcgtcttgatgcccttggaagtctcgacgagtcccaaataggacgaaacgcgc

SEQ ID NO: 131 - DNA, 101 bp, synthetic construct
  source 1..101: mol_type = other DNA
gcgcgtcctagaataccagcatcgtcttgatgcccttggaagtctggattcgcggaaacgcgtacatccagctgacgagtcccaaataggacgaaacgcgc

SEQ ID NO: 132 - DNA, 101 bp, synthetic construct
  source 1..101: mol_type = other DNA
gcgcgtcctggattcgcggaataccagcatcgtcttgatgcccttggaagtccggaaacgcgtacatccagctgacgagtcccaaataggacgaaacgcgc

SEQ ID NO: 133 - DNA, 103 bp, synthetic construct
  source 1..103: mol_type = other DNA
gcgcgagaataccagcatcgtcttgatgcccttggaagtctcgtcctggattcgcggaaacgcgtacatccagctgacgagtcccaaataggacgaaacgcgc

SEQ ID NO: 134 - DNA, 103 bp, synthetic construct
  source 1..103: mol_type = other DNA
gcgcgtcctggattcgcggaaacgcgtacatccagctgacgagtcggaataccagcatcgtcttgatgcccttggaagtccgcccaaataggacgaaacgcgc

SEQ ID NO: 135 - DNA, 103 bp, synthetic construct
  source 1..103: mol_type = other DNA
gcgcgtcctggattcgcggaaacgagtgataccagcatcgtcttgatgcccttggcagcactcgtacatccagctgacgagtcccaaataggacgaaacgcgc

SEQ ID NO: 136 - DNA, 103 bp, synthetic construct
  source 1..103: mol_type = other DNA
gcgcgtcctggattcgcggaaacgcgtacatccagctgacgagtcccaaataggacgaaacggagaataccagcatcgtcttgatgcccttggaagtctccgc

SEQ ID NO: 137 - DNA, 103 bp, synthetic construct
  source 1..103: mol_type = other DNA
gcgcgtcctggattcgcggaaacgcgtacgagaataccagcatcgtcttgatgcccttggaagtctcatccagctgacgagtcccaaataggacgaaacgcgc

SEQ ID NO: 138 - DNA, 103 bp, synthetic construct
  source 1..103: mol_type = other DNA
gcgcggaataccagcatcgtcttgatgcccttggaagtccgcgtcctggattcgcggaaacgcgtacatccagctgacgagtcccaaataggacgaaacgcgc

SEQ ID NO: 139 - DNA, 103 bp, synthetic construct
  source 1..103: mol_type = other DNA
ggagaataccagcatcgtcttgatgcccttggaagtctccgcgtcctggattcgcggaaacgcgtacatccagctgacgagtcccaaataggacgaaacgcgc

SEQ ID NO: 140 - DNA, 105 bp, synthetic construct
  source 1..105: mol_type = other DNA
gcgcgtcctggattcgcggaaacgcgtacatccagctgacgagtccggaataccagcatcgtcttgatgcccttggaagtccggcccaaataggacgaaacgcgc

SEQ ID NO: 141 - DNA, 105 bp, synthetic construct
  source 1..105: mol_type = other DNA
gcgcgtcctggattcgcggaaacgcgtacatccggaataccagcatcgtcttgatgcccttggaagtccggccagctgacgagtcccaaataggacgaaacgcgc

SEQ ID NO: 142 - DNA, 101 bp, synthetic construct
  source 1..101: mol_type = other DNA
gcgcgtcctggattcgcggaaacgcgtacggaataccagcatcgtcttgatgcccttggaagtccatccagctgacgagtcccaaataggacgaaacgcgc

SEQ ID NO: 143 - DNA, 103 bp, synthetic construct
  source 1..103: mol_type = other DNA
gcgcgtcctggattcgcggaaacgcgtacatccagctgacgagtgataccagcatcgtcttgatgcccttggcagcactagtcccaaataggacgaaacgcgc

SEQ ID NO: 144 - DNA, 105 bp, synthetic construct
  source 1..105: mol_type = other DNA
gcgcgcgagaataccagcatcgtcttgatgcccttggaagtctcgtcctggattcgcggaaacgcgtacatccagctgacgagtcccaaataggacgaaacgcgc

SEQ ID NO: 145 - DNA, 101 bp, synthetic construct
  source 1..101: mol_type = other DNA
gcgcggaataccagcatcgtcttgatgcccttggaagtccgtcctggattcgcggaaacgcgtacatccagctgacgagtcccaaataggacgaaacgcgc

SEQ ID NO: 146 - DNA, 101 bp, synthetic construct
  source 1..101: mol_type = other DNA
gcgcgtcctggattcgcggaaacgcgtacatccagctgacgagtcccaaataggaataccagcatcgtcttgatgcccttggaagtccggacgaaacgcgc

SEQ ID NO: 147 - DNA, 101 bp, synthetic construct
  source 1..101: mol_type = other DNA
gcgcgtcctggattcgcggaaacgcgtacatccagctgacgagtagaataccagcatcgtcttgatgcccttggaagtctcccaaataggacgaaacgcgc

SEQ ID NO: 148 - DNA, 101 bp, synthetic construct
  source 1..101: mol_type = other DNA
gcgcgtcctggattcgcggaaacgcgtacatccagctgacgagagaataccagcatcgtcttgatgcccttggaagtcttcccaaataggacgaaacgcgc

SEQ ID NO: 149 - DNA, 103 bp, synthetic construct
  source 1..103: mol_type = other DNA
gcgcggagaataccagcatcgtcttgatgcccttggaagtctctcctggattcgcggaaacgcgtacatccagctgacgagtcccaaataggacgaaacgcgc

SEQ ID NO: 150 - DNA, 101 bp, synthetic construct
  source 1..101: mol_type = other DNA
gcgcgtcctggattcgcggaaacgcgtacatccagctgacgagtcccaaataggacgaaaggaataccagcatcgtcttgatgcccttggaagtcccgcgc

SEQ ID NO: 151 - DNA, 105 bp, synthetic construct
  source 1..105: mol_type = other DNA
gcgcgtcctggattcgcggaaacgcgtaccggaataccagcatcgtcttgatgcccttggaagtccggcatccagctgacgagtcccaaataggacgaaacgcgc

SEQ ID NO: 152 - DNA, 101 bp, synthetic construct
  source 1..101: mol_type = other DNA
gcgcgtggaataccagcatcgtcttgatgcccttggaagtcccctggattcgcggaaacgcgtacatccagctgacgagtcccaaataggacgaaacgcgc

SEQ ID NO: 153 - DNA, 105 bp, synthetic construct
  source 1..105: mol_type = other DNA
cgagaataccagcatcgtcttgatgcccttggaagtctcggcgcgtcctggattcgcggaaacgcgtacatccagctgacgagtcccaaataggacgaaacgcgc

SEQ ID NO: 154 - DNA, 103 bp, synthetic construct
  source 1..103: mol_type = other DNA
gcgcgtcctggattcgcggaaacgcgagaataccagcatcgtcttgatgcccttggaagtctcgtacatccagctgacgagtcccaaataggacgaaacgcgc

SEQ ID NO: 155 - DNA, 105 bp, synthetic construct
  source 1..105: mol_type = other DNA
gcgcgtcctggattcgcggaaacgcgtacatccagctgacgagtcccaaataggacgagaataccagcatcgtcttgatgcccttggaagtctcgcgaaacgcgc

SEQ ID NO: 156 - DNA, 105 bp, synthetic construct
  source 1..105: mol_type = other DNA
gccggaataccagcatcgtcttgatgcccttggaagtccggcgcgtcctggattcgcggaaacgcgtacatccagctgacgagtcccaaataggacgaaacgcgc

SEQ ID NO: 157 - DNA, 101 bp, synthetic construct
  source 1..101: mol_type = other DNA
gcgcgtcctggattcgcggaaacgcgtaagaataccagcatcgtcttgatgcccttggaagtctcatccagctgacgagtcccaaataggacgaaacgcgc

SEQ ID NO: 158 - DNA, 103 bp, synthetic construct
  source 1..103: mol_type = other DNA
gcgcgtcctggattcgcggaaacgcgtacatccagctgacgagtcccaaataggacgagtgataccagcatcgtcttgatgcccttggcagcactaaacgcgc

SEQ ID NO: 159 - DNA, 103 bp, synthetic construct
  source 1..103: mol_type = other DNA
gcgcgtcctggattcgcggaataccagcatcgtcttgatgcccttggaagtccgcggaaacgcgtacatccagctgacgagtcccaaataggacgaaacgcgc

SEQ ID NO: 160 - DNA, 101 bp, synthetic construct
  source 1..101: mol_type = other DNA
gcgcgtcctggattcgcggaaacgcgtacatccagctgacgagggaataccagcatcgtcttgatgcccttggaagtcctcccaaataggacgaaacgcgc

SEQ ID NO: 161 - DNA, 105 bp, synthetic construct
  source 1..105: mol_type = other DNA
gcgcgtcctggattcgcggaaacgcgtacatccagctgacgagtcccaaataggacccggaataccagcatcgtcttgatgcccttggaagtccgggaaacgcgc

SEQ ID NO: 162 - DNA, 101 bp, synthetic construct
  source 1..101: mol_type = other DNA
gcgcgtcctggattcgcggaaacgcgtacatccagctgacgagtcccaaataggggaataccagcatcgtcttgatgcccttggaagtccacgaaacgcgc

SEQ ID NO: 163 - DNA, 105 bp, synthetic construct
  source 1..105: mol_type = other DNA
gcgcgtcctggattcgcggaaacgcgtacatccagccggaataccagcatcgtcttgatgcccttggaagtccggctgacgagtcccaaataggacgaaacgcgc

SEQ ID NO: 164 - DNA, 105 bp, synthetic construct
  source 1..105: mol_type = other DNA
gcgcgtcctggattcgcggaaacgcgtacatccagctgacgagtcccaaataggacgcgagaataccagcatcgtcttgatgcccttggaagtctcgaaacgcgc

SEQ ID NO: 165 - DNA, 103 bp, synthetic construct
  source 1..103: mol_type = other DNA
gcgcgtcctggattcgcggaaacgcgtagagaataccagcatcgtcttgatgcccttggaagtctccatccagctgacgagtcccaaataggacgaaacgcgc

SEQ ID NO: 166 - DNA, 105 bp, synthetic construct
  source 1..105: mol_type = other DNA
gcgcgtcctggattcgcggaaacgcgtacatccagctgacgagtcccacgagaataccagcatcgtcttgatgcccttggaagtctcgaataggacgaaacgcgc

SEQ ID NO: 167 - DNA, 101 bp, synthetic construct
  source 1..101: mol_type = other DNA
gcgcgtcctggattcgcggaaacgcgtacatccagggaataccagcatcgtcttgatgcccttggaagtccctgacgagtcccaaataggacgaaacgcgc

SEQ ID NO: 168 - DNA, 105 bp, synthetic construct
  source 1..105: mol_type = other DNA
gcgcgtcctggatcgagaataccagcatcgtcttgatgcccttggaagtctcgtcgcggaaacgcgtacatccagctgacgagtcccaaataggacgaaacgcgc

SEQ ID NO: 169 - DNA, 105 bp, synthetic construct
  source 1..105: mol_type = other DNA
gcgcgtcctggccggaataccagcatcgtcttgatgcccttggaagtccggattcgcggaaacgcgtacatccagctgacgagtcccaaataggacgaaacgcgc

SEQ ID NO: 170 - DNA, 101 bp, synthetic construct
  source 1..101: mol_type = other DNA
gcgcgtcctggattcgcggaaacgcgtacatccagctgacgagtcccaaataggacgaaacgcagaataccagcatcgtcttgatgcccttggaagtctgc

SEQ ID NO: 171 - DNA, 105 bp, synthetic construct
  source 1..105: mol_type = other DNA
gcgcgtcctggattcgcggaaacgcgtacacgagaataccagcatcgtcttgatgcccttggaagtctcgtccagctgacgagtcccaaataggacgaaacgcgc

SEQ ID NO: 172 - DNA, 101 bp, synthetic construct
  source 1..101: mol_type = other DNA
agaataccagcatcgtcttgatgcccttggaagtctgcgcgtcctggattcgcggaaacgcgtacatccagctgacgagtcccaaataggacgaaacgcgc

SEQ ID NO: 173 - DNA, 103 bp, synthetic construct
  source 1..103: mol_type = other DNA
gcgcgtcctggattcgcggaaacgcgtacatccagctgacgagtcccaaatagcggaataccagcatcgtcttgatgcccttggaagtccggacgaaacgcgc

SEQ ID NO: 174 - DNA, 103 bp, synthetic construct
  source 1..103: mol_type = other DNA
gcgcgtcctggattcgcggaaacgcgtacatccagccggaataccagcatcgtcttgatgcccttggaagtccgtgacgagtcccaaataggacgaaacgcgc

SEQ ID NO: 175 - DNA, 105 bp, synthetic construct
  source 1..105: mol_type = other DNA
gcgcgtcctggattcgcggaaacgcgtacatccagctgacgagtcccaaataggacgaaacgcgccgagaataccagcatcgtcttgatgcccttggaagtctcg

SEQ ID NO: 176 - DNA, 101 bp, synthetic construct
  source 1..101: mol_type = other DNA
gcgcgtcctggaataccagcatcgtcttgatgcccttggaagtccggattcgcggaaacgcgtacatccagctgacgagtcccaaataggacgaaacgcgc

SEQ ID NO: 177 - DNA, 103 bp, synthetic construct
  source 1..103: mol_type = other DNA
gcgcgtcctggattcgcggaaacgcgtacatccagctgacgagtcccaaataggacgaaacgcagtgataccagcatcgtcttgatgcccttggcagcactgc

SEQ ID NO: 178 - DNA, 101 bp, synthetic construct
  source 1..101: mol_type = other DNA
gcgcgtcctggattcgcggaaacgcgtacatccggaataccagcatcgtcttgatgcccttggaagtccagctgacgagtcccaaataggacgaaacgcgc

SEQ ID NO: 179 - DNA, 103 bp, synthetic construct
  source 1..103: mol_type = other DNA
gcgcgtcctggattcgcggaaacgcgtacatccagctcggaataccagcatcgtcttgatgcccttggaagtccggacgagtcccaaataggacgaaacgcgc

SEQ ID NO: 180 - DNA, 103 bp, synthetic construct
  source 1..103: mol_type = other DNA
gcgcgtcctggattcgcggaaacgcgtacatccagctggagaataccagcatcgtcttgatgcccttggaagtctcacgagtcccaaataggacgaaacgcgc

SEQ ID NO: 181 - DNA, 105 bp, synthetic construct
  source 1..105: mol_type = other DNA
gcgcgccggaataccagcatcgtcttgatgcccttggaagtccggtcctggattcgcggaaacgcgtacatccagctgacgagtcccaaataggacgaaacgcgc

SEQ ID NO: 182 - DNA, 103 bp, synthetic construct
  source 1..103: mol_type = other DNA
gcgcgtcctggattcgcggaaacgcggagaataccagcatcgtcttgatgcccttggaagtctctacatccagctgacgagtcccaaataggacgaaacgcgc

SEQ ID NO: 183 - DNA, 101 bp, synthetic construct
  source 1..101: mol_type = other DNA
gcgcgtcctggattcgcggaaacgcgtacatccagctgagaataccagcatcgtcttgatgcccttggaagtctacgagtcccaaataggacgaaacgcgc

SEQ ID NO: 184 - DNA, 101 bp, synthetic construct
  source 1..101: mol_type = other DNA
gagaataccagcatcgtcttgatgcccttggaagtctcgcgtcctggattcgcggaaacgcgtacatccagctgacgagtcccaaataggacgaaacgcgc

SEQ ID NO: 185 - DNA, 103 bp, synthetic construct
  source 1..103: mol_type = other DNA
gcgcgtcctggattcgcggaaacgcgtacatccagctgacgagtcccaaatacggaataccagcatcgtcttgatgcccttggaagtccgggacgaaacgcgc

SEQ ID NO: 186 - DNA, 105 bp, synthetic construct
  source 1..105: mol_type = other DNA
gcgcgtcctggattcgcgcgagaataccagcatcgtcttgatgcccttggaagtctcggaaacgcgtacatccagctgacgagtcccaaataggacgaaacgcgc

SEQ ID NO: 187 - DNA, 103 bp, synthetic construct
  source 1..103: mol_type = other DNA
gcgcgtcctggattcgcggaaacgcgtacatccagctgacgagtcccaaataggacgaaacgcgcgagaataccagcatcgtcttgatgcccttggaagtctc

SEQ ID NO: 188 - DNA, 103 bp, synthetic construct
  source 1..103: mol_type = other DNA
gcgcgtcctggattcgcggaaacgcgtacatccagctgacgagtcccaaataggacgaagagaataccagcatcgtcttgatgcccttggaagtctcacgcgc

SEQ ID NO: 189 - DNA, 101 bp, synthetic construct
  source 1..101: mol_type = other DNA
gcgcgtcctggattcggaataccagcatcgtcttgatgcccttggaagtccgcggaaacgcgtacatccagctgacgagtcccaaataggacgaaacgcgc

SEQ ID NO: 190 - DNA, 103 bp, synthetic construct
  source 1..103: mol_type = other DNA
gcgcgtcctggattcgcgagtgataccagcatcgtcttgatgcccttggcagcactgaaacgcgtacatccagctgacgagtcccaaataggacgaaacgcgc

SEQ ID NO: 191 - DNA, 105 bp, synthetic construct
  source 1..105: mol_type = other DNA
gcgcgtcctggattcgcggaacgagaataccagcatcgtcttgatgcccttggaagtctcgacgcgtacatccagctgacgagtcccaaataggacgaaacgcgc

SEQ ID NO: 192 - DNA, 101 bp, synthetic construct
  source 1..101: mol_type = other DNA
gcgcgtcctggattcgcggaaacgcgtacatccagctgacgagtcccaaatggaataccagcatcgtcttgatgcccttggaagtccaggacgaaacgcgc

SEQ ID NO: 193 - DNA, 105 bp, synthetic construct
  source 1..105: mol_type = other DNA
ccggaataccagcatcgtcttgatgcccttggaagtccgggcgcgtcctggattcgcggaaacgcgtacatccagctgacgagtcccaaataggacgaaacgcgc

SEQ ID NO: 194 - DNA, 103 bp, synthetic construct
  source 1..103: mol_type = other DNA
gcgcgtcctggattcgcggaaacgcgtacatccagctgacgagaataccagcatcgtcttgatgcccttggaagtctcgagtcccaaataggacgaaacgcgc

SEQ ID NO: 195 - DNA, 105 bp, synthetic construct
  source 1..105: mol_type = other DNA
gcgcgtcgagaataccagcatcgtcttgatgcccttggaagtctcgcctggattcgcggaaacgcgtacatccagctgacgagtcccaaataggacgaaacgcgc

SEQ ID NO: 196 - DNA, 103 bp, synthetic construct
  source 1..103: mol_type = other DNA
gcgcgtcctggattcgcggaaacgcgtacatccagctgacgagtcccaaatgagaataccagcatcgtcttgatgcccttggaagtctcaggacgaaacgcgc

SEQ ID NO: 197 - DNA, 65 bp, synthetic construct
  source 1..65: mol_type = other DNA
gcgcgtcctggattcgcggaaacgcgtacatccagctgacgagtcccaaataggacaaaacgcgc

SEQ ID NO: 198 - DNA, 101 bp, synthetic construct
  source 1..101: mol_type = other DNA
gcgcgtcctgagaataccagcatcgtcttgatgcccttggaagtctgattcgcggaaacgcgtacatccagctgacgagtcccaaataggacgaaacgcgc

SEQ ID NO: 199 - DNA, 103 bp, synthetic construct
  source 1..103: mol_type = other DNA
gcgcgtcctggattcgcggaaacagtgataccagcatcgtcttgatgcccttggcagcactgcgtacatccagctgacgagtcccaaataggacgaaacgcgc

SEQ ID NO: 200 - DNA, 105 bp, synthetic construct
  source 1..105: mol_type = other DNA
gcgcgtcctggattcgcggaaacgcgtacatccgagaataccagcatcgtcttgatgcccttggaagtctcgcagctgacgagtcccaaataggacgaaacgcgc

SEQ ID NO: 201 - DNA, 105 bp, synthetic construct
  source 1..105: mol_type = other DNA
gcgcgtcctggattcgcggaaacgcgagaataccagcatcgtcttgatgcccttggaagtctcgcgtacatccagctgacgagtcccaaataggacgaaacgcgc

SEQ ID NO: 202 - DNA, 103 bp, synthetic construct
  source 1..103: mol_type = other DNA
gcgcgtcctggatagtgataccagcatcgtcttgatgcccttggcagcacttcgcggaaacgcgtacatccagctgacgagtcccaaataggacgaaacgcgc

SEQ ID NO: 203 - DNA, 101 bp, synthetic construct
  source 1..101: mol_type = other DNA
gcgcgtcctggattcgcggaaacgcgtacatccagctgacgagtcccaaataggaagaataccagcatcgtcttgatgcccttggaagtctcgaaacgcgc

SEQ ID NO: 204 - DNA, 103 bp, synthetic construct
  source 1..103: mol_type = other DNA
gcgcgtcccggaataccagcatcgtcttgatgcccttggaagtccgtggattcgcggaaacgcgtacatccagctgacgagtcccaaataggacgaaacgcgc

SEQ ID NO: 205 - DNA, 101 bp, synthetic construct
  source 1..101: mol_type = other DNA
gcgcgtcctggattcgcggaaacgcgtacatccagctgacgagtcccaaataggacgaaacgcggaataccagcatcgtcttgatgcccttggaagtccgc

SEQ ID NO: 206 - DNA, 105 bp, synthetic construct
  source 1..105: mol_type = other DNA
gcgcgtcctggattcgccggaataccagcatcgtcttgatgcccttggaagtccggcggaaacgcgtacatccagctgacgagtcccaaataggacgaaacgcgc

SEQ ID NO: 207 - DNA, 103 bp, synthetic construct
  source 1..103: mol_type = other DNA
gcgcgtcctggattcgcggaaacgcgtacatccagctgacgagtcccaagagaataccagcatcgtcttgatgcccttggaagtctcataggacgaaacgcgc

SEQ ID NO: 208 - DNA, 105 bp, synthetic construct
  source 1..105: mol_type = other DNA
gcgcgtcctggattcgcggaaacgcgtacatccagctgacgagtcccaaaccggaataccagcatcgtcttgatgcccttggaagtccggtaggacgaaacgcgc

SEQ ID NO: 209 - DNA, 103 bp, synthetic construct
  source 1..103: mol_type = other DNA
gcgcgtcctggattcgcggagagaataccagcatcgtcttgatgcccttggaagtctcaacgcgtacatccagctgacgagtcccaaataggacgaaacgcgc

SEQ ID NO: 210 - DNA, 76 bp, synthetic construct
  source 1..76: mol_type = other DNA
gctgtcaccggaataccagcatcgtcttgatgcccttggaagtccggtctgatgagtccaaaaaggacgaaacagc

SEQ ID NO: 211 - DNA, 101 bp, synthetic construct
  source 1..101: mol_type = other DNA
gcgcgtcctggattcgggaataccagcatcgtcttgatgcccttggaagtcccggaaacgcgtacatccagctgacgagtcccaaataggacgaaacgcgc

SEQ ID NO: 212 - DNA, 101 bp, synthetic construct
  source 1..101: mol_type = other DNA
gcgcgtcctggattcagaataccagcatcgtcttgatgcccttggaagtctgcggaaacgcgtacatccagctgacgagtcccaaataggacgaaacgcgc

SEQ ID NO: 213 - DNA, 105 bp, synthetic construct
  source 1..105: mol_type = other DNA
gcgcgtcctggattcgcggaaacgcgtacatccagctcgagaataccagcatcgtcttgatgcccttggaagtctcggacgagtcccaaataggacgaaacgcgc

SEQ ID NO: 214 - DNA, 103 bp, synthetic construct
  source 1..103: mol_type = other DNA
gcgcgtcctggattcgcgagaataccagcatcgtcttgatgcccttggaagtctcggaaacgcgtacatccagctgacgagtcccaaataggacgaaacgcgc

SEQ ID NO: 215 - DNA, 52 bp, synthetic construct
  source 1..52: mol_type = other DNA
gctgtcaccggatgtgctttccggtacgtgaggtccgtgaggacgaaacagc

SEQ ID NO: 216 - DNA, 103 bp, synthetic construct
  source 1..103: mol_type = other DNA
gcgcgtcctggattcgcggaaacgcgtacatccagctgacgagtcagtgataccagcatcgtcttgatgcccttggcagcactccaaataggacgaaacgcgc

SEQ ID NO: 217 - DNA, 105 bp, synthetic construct
  source 1..105: mol_type = other DNA
gcgcgtcctggattccgagaataccagcatcgtcttgatgcccttggaagtctcggcggaaacgcgtacatccagctgacgagtcccaaataggacgaaacgcgc

SEQ ID NO: 218 - DNA, 103 bp, synthetic construct
  source 1..103: mol_type = other DNA
gcgcgtcctggattcgcggaaacgcgtacatccaagtgataccagcatcgtcttgatgcccttggcagcactgctgacgagtcccaaataggacgaaacgcgc

SEQ ID NO: 219 - DNA, 101 bp, synthetic construct
  source 1..101: mol_type = other DNA
gcgcgtcctggattcgcggaaacgcgtacatccagcggaataccagcatcgtcttgatgcccttggaagtcctgacgagtcccaaataggacgaaacgcgc

SEQ ID NO: 220 - DNA, 105 bp, synthetic construct
  source 1..105: mol_type = other DNA
gccgagaataccagcatcgtcttgatgcccttggaagtctcggcgtcctggattcgcggaaacgcgtacatccagctgacgagtcccaaataggacgaaacgcgc

SEQ ID NO: 221 - DNA, 103 bp, synthetic construct
  source 1..103: mol_type = other DNA
gcgcgtcctggattcgcggaaacgcgtacatccagctgacggaataccagcatcgtcttgatgcccttggaagtccgcgagtcccaaataggacgaaacgcgc

SEQ ID NO: 222 - DNA, 101 bp, synthetic construct
  source 1..101: mol_type = other DNA
gcgcgtcctggattcgcggaaacgcgtacatccagctgacagaataccagcatcgtcttgatgcccttggaagtctgagtcccaaataggacgaaacgcgc

SEQ ID NO: 223 - DNA, 105 bp, synthetic construct
  source 1..105: mol_type = other DNA
gcgcgtcctgcgagaataccagcatcgtcttgatgcccttggaagtctcggattcgcggaaacgcgtacatccagctgacgagtcccaaataggacgaaacgcgc

SEQ ID NO: 224 - DNA, 101 bp, synthetic construct
  source 1..101: mol_type = other DNA
gcgcgtcctggattcgcggaaacgcgtacatccagctgacgagtcccaaatagagaataccagcatcgtcttgatgcccttggaagtctgacgaaacgcgc

SEQ ID NO: 225 - DNA, 101 bp, synthetic construct
  source 1..101: mol_type = other DNA
gcgcgtcctggattcgcggaaacgcgtacatccagctgacgagtcccaaataagaataccagcatcgtcttgatgcccttggaagtctggacgaaacgcgc

SEQ ID NO: 226 - DNA, 103 bp, synthetic construct
  source 1..103: mol_type = other DNA
gcgcgtcctggattcgcggaaacgcgtacatccagctgaagtgataccagcatcgtcttgatgcccttggcagcactcgagtcccaaataggacgaaacgcgc

SEQ ID NO: 227 - DNA, 105 bp, synthetic construct
  source 1..105: mol_type = other DNA
gcgcgtccgagaataccagcatcgtcttgatgcccttggaagtctcgctggattcgcggaaacgcgtacatccagctgacgagtcccaaataggacgaaacgcgc

SEQ ID NO: 228 - DNA, 101 bp, synthetic construct
  source 1..101: mol_type = other DNA
gcgcgtcctggattcgcggaaacgcgtacatccagctgggaataccagcatcgtcttgatgcccttggaagtccacgagtcccaaataggacgaaacgcgc

SEQ ID NO: 229 - DNA, 105 bp, synthetic construct
  source 1..105: mol_type = other DNA
gcgccggaataccagcatcgtcttgatgcccttggaagtccggcgtcctggattcgcggaaacgcgtacatccagctgacgagtcccaaataggacgaaacgcgc

SEQ ID NO: 230 - DNA, 103 bp, synthetic construct
  source 1..103: mol_type = other DNA
gcgcgtcctgagaataccagcatcgtcttgatgcccttggaagtctcggattcgcggaaacgcgtacatccagctgacgagtcccaaataggacgaaacgcgc

SEQ ID NO: 231 - DNA, 103 bp, synthetic construct
  source 1..103: mol_type = other DNA
gcgagaataccagcatcgtcttgatgcccttggaagtctcgcgtcctggattcgcggaaacgcgtacatccagctgacgagtcccaaataggacgaaacgcgc

SEQ ID NO: 232 - DNA, 105 bp, synthetic construct
  source 1..105: mol_type = other DNA
gcccggaataccagcatcgtcttgatgcccttggaagtccgggcgtcctggattcgcggaaacgcgtacatccagctgacgagtcccaaataggacgaaacgcgc

SEQ ID NO: 233 - DNA, 103 bp, synthetic construct
  source 1..103: mol_type = other DNA
gcagtgataccagcatcgtcttgatgcccttggcagcactgcgtcctggattcgcggaaacgcgtacatccagctgacgagtcccaaataggacgaaacgcgc

SEQ ID NO: 234 - DNA, 103 bp, synthetic construct
  source 1..103: mol_type = other DNA
gcgcgtcctggattcgcggaaacgcgtacatccagctgacgagtcccaaataggacgaaaccggaataccagcatcgtcttgatgcccttggaagtccggcgc

SEQ ID NO: 235 - DNA, 103 bp, synthetic construct
  source 1..103: mol_type = other DNA
gcgcgtcctggattcgcggaaacgcgtacatccagctgacgagtcccaaataggagagaataccagcatcgtcttgatgcccttggaagtctccgaaacgcgc

SEQ ID NO: 236 - DNA, 105 bp, synthetic construct
  source 1..105: mol_type = other DNA
gcgcgtcctggattcgcggaaacgcgtacaccggaataccagcatcgtcttgatgcccttggaagtccggtccagctgacgagtcccaaataggacgaaacgcgc

SEQ ID NO: 237 - DNA, 103 bp, synthetic construct
  source 1..103: mol_type = other DNA
gcgcgtcctggattcgcggaaacgcgtacatccagctgacggagaataccagcatcgtcttgatgcccttggaagtctcagtcccaaataggacgaaacgcgc

SEQ ID NO: 238 - DNA, 101 bp, synthetic construct
  source 1..101: mol_type = other DNA
gcgcgtcctggattagaataccagcatcgtcttgatgcccttggaagtctcgcggaaacgcgtacatccagctgacgagtcccaaataggacgaaacgcgc

SEQ ID NO: 239 - DNA, 105 bp, synthetic construct
  source 1..105: mol_type = other DNA
gcgcgtcctggattcgcggaaacgcgtacatccagctgacccggaataccagcatcgtcttgatgcccttggaagtccgggagtcccaaataggacgaaacgcgc

SEQ ID NO: 240 - DNA, 105 bp, synthetic construct
  source 1..105: mol_type = other DNA
gcgcgtcctggattcgcggaaacgcgtacatccagctgacgagtccccgagaataccagcatcgtcttgatgcccttggaagtctcgaaataggacgaaacgcgc

SEQ ID NO: 241 - DNA, 105 bp, synthetic construct
  source 1..105: mol_type = other DNA
gcgcgtcctccggaataccagcatcgtcttgatgcccttggaagtccggggattcgcggaaacgcgtacatccagctgacgagtcccaaataggacgaaacgcgc

SEQ ID NO: 242 - DNA, 105 bp, synthetic construct
  source 1..105: mol_type = other DNA
gcgcgtcctggattccggaataccagcatcgtcttgatgcccttggaagtccggcgcggaaacgcgtacatccagctgacgagtcccaaataggacgaaacgcgc

SEQ ID NO: 243 - DNA, 105 bp, synthetic construct
  source 1..105: mol_type = other DNA
gcgcgtcctggattcgcggaaacgcgtacatccagcccggaataccagcatcgtcttgatgcccttggaagtccggtgacgagtcccaaataggacgaaacgcgc

SEQ ID NO: 244 - DNA, 105 bp, synthetic construct
  source 1..105: mol_type = other DNA
gcgcgtcctggattcgcggaaacgcgtccggaataccagcatcgtcttgatgcccttggaagtccggacatccagctgacgagtcccaaataggacgaaacgcgc

SEQ ID NO: 245 - DNA, 101 bp, synthetic construct
  source 1..101: mol_type = other DNA
gggaataccagcatcgtcttgatgcccttggaagtcccgcgtcctggattcgcggaaacgcgtacatccagctgacgagtcccaaataggacgaaacgcgc

SEQ ID NO: 246 - DNA, 103 bp, synthetic construct
  source 1..103: mol_type = other DNA
gcgcgtcctggattcgcggagaataccagcatcgtcttgatgcccttggaagtctcgaaacgcgtacatccagctgacgagtcccaaataggacgaaacgcgc

SEQ ID NO: 247 - DNA, 101 bp, synthetic construct
  source 1..101: mol_type = other DNA
gcgcgtcctggattcgcggaaacgcgtacatccagctgaggaataccagcatcgtcttgatgcccttggaagtcccgagtcccaaataggacgaaacgcgc

SEQ ID NO: 248 - DNA, 103 bp, synthetic construct
  source 1..103: mol_type = other DNA
gcgcgtcctggattcgcggaaacgcgtacatccagctgacgagtccagtgataccagcatcgtcttgatgcccttggcagcactcaaataggacgaaacgcgc

SEQ ID NO: 249 - DNA, 105 bp, synthetic construct
  source 1..105: mol_type = other DNA
gcgcgtcctggattcgcggaaacgcgtacatccaccggaataccagcatcgtcttgatgcccttggaagtccgggctgacgagtcccaaataggacgaaacgcgc

SEQ ID NO: 250 - DNA, 103 bp, synthetic construct
  source 1..103: mol_type = other DNA
gcgcgagtgataccagcatcgtcttgatgcccttggcagcacttcctggattcgcggaaacgcgtacatccagctgacgagtcccaaataggacgaaacgcgc

SEQ ID NO: 251 - DNA, 105 bp, synthetic construct
  source 1..105: mol_type = other DNA
gcgcgtcctggaccggaataccagcatcgtcttgatgcccttggaagtccggttcgcggaaacgcgtacatccagctgacgagtcccaaataggacgaaacgcgc

SEQ ID NO: 252 - DNA, 103 bp, synthetic construct
  source 1..103: mol_type = other DNA
gcgcgtcctggattcgcggaagtgataccagcatcgtcttgatgcccttggcagcactaacgcgtacatccagctgacgagtcccaaataggacgaaacgcgc

SEQ ID NO: 253 - DNA, 101 bp, synthetic construct
  source 1..101: mol_type = other DNA
gcgcgtcctggattcgcggaaacgcgtacatggaataccagcatcgtcttgatgcccttggaagtccccagctgacgagtcccaaataggacgaaacgcgc

SEQ ID NO: 254 - DNA, 101 bp, synthetic construct
  source 1..101: mol_type = other DNA
gcgcgtcctggattcgcggaaacgcgtacatccagcagaataccagcatcgtcttgatgcccttggaagtcttgacgagtcccaaataggacgaaacgcgc

SEQ ID NO: 255 - DNA, 105 bp, synthetic construct
  source 1..105: mol_type = other DNA
gcgcgtcctggattcgcggaaacgcgtacatccagctgacgagtcccaaataggacgaaaccggaataccagcatcgtcttgatgcccttggaagtccggcgcgc

SEQ ID NO: 256 - DNA, 103 bp, synthetic construct
  source 1..103: mol_type = other DNA
gcgcgtcctggattcgcggaaacgcgtacatgagaataccagcatcgtcttgatgcccttggaagtctcccagctgacgagtcccaaataggacgaaacgcgc

SEQ ID NO: 257 - DNA, 103 bp, synthetic construct
  source 1..103: mol_type = other DNA
gcgcgtcctggattcgcggaaacgccggaataccagcatcgtcttgatgcccttggaagtccggtacatccagctgacgagtcccaaataggacgaaacgcgc

SEQ ID NO: 258 - DNA, 103 bp, synthetic construct
  source 1..103: mol_type = other DNA
gcgcgtcctggattagtgataccagcatcgtcttgatgcccttggcagcactcgcggaaacgcgtacatccagctgacgagtcccaaataggacgaaacgcgc

SEQ ID NO: 259 - DNA, 103 bp, synthetic construct
  source 1..103: mol_type = other DNA
gcgcgtcctggattcgcggaaacgcgtacatccagctgacgagtcccaaataggacgaaaagtgataccagcatcgtcttgatgcccttggcagcactcgcgc

SEQ ID NO: 260 - DNA, 105 bp, synthetic construct
  source 1..105: mol_type = other DNA
gcgcgtcctggattcgcggaaacgccggaataccagcatcgtcttgatgcccttggaagtccggcgtacatccagctgacgagtcccaaataggacgaaacgcgc

SEQ ID NO: 261 - DNA, 105 bp, synthetic construct
  source 1..105: mol_type = other DNA
gcgcgtcctggattcgcggaaacgcgtacatccagctgacgagtcccaacgagaataccagcatcgtcttgatgcccttggaagtctcgataggacgaaacgcgc

SEQ ID NO: 262 - DNA, 76 bp, synthetic construct
  source 1..76: mol_type = other DNA
gctgtcaccggaataccagcatcgtcttgatgcccttggaagtccggtctgatgagtcccataaggacgaaacagc

SEQ ID NO: 263 - DNA, 103 bp, synthetic construct
  source 1..103: mol_type = other DNA
gcgcgtcctggattcgcggaaacgcgtacatccagctgacgagtcccaaaagtgataccagcatcgtcttgatgcccttggcagcacttaggacgaaacgcgc

SEQ ID NO: 264 - DNA, 101 bp, synthetic construct
  source 1..101: mol_type = other DNA
gcgcgtcctggattcgcggaaacgcgtacatccagctagaataccagcatcgtcttgatgcccttggaagtctgacgagtcccaaataggacgaaacgcgc

SEQ ID NO: 265 - DNA, 101 bp, synthetic construct
  source 1..101: mol_type = other DNA
gcgcgtcctggattcgcggaaacgcgtacatccagctgacgagtcccaaaggaataccagcatcgtcttgatgcccttggaagtcctaggacgaaacgcgc

SEQ ID NO: 266 - DNA, 105 bp, synthetic construct
  source 1..105: mol_type = other DNA
gcgcgtcctggattcgcggaaacgcgcgagaataccagcatcgtcttgatgcccttggaagtctcgtacatccagctgacgagtcccaaataggacgaaacgcgc

SEQ ID NO: 267 - DNA, 101 bp, synthetic construct
  source 1..101: mol_type = other DNA
gcgcgtcctggattcgcggaaacgcgtacatccagctgacgagtggaataccagcatcgtcttgatgcccttggaagtcccccaaataggacgaaacgcgc

SEQ ID NO: 268 - DNA, 103 bp, synthetic construct
  source 1..103: mol_type = other DNA
gcgcgtcctggattcgcggaaacgcgtacatccagctgacgagtcccaaataggacgaaacgcggagaataccagcatcgtcttgatgcccttggaagtctcc

SEQ ID NO: 269 - DNA, 101 bp, synthetic construct
  source 1..101: mol_type = other DNA
gcgcgtcctggattcgcggaaacgcgagaataccagcatcgtcttgatgcccttggaagtcttacatccagctgacgagtcccaaataggacgaaacgcgc

SEQ ID NO: 270 - DNA, 101 bp, synthetic construct
  source 1..101: mol_type = other DNA
gcgcgtcctggattcgcggaaacgcgtacatccagctgacgagtcccaaataggacgaagaataccagcatcgtcttgatgcccttggaagtctaacgcgc

SEQ ID NO: 271 - DNA, 105 bp, synthetic construct
  source 1..105: mol_type = other DNA
gcgcgtcctggattcgcggaaacgcgtacatccagctgacgagtcccaaccggaataccagcatcgtcttgatgcccttggaagtccggataggacgaaacgcgc

SEQ ID NO: 272 - DNA, 101 bp, synthetic construct
  source 1..101: mol_type = other DNA
gcgcgtcctggattcgcggaaacgcgtacatccagctgacgagtcccaaataggacgaaacgcgcagaataccagcatcgtcttgatgcccttggaagtct

SEQ ID NO: 273 - DNA, 101 bp, synthetic construct
  source 1..101: mol_type = other DNA
gcgcgtcctggattcgcgggaataccagcatcgtcttgatgcccttggaagtccgaaacgcgtacatccagctgacgagtcccaaataggacgaaacgcgc

SEQ ID NO: 274 - DNA, 103 bp, synthetic construct
  source 1..103: mol_type = other DNA
gcgcgtcctggattcgcggaaacgcgtacatccagctgacgagtcccaaagagaataccagcatcgtcttgatgcccttggaagtctctaggacgaaacgcgc

SEQ ID NO: 275 - DNA, 103 bp, synthetic construct
  source 1..103: mol_type = other DNA
gcgcgtcctggattgagaataccagcatcgtcttgatgcccttggaagtctccgcggaaacgcgtacatccagctgacgagtcccaaataggacgaaacgcgc

SEQ ID NO: 276 - DNA, 101 bp, synthetic construct
  source 1..101: mol_type = other DNA
gcgcgtcctggattcgcggaaacgcgtacatccagctgacgaagaataccagcatcgtcttgatgcccttggaagtctgtcccaaataggacgaaacgcgc

SEQ ID NO: 277 - DNA, 103 bp, synthetic construct
  source 1..103: mol_type = other DNA
gcgcgtcctggattcgcggaaacgcgtacatccgagaataccagcatcgtcttgatgcccttggaagtctcagctgacgagtcccaaataggacgaaacgcgc

SEQ ID NO: 278 - DNA, 103 bp, synthetic construct
  source 1..103: mol_type = other DNA
gcgcgtcctggattcgagtgataccagcatcgtcttgatgcccttggcagcactcggaaacgcgtacatccagctgacgagtcccaaataggacgaaacgcgc

SEQ ID NO: 279 - DNA, 105 bp, synthetic construct
  source 1..105: mol_type = other DNA
gcgcgtcctggattcgcggaaacgcgtacatccagctgacgagtcccaaataggacgacgagaataccagcatcgtcttgatgcccttggaagtctcgaacgcgc

SEQ ID NO: 280 - DNA, 101 bp, synthetic construct
  source 1..101: mol_type = other DNA
gcgcgtcctggattcgcggaaacgcgtacatccagctgacgagtcccaaataggaggaataccagcatcgtcttgatgcccttggaagtcccgaaacgcgc

SEQ ID NO: 281 - DNA, 105 bp, synthetic construct
  source 1..105: mol_type = other DNA
gcgcgtcctggattcgcggaaacgcgtacatccagctgacgagtcccaaatacgagaataccagcatcgtcttgatgcccttggaagtctcgggacgaaacgcgc

SEQ ID NO: 282 - DNA, 105 bp, synthetic construct
  source 1..105: mol_type = other DNA
gcgcgtcctggattcgcgagaataccagcatcgtcttgatgcccttggaagtctcgcggaaacgcgtacatccagctgacgagtcccaaataggacgaaacgcgc

SEQ ID NO: 283 - DNA, 101 bp, synthetic construct
  source 1..101: mol_type = other DNA
gcgcgtcctggaagaataccagcatcgtcttgatgcccttggaagtctttcgcggaaacgcgtacatccagctgacgagtcccaaataggacgaaacgcgc

SEQ ID NO: 284 - DNA, 101 bp, synthetic construct
  source 1..101: mol_type = other DNA
gcgcgtcctggattcgcggaaacggaataccagcatcgtcttgatgcccttggaagtccgcgtacatccagctgacgagtcccaaataggacgaaacgcgc

SEQ ID NO: 285 - DNA, 103 bp, synthetic construct
  source 1..103: mol_type = other DNA
gcgcgtcctggattcgcggaaacgcagtgataccagcatcgtcttgatgcccttggcagcactgtacatccagctgacgagtcccaaataggacgaaacgcgc

SEQ ID NO: 286 - DNA, 101 bp, synthetic construct
  source 1..101: mol_type = other DNA
gcgcgtcctggattcgcggaaaagaataccagcatcgtcttgatgcccttggaagtctcgcgtacatccagctgacgagtcccaaataggacgaaacgcgc

SEQ ID NO: 287 - DNA, 103 bp, synthetic construct
  source 1..103: mol_type = other DNA
gcgcgtcctggattcgcggaaacgcgtacatccagctgacgaagtgataccagcatcgtcttgatgcccttggcagcactgtcccaaataggacgaaacgcgc

SEQ ID NO: 288 - DNA, 103 bp, synthetic construct
  source 1..103: mol_type = other DNA
gcgcgtcctggattcgcggaaacgcgtacatccagctgacgagtcccaaataagtgataccagcatcgtcttgatgcccttggcagcactggacgaaacgcgc

SEQ ID NO: 289 - DNA, 101 bp, synthetic construct
  source 1..101: mol_type = other DNA
gcgcgtcctggattcgcggaaacgcgtacatccaggaataccagcatcgtcttgatgcccttggaagtccgctgacgagtcccaaataggacgaaacgcgc

SEQ ID NO: 290 - DNA, 105 bp, synthetic construct
  source 1..105: mol_type = other DNA
gcgcgtcctggattcgcggaaacgcgtacatccagctgacgaccggaataccagcatcgtcttgatgcccttggaagtccgggtcccaaataggacgaaacgcgc

SEQ ID NO: 291 - DNA, 76 bp, synthetic construct; identical to SEQ ID NO: 262
  source 1..76: mol_type = other DNA

SEQ ID NO: 292 - DNA, 103 bp, synthetic construct
  source 1..103: mol_type = other DNA
gcgcgtcggaataccagcatcgtcttgatgcccttggaagtccgcctggattcgcggaaacgcgtacatccagctgacgagtcccaaataggacgaaacgcgc

SEQ ID NO: 293 - DNA, 101 bp, synthetic construct
  source 1..101: mol_type = other DNA
gcgggaataccagcatcgtcttgatgcccttggaagtcccgtcctggattcgcggaaacgcgtacatccagctgacgagtcccaaataggacgaaacgcgc

SEQ ID NO: 294 - DNA, 103 bp, synthetic construct
  source 1..103: mol_type = other DNA
gcgcgtcctggattcgcggaaacgagaataccagcatcgtcttgatgcccttggaagtctcgcgtacatccagctgacgagtcccaaataggacgaaacgcgc

SEQ ID NO: 295 - DNA, 101 bp, synthetic construct
  source 1..101: mol_type = other DNA
gcgcgtcctggattcgcggaaaggaataccagcatcgtcttgatgcccttggaagtcccgcgtacatccagctgacgagtcccaaataggacgaaacgcgc

SEQ ID NO: 296 - DNA, 101 bp, synthetic construct
  source 1..101: mol_type = other DNA
gcgcgtcctggattcgcggaaacgcgtacatccagctgacgagtcccaaataggagaataccagcatcgtcttgatgcccttggaagtctacgaaacgcgc

SEQ ID NO: 297 - DNA, 101 bp, synthetic construct
  source 1..101: mol_type = other DNA
gcgcgggaataccagcatcgtcttgatgcccttggaagtcctcctggattcgcggaaacgcgtacatccagctgacgagtcccaaataggacgaaacgcgc

SEQ ID NO: 298 - DNA, 103 bp, synthetic construct
  source 1..103: mol_type = other DNA
gccggaataccagcatcgtcttgatgcccttggaagtccggcgtcctggattcgcggaaacgcgtacatccagctgacgagtcccaaataggacgaaacgcgc

SEQ ID NO: 299 - DNA, 103 bp, synthetic construct
  source 1..103: mol_type = other DNA
gcgcgtagtgataccagcatcgtcttgatgcccttggcagcactcctggattcgcggaaacgcgtacatccagctgacgagtcccaaataggacgaaacgcgc

SEQ ID NO: 300 - DNA, 105 bp, synthetic construct
  source 1..105: mol_type = other DNA
gcgcgtcctggcgagaataccagcatcgtcttgatgcccttggaagtctcgattcgcggaaacgcgtacatccagctgacgagtcccaaataggacgaaacgcgc

SEQ ID NO: 301 - DNA, 103 bp, synthetic construct
  source 1..103: mol_type = other DNA
gcgagtgataccagcatcgtcttgatgcccttggcagcactcgtcctggattcgcggaaacgcgtacatccagctgacgagtcccaaataggacgaaacgcgc

SEQ ID NO: 302 - DNA, 101 bp, synthetic construct
  source 1..101: mol_type = other DNA
gcgcgtcctggattcgcggaaacgcgtacatccagctgacgagaataccagcatcgtcttgatgcccttggaagtctagtcccaaataggacgaaacgcgc

SEQ ID NO: 303 - DNA, 103 bp, synthetic construct
  source 1..103: mol_type = other DNA
gcgccggaataccagcatcgtcttgatgcccttggaagtccggtcctggattcgcggaaacgcgtacatccagctgacgagtcccaaataggacgaaacgcgc

SEQ ID NO: 304 - DNA, 105 bp, synthetic construct
  source 1..105: mol_type = other DNA
gcgcgagaataccagcatcgtcttgatgcccttggaagtctcgcgtcctggattcgcggaaacgcgtacatccagctgacgagtcccaaataggacgaaacgcgc

SEQ ID NO: 305 - DNA, 103 bp, synthetic construct
  source 1..103: mol_type = other DNA
gcgcgtcctggattcgcggaaacgcgtacatcagtgataccagcatcgtcttgatgcccttggcagcactcagctgacgagtcccaaataggacgaaacgcgc

SEQ ID NO: 306 - DNA, 101 bp, synthetic construct
  source 1..101: mol_type = other DNA
gcgcgggaataccagcatcgtcttgatgcccttggaagtcctcctggattcgcggaaacgcgtacatccagctgacgagtcccaaataggacaaaacgcgc

SEQ ID NO: 307 - DNA, 103 bp, synthetic construct
  source 1..103: mol_type = other DNA
gcgcgtcctggattcgcggaaacgcgtacatccagctgacgagtgagaataccagcatcgtcttgatgcccttggaagtctccccaaataggacgaaacgcgc

SEQ ID NO: 308 - DNA, 103 bp, synthetic construct
  source 1..103: mol_type = other DNA
gcgcgtcctggattcgcggaaacgcgtacatccagcagtgataccagcatcgtcttgatgcccttggcagcacttgacgagtcccaaataggacgaaacgcgc

SEQ ID NO: 309 - DNA, 101 bp, synthetic construct
  source 1..101: mol_type = other DNA
gcgcgagaataccagcatcgtcttgatgcccttggaagtcttcctggattcgcggaaacgcgtacatccagctgacgagtcccaaataggacgaaacgcgc

SEQ ID NO: 310 - DNA, 101 bp, synthetic construct
  source 1..101: mol_type = other DNA
gcgcgtcctggattcgcggaaacgcgtacatccagctgacgagtcccaaatagaataccagcatcgtcttgatgcccttggaagtctaggacgaaacgcgc

SEQ ID NO: 311 - DNA, 105 bp, synthetic construct
  source 1..105: mol_type = other DNA
gcgcgtcctggattcgcggaaacgcgtcgagaataccagcatcgtcttgatgcccttggaagtctcgacatccagctgacgagtcccaaataggacgaaacgcgc

SEQ ID NO: 312 - DNA, 101 bp, synthetic construct
  source 1..101: mol_type = other DNA
gcgcgtcctggattcgcggaaacgcgtacatccagctgacgaggaataccagcatcgtcttgatgcccttggaagtccgtcccaaataggacgaaacgcgc

SEQ ID NO: 313 - DNA, 103 bp, synthetic construct
  source 1..103: mol_type = other DNA
gcgcgtcctggattcgcggaaacgcgtacatccagctgacgagtcccaacggaataccagcatcgtcttgatgcccttggaagtccgataggacgaaacgcgc

SEQ ID NO: 314 - DNA, 101 bp, synthetic construct
  source 1..101: mol_type = other DNA
gcgcgtcctggattcgcggaaacgcgtacatccagctgacgagtcccaaatagggaataccagcatcgtcttgatgcccttggaagtccgacgaaacgcgc

SEQ ID NO: 315 - DNA, 105 bp, synthetic construct
  source 1..105: mol_type = other DNA
gcgcgtcctggattcgcggaaacgcgtacatccagctgacgagtcccaaataggacgaacgagaataccagcatcgtcttgatgcccttggaagtctcgacgcgc

SEQ ID NO: 316 - DNA, 101 bp, synthetic construct
  source 1..101: mol_type = other DNA
gcgcgtcctggatggaataccagcatcgtcttgatgcccttggaagtcctcgcggaaacgcgtacatccagctgacgagtcccaaataggacaaaacgcgc

SEQ ID NO: 317 - DNA, 105 bp, synthetic construct
  source 1..105: mol_type = other DNA
gcgcgtcctggattcgcggaaacgcgtacatccagctgacgagtcccaaatagccggaataccagcatcgtcttgatgcccttggaagtccgggacgaaacgcgc

SEQ ID NO: 318 - DNA, 103 bp, synthetic construct
  source 1..103: mol_type = other DNA
gcggaataccagcatcgtcttgatgcccttggaagtccgcgcgtcctggattcgcggaaacgcgtacatccagctgacgagtcccaaataggacgaaacgcgc

SEQ ID NO: 319 - DNA, 103 bp, synthetic construct
  source 1..103: mol_type = other DNA
gcgcgtcctggattcgcggaaacgcgtacagagaataccagcatcgtcttgatgcccttggaagtctctccagctgacgagtcccaaataggacgaaacgcgc

SEQ ID NO: 320 - DNA, 103 bp, synthetic construct
  source 1..103: mol_type = other DNA
gcgcgtcctggattcgcggaaacgcgtacatccagctgacgagtagtgataccagcatcgtcttgatgcccttggcagcactcccaaataggacgaaacgcgc

SEQ ID NO: 321 - DNA, 103 bp, synthetic construct
  source 1..103: mol_type = other DNA
gcgcgtcctggattcgcggaaacgcgtacatccagctagtgataccagcatcgtcttgatgcccttggcagcactgacgagtcccaaataggacgaaacgcgc

SEQ ID NO: 322 - DNA, 101 bp, synthetic construct
  source 1..101: mol_type = other DNA
gcgcgtcctggattcgcggagaataccagcatcgtcttgatgcccttggaagtctaaacgcgtacatccagctgacgagtcccaaataggacgaaacgcgc

SEQ ID NO: 323 - DNA, 103 bp, synthetic construct
  source 1..103: mol_type = other DNA
gcgcgtcctggattcgcgcggaataccagcatcgtcttgatgcccttggaagtccggaaacgcgtacatccagctgacgagtcccaaataggacgaaacgcgc

SEQ ID NO: 324 - DNA, 103 bp, synthetic construct
  source 1..103: mol_type = other DNA
gcgcgtcctggattcgcggaaaccggaataccagcatcgtcttgatgcccttggaagtccggcgtacatccagctgacgagtcccaaataggacgaaacgcgc

SEQ ID NO: 325 - DNA, 101 bp, synthetic construct
  source 1..101: mol_type = other DNA
gcgcgtcctggattcgcggaaacgcgtacatccagctgacgagtcccaaataggaggaataccagcatcgtcttgatgcccttggaagtcccaaaacgcgc

SEQ ID NO: 326 - DNA, 76 bp, synthetic construct
  source 1..76: mol_type = other DNA
gctgtcaccggaataccagcatcgtcttgatgcccttggaagtccggtctgatgagtcccttgaggacgaaacagc

SEQ ID NO: 327 - DNA, 103 bp, synthetic construct
  source 1..103: mol_type = other DNA
gcgcgtcctggattcgcggaaacgcgtagtgataccagcatcgtcttgatgcccttggcagcactacatccagctgacgagtcccaaataggacgaaacgcgc

SEQ ID NO: 328 - DNA, 103 bp, synthetic construct
  source 1..103: mol_type = other DNA
gcgcgtcctggattcgcggaaacgcgtacatccagctgacagtgataccagcatcgtcttgatgcccttggcagcactgagtcccaaataggacgaaacgcgc

SEQ ID NO: 329 - DNA, 103 bp, synthetic construct
  source 1..103: mol_type = other DNA
gcgcgtcctggattcgcggaaacgcgtacatccagctgacgagtcccaaataggacgaaacagtgataccagcatcgtcttgatgcccttggcagcactgcgc

SEQ ID NO: 330 - DNA, 103 bp, synthetic construct
  source 1..103: mol_type = other DNA
gcgcgtcctgggagaataccagcatcgtcttgatgcccttggaagtctcattcgcggaaacgcgtacatccagctgacgagtcccaaataggacgaaacgcgc

SEQ ID NO: 331 - DNA, 101 bp, synthetic construct
  source 1..101: mol_type = other DNA
gcgcgtcctgggaataccagcatcgtcttgatgcccttggaagtccgattcgcggaaacgcgtacatccagctgacgagtcccaaataggacgaaacgcgc

SEQ ID NO: 332 - DNA, 101 bp, synthetic construct
  source 1..101: mol_type = other DNA
gcgcgtcctggattcgcggaaacgcgtacatccagctgacgagtcggaataccagcatcgtcttgatgcccttggaagtccccaaataggacgaaacgcgc

SEQ ID NO: 333 - DNA, 101 bp, synthetic construct
  source 1..101: mol_type = other DNA
gcgcgtcctggattcgcggaaacgcgtacatccagctgacgagtcccaaagaataccagcatcgtcttgatgcccttggaagtctataggacgaaacgcgc

SEQ ID NO: 334 - DNA, 105 bp, synthetic construct
  source 1..105: mol_type = other DNA
gcgcgtcctggattcgcggaaacgcgtacccggaataccagcatcgtcttgatgcccttggaagtccggatccagctgacgagtcccaaataggacgaaacgcgc

SEQ ID NO: 335 - DNA, 101 bp, synthetic construct
  source 1..101: mol_type = other DNA
gcgcgtcctggattcgcggaaacgcgtaggaataccagcatcgtcttgatgcccttggaagtcccatccagctgacgagtcccaaataggacgaaacgcgc

SEQ ID NO: 336 - DNA, 103 bp, synthetic construct
  source 1..103: mol_type = other DNA
gcgcgtcctggattcgcggaaagtgataccagcatcgtcttgatgcccttggcagcactacgcgtacatccagctgacgagtcccaaataggacgaaacgcgc

SEQ ID NO: 337 - DNA, 101 bp, synthetic construct
  source 1..101: mol_type = other DNA
gcgcgtagaataccagcatcgtcttgatgcccttggaagtctcctggattcgcggaaacgcgtacatccagctgacgagtcccaaataggacgaaacgcgc

SEQ ID NO: 338 - DNA, 105 bp, synthetic construct
  source 1..105: mol_type = other DNA
gcgcgtcctggattcgcggaaacgagaataccagcatcgtcttgatgcccttggaagtctcgcgcgtacatccagctgacgagtcccaaataggacgaaacgcgc

SEQ ID NO: 339 - DNA, 101 bp, synthetic construct
  source 1..101: mol_type = other DNA
gcgcgtcctggattggaataccagcatcgtcttgatgcccttggaagtcccgcggaaacgcgtacatccagctgacgagtcccaaataggacgaaacgcgc

SEQ ID NO: 340 - DNA, 103 bp, synthetic construct
  source 1..103: mol_type = other DNA
gcgcagtgataccagcatcgtcttgatgcccttggcagcactgtcctggattcgcggaaacgcgtacatccagctgacgagtcccaaataggacgaaacgcgc

SEQ ID NO: 341 - DNA, 101 bp, synthetic construct
  source 1..101: mol_type = other DNA
gcggaataccagcatcgtcttgatgcccttggaagtccgcgtcctggattcgcggaaacgcgtacatccagctgacgagtcccaaataggacgaaacgcgc

SEQ ID NO: 342 - DNA, 103 bp, synthetic construct
  source 1..103: mol_type = other DNA
gcgcgtcctggattcgcggaaacgcgtacggaataccagcatcgtcttgatgcccttggaagtccgcatccagctgacgagtcccaaataggacgaaacgcgc

SEQ ID NO: 343 - DNA, 103 bp, synthetic construct
  source 1..103: mol_type = other DNA
gcgcgtccggaataccagcatcgtcttgatgcccttggaagtccgctggattcgcggaaacgcgtacatccagctgacgagtcccaaataggacgaaacgcgc

SEQ ID NO: 344 - DNA, 105 bp, synthetic construct
  source 1..105: mol_type = other DNA
gcgcgtcctggattcgcggaaacccggaataccagcatcgtcttgatgcccttggaagtccgggcgtacatccagctgacgagtcccaaataggacgaaacgcgc

SEQ ID NO: 345 - DNA, 101 bp, synthetic construct
  source 1..101: mol_type = other DNA
gcgcgtcctggattcgcggaaacgcgtacatccagctgacgagtcccaaataggacgaaacgcgagaataccagcatcgtcttgatgcccttggaagtctc

SEQ ID NO: 346 - DNA, 101 bp, synthetic construct
  source 1..101: mol_type = other DNA
gcgcgtcctggattcgcggaaacgcgtacatccagctggaataccagcatcgtcttgatgcccttggaagtccgacgagtcccaaataggacgaaacgcgc

SEQ ID NO: 347 - DNA, 101 bp, synthetic construct
  source 1..101: mol_type = other DNA
gcgcgtcctggattcgcggaaacgcagaataccagcatcgtcttgatgcccttggaagtctgtacatccagctgacgagtcccaaataggacgaaacgcgc

SEQ ID NO: 348 - DNA, 105 bp, synthetic construct
  source 1..105: mol_type = other DNA
gcgcgtcctggattcgcggaaacgcgtacatccagctgacgacgagaataccagcatcgtcttgatgcccttggaagtctcggtcccaaataggacgaaacgcgc

SEQ ID NO: 349 - DNA, 103 bp, synthetic construct
  source 1..103: mol_type = other DNA
gcgcgtcctggattcgcggaaacgcgtacatccagctgacgaggagaataccagcatcgtcttgatgcccttggaagtctctcccaaataggacgaaacgcgc

SEQ ID NO: 350 - DNA, 101 bp, synthetic construct
  source 1..101: mol_type = other DNA
gcgcagaataccagcatcgtcttgatgcccttggaagtctgtcctggattcgcggaaacgcgtacatccagctgacgagtcccaaataggacgaaacgcgc

SEQ ID NO: 351 - DNA, 103 bp, synthetic construct
  source 1..103: mol_type = other DNA
gcgcgtcctggattcgcggaaacgcgtacatccagcgagaataccagcatcgtcttgatgcccttggaagtctctgacgagtcccaaataggacgaaacgcgc

SEQ ID NO: 352 - DNA, 105 bp, synthetic construct
  source 1..105: mol_type = other DNA
gcgcgtcctggattcgcggaaacgcgtacatccagctgacgagtcccaaataggacgaaacgcgcgagaataccagcatcgtcttgatgcccttggaagtctcgc

SEQ ID NO: 353 - DNA, 105 bp, synthetic construct
  source 1..105: mol_type = other DNA
gcgcgtcctggattcgcggaaacgcgtacatccagctgacgagtcccaaataggacgaaacgccggaataccagcatcgtcttgatgcccttggaagtccggcgc

SEQ ID NO: 354 - DNA, 103 bp, synthetic construct
  source 1..103: mol_type = other DNA
gcgcgtcctggattcgcggaaacgcgtacatccagctgacgagtcccaaataggagtgataccagcatcgtcttgatgcccttggcagcactacgaaacgcgc

SEQ ID NO: 355 - DNA, 103 bp, synthetic construct
  source 1..103: mol_type = other DNA
gcgcgtcctggattcgcggaaacgcgtacatcgagaataccagcatcgtcttgatgcccttggaagtctccagctgacgagtcccaaataggacgaaacgcgc

SEQ ID NO: 356 - DNA, 103 bp, synthetic construct
  source 1..103: mol_type = other DNA
gcgcgtcctggattcgcggaaacgcgtacatccagctgacgagtcccaaataggacgaaagagaataccagcatcgtcttgatgcccttggaagtctccgcgc

SEQ ID NO: 357 - DNA, 101 bp, synthetic construct
  source 1..101: mol_type = other DNA
gcgcgtcctggattcgcggaaacgcgtacatccagctgacgagtcccaaataggacgggaataccagcatcgtcttgatgcccttggaagtccaaacgcgc

SEQ ID NO: 358 - DNA, 103 bp, synthetic construct
  source 1..103: mol_type = other DNA
gcgcgtcctggattcgcggaaacgcgtacatccagctgacgagcggaataccagcatcgtcttgatgcccttggaagtccgtcccaaataggacgaaacgcgc

SEQ ID NO: 359 - DNA, 101 bp, synthetic construct
  source 1..101: mol_type = other DNA
gcgcgtcctggatagaataccagcatcgtcttgatgcccttggaagtcttcgcggaaacgcgtacatccagctgacgagtcccaaataggacgaaacgcgc

SEQ ID NO: 360 - DNA, 101 bp, synthetic construct
  source 1..101: mol_type = other DNA
gcagaataccagcatcgtcttgatgcccttggaagtctgcgtcctggattcgcggaaacgcgtacatccagctgacgagtcccaaataggacgaaacgcgc

SEQ ID NO: 361 - DNA, 105 bp, synthetic construct
  source 1..105: mol_type = other DNA
gcgcgtcctggattcgcggaaacgcgtacatccagctgacgagtcccaaataggacgaaacgcccggaataccagcatcgtcttgatgcccttggaagtccgggc

SEQ ID NO: 362 - DNA, 101 bp, synthetic construct
  source 1..101: mol_type = other DNA
gcgcgtcctggaggaataccagcatcgtcttgatgcccttggaagtccttcgcggaaacgcgtacatccagctgacgagtcccaaataggacgaaacgcgc

SEQ ID NO: 363 - DNA, 103 bp, synthetic construct
  source 1..103: mol_type = other DNA
gcgcgtcctggattcgcggaaacgcgtacatccagctgacgagtcccacggaataccagcatcgtcttgatgcccttggaagtccgaataggacgaaacgcgc

SEQ ID NO: 364 - DNA, 101 bp, synthetic construct
  source 1..101: mol_type = other DNA
gcgcgtcctggattcgcggaaacgcgtggaataccagcatcgtcttgatgcccttggaagtccacatccagctgacgagtcccaaataggacgaaacgcgc

SEQ ID NO: 365 - DNA, 101 bp, synthetic construct
  source 1..101: mol_type = other DNA
gcgagaataccagcatcgtcttgatgcccttggaagtctcgtcctggattcgcggaaacgcgtacatccagctgacgagtcccaaataggacgaaacgcgc

SEQ ID NO: 366 - DNA, 103 bp, synthetic construct
  source 1..103: mol_type = other DNA
gcgcgtcctggattcgcggaagagaataccagcatcgtcttgatgcccttggaagtctcacgcgtacatccagctgacgagtcccaaataggacgaaacgcgc

SEQ ID NO: 367 - DNA, 101 bp, synthetic construct
  source 1..101: mol_type = other DNA
gcgcgtcctggatggaataccagcatcgtcttgatgcccttggaagtcctcgcggaaacgcgtacatccagctgacgagtcccaaataggacgaaacgcgc

SEQ ID NO: 368 - DNA, 105 bp, synthetic construct
  source 1..105: mol_type = other DNA
gcgcgtcctggattcgcggaaacgcgtacatccagctgacgagtcccgagaataccagcatcgtcttgatgcccttggaagtctcgcaaataggacgaaacgcgc

SEQ ID NO: 369 - DNA, 103 bp, synthetic construct
  source 1..103: mol_type = other DNA
gcgcgtcctggattcggaataccagcatcgtcttgatgcccttggaagtccgcgcggaaacgcgtacatccagctgacgagtcccaaataggacgaaacgcgc

SEQ ID NO: 370 - DNA, 101 bp, synthetic construct
  source 1..101: mol_type = other DNA
gcgcgtcctggattcgcggaaacgcgtacatccagctgacgagtcccaaataggacgaaacgggaataccagcatcgtcttgatgcccttggaagtcccgc

SEQ ID NO: 371 - DNA, 101 bp, synthetic construct
  source 1..101: mol_type = other DNA
gcgcgtcctggattcgcggaaacgcgtacatccagctgacgagtcccaaaagaataccagcatcgtcttgatgcccttggaagtcttaggacgaaacgcgc

SEQ ID NO: 372 - DNA, 105 bp, synthetic construct
  source 1..105: mol_type = other DNA
gcgcgtcctggattcgcggaaacgcgtacatccagctgacgagtcccaaacgagaataccagcatcgtcttgatgcccttggaagtctcgtaggacgaaacgcgc

SEQ ID NO: 373 - DNA, 103 bp, synthetic construct
  source 1..103: mol_type = other DNA
gcgcgtcctggattcgcggaaacgcgtacatccagctgacgagtcccaagtgataccagcatcgtcttgatgcccttggcagcactaataggacgaaacgcgc

SEQ ID NO: 374 - DNA, 105 bp, synthetic construct
  source 1..105: mol_type = other DNA
gcgcgtcctggattcgcggaaacgcgtacatccagctgccggaataccagcatcgtcttgatgcccttggaagtccggacgagtcccaaataggacgaaacgcgc

SEQ ID NO: 375 - DNA, 105 bp, synthetic construct
  source 1..105: mol_type = other DNA
gcgcgtcctggattcgcggaaacgcgtacatccagctccggaataccagcatcgtcttgatgcccttggaagtccgggacgagtcccaaataggacgaaacgcgc

SEQ ID NO: 376 - DNA, 101 bp, synthetic construct
  source 1..101: mol_type = other DNA
gcgcgtcctggattcgcggaaacgcgtacatccagctgaagaataccagcatcgtcttgatgcccttggaagtctcgagtcccaaataggacgaaacgcgc

SEQ ID NO: 377 - DNA, 103 bp, synthetic construct
  source 1..103: mol_type = other DNA
gcgcgtcctggattcgcggaaacgcgtacatccagctgacgagtcccaaataggacggagaataccagcatcgtcttgatgcccttggaagtctcaaacgcgc

SEQ ID NO: 378 - DNA, 105 bp, synthetic construct
  source 1..105: mol_type = other DNA
gcgcgtcctggattcgcggaaacgcgtacatcccggaataccagcatcgtcttgatgcccttggaagtccggcagctgacgagtcccaaataggacgaaacgcgc

SEQ ID NO: 379 - DNA, 101 bp, synthetic construct
  source 1..101: mol_type = other DNA
gcgcgtcctggattcgcggaaacgcggaataccagcatcgtcttgatgcccttggaagtccgtacatccagctgacgagtcccaaataggacgaaacgcgc

SEQ ID NO: 380 - DNA, 105 bp, synthetic construct
  source 1..105: mol_type = other DNA
gcgcgtcctggattcgcggaaacgccgagaataccagcatcgtcttgatgcccttggaagtctcggtacatccagctgacgagtcccaaataggacgaaacgcgc

SEQ ID NO: 381 - DNA, 105 bp, synthetic construct
  source 1..105: mol_type = other DNA
gcgcgtcctggattcgcggaaacgcgtacatccagctgacgagtcccaaataggacgaaacgcgcccggaataccagcatcgtcttgatgcccttggaagtccgg

SEQ ID NO: 382 - DNA, 105 bp, synthetic construct
  source 1..105: mol_type = other DNA
gcgcgtcctggattcgcggaaacgcgtacatccagctgacgccggaataccagcatcgtcttgatgcccttggaagtccggagtcccaaataggacgaaacgcgc

SEQ ID NO: 383 - DNA, 103 bp, synthetic construct
  source 1..103: mol_type = other DNA
cggaataccagcatcgtcttgatgcccttggaagtccggcgcgtcctggattcgcggaaacgcgtacatccagctgacgagtcccaaataggacgaaacgcgc

SEQ ID NO: 384 - DNA, 103 bp, synthetic construct
  source 1..103: mol_type = other DNA
gcgcgtcctggattcgcggaaacgcgtacatccagctgacgagtcgagaataccagcatcgtcttgatgcccttggaagtctcccaaataggacgaaacgcgc

SEQ ID NO: 385 - DNA, 105 bp, synthetic construct
  source 1..105: mol_type = other DNA
gcgcgtcctggatccggaataccagcatcgtcttgatgcccttggaagtccggtcgcggaaacgcgtacatccagctgacgagtcccaaataggacgaaacgcgc

SEQ ID NO: 386 - DNA, 101 bp, synthetic construct
  source 1..101: mol_type = other DNA
gcgcgtcctggattcgcggaaacgcgtacatccagctgacgggaataccagcatcgtcttgatgcccttggaagtccagtcccaaataggacgaaacgcgc

SEQ ID NO: 387 - DNA, 105 bp, synthetic construct
  source 1..105: mol_type = other DNA
gcgcgtcctggattcgcggaaacgcgccggaataccagcatcgtcttgatgcccttggaagtccggtacatccagctgacgagtcccaaataggacgaaacgcgc

SEQ ID NO: 388 - DNA, 103 bp, synthetic construct
  source 1..103: mol_type = other DNA
gcgcgtcctgagtgataccagcatcgtcttgatgcccttggcagcactgattcgcggaaacgcgtacatccagctgacgagtcccaaataggacgaaacgcgc

SEQ ID NO: 389 - DNA, 103 bp, synthetic construct
  source 1..103: mol_type = other DNA
gcgcgtcctggattcgcggaaacgcgtaccggaataccagcatcgtcttgatgcccttggaagtccgatccagctgacgagtcccaaataggacgaaacgcgc

SEQ ID NO: 390 - DNA, 105 bp, synthetic construct
  source 1..105: mol_type = other DNA
gcgcgtccggaataccagcatcgtcttgatgcccttggaagtccggcctggattcgcggaaacgcgtacatccagctgacgagtcccaaataggacgaaacgcgc

SEQ ID NO: 391 - DNA, 103 bp, synthetic construct
  source 1..103: mol_type = other DNA
gcgcgtcctggattcgcggaaacgcgtacatccagctgacgagtcccagagaataccagcatcgtcttgatgcccttggaagtctcaataggacgaaacgcgc

SEQ ID NO: 392 - DNA, 103 bp, synthetic construct
  source 1..103: mol_type = other DNA
gcgcgtcctggattcgcggacggaataccagcatcgtcttgatgcccttggaagtccgaacgcgtacatccagctgacgagtcccaaataggacgaaacgcgc

SEQ ID NO: 393 - DNA, 103 bp, synthetic construct
  source 1..103: mol_type = other DNA
gcgcgtcctggattcgcggaaacgcgtacatccagctgacgagtcccaaataggcggaataccagcatcgtcttgatgcccttggaagtccgacgaaacgcgc

SEQ ID NO: 394 - DNA, 101 bp, synthetic construct
  source 1..101: mol_type = other DNA
gcgcgtcctggattcgcagaataccagcatcgtcttgatgcccttggaagtctggaaacgcgtacatccagctgacgagtcccaaataggacgaaacgcgc

SEQ ID NO: 395 - DNA, 103 bp, synthetic construct
  source 1..103: mol_type = other DNA
gcgcgtcctggattcgcggaaacgcgtacatccagctgacgagtcccaaataggagaataccagcatcgtcttgatgcccttggaagtctcgacgaaacgcgc

SEQ ID NO: 396 - DNA, 103 bp, synthetic construct
  source 1..103: mol_type = other DNA
gcgcgtcctggattcgcggaaacgcgtacatccagctgcggaataccagcatcgtcttgatgcccttggaagtccgacgagtcccaaataggacgaaacgcgc

SEQ ID NO: 397 - DNA, 103 bp, synthetic construct
  source 1..103: mol_type = other DNA
gcgcgtcctggattcgcggaaacgcgtacatccagctgacgagtcccaaataggacgaaacgcgccggaataccagcatcgtcttgatgcccttggaagtccg

SEQ ID NO: 398 - DNA, 105 bp, synthetic construct
  source 1..105: mol_type = other DNA
gcgcgtcctggattcgcggacgagaataccagcatcgtcttgatgcccttggaagtctcgaacgcgtacatccagctgacgagtcccaaataggacgaaacgcgc

SEQ ID NO: 399 - DNA, 105 bp, synthetic construct
  source 1..105: mol_type = other DNA
gcgcgtcctggattcgcggaaacgcgtacatccacgagaataccagcatcgtcttgatgcccttggaagtctcggctgacgagtcccaaataggacgaaacgcgc

SEQ ID NO: 400 - DNA, 105 bp, synthetic construct
  source 1..105: mol_type = other DNA
gcgcgtcctggattcgcggaaccggaataccagcatcgtcttgatgcccttggaagtccggacgcgtacatccagctgacgagtcccaaataggacgaaacgcgc

SEQ ID NO: 401 - DNA, 103 bp, synthetic construct
  source 1..103: mol_type = other DNA
gcgcgtcctggattcgcggaaacgcgcggaataccagcatcgtcttgatgcccttggaagtccgtacatccagctgacgagtcccaaataggacgaaacgcgc

SEQ ID NO: 402 - DNA, 105 bp, synthetic construct
  source 1..105: mol_type = other DNA
gcgcgtcctggattcgagaataccagcatcgtcttgatgcccttggaagtctcgcgcggaaacgcgtacatccagctgacgagtcccaaataggacgaaacgcgc

SEQ ID NO: 403 - DNA, 103 bp, synthetic construct
  source 1..103: mol_type = other DNA
gcgcgtcctcggaataccagcatcgtcttgatgcccttggaagtccgggattcgcggaaacgcgtacatccagctgacgagtcccaaataggacgaaacgcgc

SEQ ID NO: 404 - DNA, 101 bp, synthetic construct
  source 1..101: mol_type = other DNA
gcgcgtcctggattcgcggaaggaataccagcatcgtcttgatgcccttggaagtccacgcgtacatccagctgacgagtcccaaataggacgaaacgcgc

SEQ ID NO: 405 - DNA, 105 bp, synthetic construct
  source 1..105: mol_type = other DNA
gcgcgtcctggattcgcggaaacgcgtacgagaataccagcatcgtcttgatgcccttggaagtctcgcatccagctgacgagtcccaaataggacgaaacgcgc

SEQ ID NO: 406 - DNA, 103 bp, synthetic construct
  source 1..103: mol_type = other DNA
gcgcgtcctggattcgcggaaacgcgtacatccagctgacgagtccccggaataccagcatcgtcttgatgcccttggaagtccgaaataggacgaaacgcgc

SEQ ID NO: 407 - DNA, 101 bp, synthetic construct
  source 1..101: mol_type = other DNA
gcgcgtcctggattcgcggaaacgcgtacatccagctgacgagtcccaaataggacggaataccagcatcgtcttgatgcccttggaagtccgaaacgcgc

SEQ ID NO: 408 - DNA, 101 bp, synthetic construct
  source 1..101: mol_type = other DNA
gcgcgtcctggattcgcggaaacgcgggaataccagcatcgtcttgatgcccttggaagtcctacatccagctgacgagtcccaaataggacgaaacgcgc

SEQ ID NO: 409 - DNA, 105 bp, synthetic construct
  source 1..105: mol_type = other DNA
gcgcgtcctggattcgccgagaataccagcatcgtcttgatgcccttggaagtctcgggaaacgcgtacatccagctgacgagtcccaaataggacgaaacgcgc

SEQ ID NO: 410 - DNA, 103 bp, synthetic construct
  source 1..103: mol_type = other DNA
gcgcgtcctggattcgcggaaacgcgtacatccagctgacgagtcccagtgataccagcatcgtcttgatgcccttggcagcactaaataggacgaaacgcgc

SEQ ID NO: 411 - DNA, 105 bp, synthetic construct
  source 1..105: mol_type = other DNA
gcgcgtcctggattcgcggaaacgcgtacatccagctgacgagtcccaaatagcgagaataccagcatcgtcttgatgcccttggaagtctcggacgaaacgcgc

SEQ ID NO: 412 - DNA, 103 bp, synthetic construct
  source 1..103: mol_type = other DNA
gcgcgtcctggattcgcggagtgataccagcatcgtcttgatgcccttggcagcactaaacgcgtacatccagctgacgagtcccaaataggacgaaacgcgc

SEQ ID NO: 413 - DNA, 101 bp, synthetic construct
  source 1..101: mol_type = other DNA
gcgcgtcctggattcgcggaaacgcgtagaataccagcatcgtcttgatgcccttggaagtctacatccagctgacgagtcccaaataggacgaaacgcgc

SEQ ID NO: 414 - DNA, 105 bp, synthetic construct
  source 1..105: mol_type = other DNA
gcgcgtcctggattcgcggaaacgcgtacatccagctgacgagtcccccggaataccagcatcgtcttgatgcccttggaagtccggaaataggacgaaacgcgc

SEQ ID NO: 415 - DNA, 105 bp, synthetic construct
  source 1..105: mol_type = other DNA
gcgcgtcctggattcgcggaccggaataccagcatcgtcttgatgcccttggaagtccggaacgcgtacatccagctgacgagtcccaaataggacgaaacgcgc

SEQ ID NO: 416 - DNA, 103 bp, synthetic construct
  source 1..103: mol_type = other DNA
gcgcgtcctggacggaataccagcatcgtcttgatgcccttggaagtccgttcgcggaaacgcgtacatccagctgacgagtcccaaataggacgaaacgcgc

SEQ ID NO: 417 - DNA, 103 bp, synthetic construct
  source 1..103: mol_type = other DNA
gcgcgtcctggattcgcggaaacgcgtacatccagctgacgagtcccaaatcggaataccagcatcgtcttgatgcccttggaagtccgaggacgaaacgcgc

SEQ ID NO: 418 - DNA, 105 bp, synthetic construct
  source 1..105: mol_type = other DNA
gcgcgtcctggattcgcggaaacgcgtacatcgagaataccagcatcgtcttgatgcccttggaagtctcgccagctgacgagtcccaaataggacgaaacgcgc

SEQ ID NO: 419 - DNA, 101 bp, synthetic construct
  source 1..101: mol_type = other DNA
gcgcgtcctggattcgcggaaacgcgtacatccagctgacgagtcccaaataggacgaaagaataccagcatcgtcttgatgcccttggaagtctacgcgc

SEQ ID NO: 420 - DNA, 103 bp, synthetic construct
  source 1..103: mol_type = other DNA
gcgcgtcctggattcgcggaaacggagaataccagcatcgtcttgatgcccttggaagtctccgtacatccagctgacgagtcccaaataggacgaaacgcgc

SEQ ID NO: 421 - DNA, 103 bp, synthetic construct
  source 1..103: mol_type = other DNA
gcgcgtcctggattcgcggaaagagaataccagcatcgtcttgatgcccttggaagtctccgcgtacatccagctgacgagtcccaaataggacgaaacgcgc

SEQ ID NO: 422 - DNA, 103 bp, synthetic construct
  source 1..103: mol_type = other DNA
gcgcgtcctggattcgcggaaacgcgtacatccagctgacgagtcccaaataggacgacggaataccagcatcgtcttgatgcccttggaagtccgaacgcgc

SEQ ID NO: 423 - DNA, 105 bp, synthetic construct
  source 1..105: mol_type = other DNA
gcgcgtcctggattcgcggaaacgcgtacatccagctgacgagccggaataccagcatcgtcttgatgcccttggaagtccggtcccaaataggacgaaacgcgc

SEQ ID NO: 424 - DNA, 105 bp, synthetic construct
  source 1..105: mol_type = other DNA
gcgcgtcctggattcgcggaaacgcgtacatccagctgcgagaataccagcatcgtcttgatgcccttggaagtctcgacgagtcccaaataggacgaaacgcgc

SEQ ID NO: 425 - DNA, 101 bp, synthetic construct
  source 1..101: mol_type = other DNA
gcgcgtcctggattcgcggaaacgcgtacaggaataccagcatcgtcttgatgcccttggaagtcctccagctgacgagtcccaaataggacgaaacgcgc

SEQ ID NO: 426 - DNA, 105 bp, synthetic construct
  source 1..105: mol_type = other DNA
gcgcgtcctggattcgcggaaacgcgtacatccagctgacgagtcccaaataggacgccggaataccagcatcgtcttgatgcccttggaagtccggaaacgcgc

SEQ ID NO: 427 - DNA, 105 bp, synthetic construct
  source 1..105: mol_type = other DNA
gcgcgtcctgccggaataccagcatcgtcttgatgcccttggaagtccgggattcgcggaaacgcgtacatccagctgacgagtcccaaataggacgaaacgcgc

SEQ ID NO: 428 - DNA, 103 bp, synthetic construct
  source 1..103: mol_type = other DNA
gcgcgtcctggattcgcggaaacgcgtacatccagctgacgagtccgagaataccagcatcgtcttgatgcccttggaagtctccaaataggacgaaacgcgc

SEQ ID NO: 429 - DNA, 101 bp, synthetic construct
  source 1..101: mol_type = other DNA
gcgcgtcctggattcgcggggaataccagcatcgtcttgatgcccttggaagtccaaacgcgtacatccagctgacgagtcccaaataggacgaaacgcgc

SEQ ID NO: 430 - DNA, 103 bp, synthetic construct
  source 1..103: mol_type = other DNA
gcgcgtcctggattcgcggaaacgcgtacatccagctgagagaataccagcatcgtcttgatgcccttggaagtctccgagtcccaaataggacgaaacgcgc

SEQ ID NO: 431 - DNA, 103 bp, synthetic construct
  source 1..103: mol_type = other DNA
gcgcgtcctggattcgcggaaacgcgtacatccacggaataccagcatcgtcttgatgcccttggaagtccggctgacgagtcccaaataggacgaaacgcgc

SEQ ID NO: 432 - DNA, 105 bp, synthetic construct
  source 1..105: mol_type = other DNA
gcgcgtcctggattcgcggaaacgcgtacatccagctgacgagtcccaccggaataccagcatcgtcttgatgcccttggaagtccggaataggacgaaacgcgc

SEQ ID NO: 433 - DNA, 103 bp, synthetic construct
  source 1..103: mol_type = other DNA
gcgcgtcctggattcgcggaaacgcgtacatccagctgacgagtcccaaataggaagtgataccagcatcgtcttgatgcccttggcagcactcgaaacgcgc

SEQ ID NO: 434 - DNA, 101 bp, synthetic construct
  source 1..101: mol_type = other DNA
gcgcgtccagaataccagcatcgtcttgatgcccttggaagtcttggattcgcggaaacgcgtacatccagctgacgagtcccaaataggacgaaacgcgc

SEQ ID NO: 435 - DNA, 103 bp, synthetic construct
  source 1..103: mol_type = other DNA
gcgcgtcctggattcgcggaaacgcgtacatccagctgacgagtcccaaacggaataccagcatcgtcttgatgcccttggaagtccgtaggacgaaacgcgc

SEQ ID NO: 436 - DNA, 103 bp, synthetic construct
  source 1..103: mol_type = other DNA
gcgcgtcctggattcgcggaaacgcgtacatccagcggaataccagcatcgtcttgatgcccttggaagtccgctgacgagtcccaaataggacgaaacgcgc

SEQ ID NO: 437 - DNA, 101 bp, synthetic construct
  source 1..101: mol_type = other DNA
gcgcgtcctggattcgcggaaacgcgtacatccagctgacgagtcccaagaataccagcatcgtcttgatgcccttggaagtctaataggacgaaacgcgc

SEQ ID NO: 438 - DNA, 103 bp, synthetic construct
  source 1..103: mol_type = other DNA
gcgcgtcctggattcgcggaaacgcgtacatccagctgacgagtcccaaataggacgcggaataccagcatcgtcttgatgcccttggaagtccgaaacgcgc

SEQ ID NO: 439 - DNA, 103 bp, synthetic construct
  source 1..103: mol_type = other DNA
gcgcgtgagaataccagcatcgtcttgatgcccttggaagtctccctggattcgcggaaacgcgtacatccagctgacgagtcccaaataggacgaaacgcgc

SEQ ID NO: 440 - DNA, 103 bp, synthetic construct
  source 1..103: mol_type = other DNA
gcgcgtcctggattcgcggaaacgcgtacatccagctgacgagtcccaaataggacgaaacgccggaataccagcatcgtcttgatgcccttggaagtccggc

SEQ ID NO: 441 - DNA, 105 bp, synthetic construct
  source 1..105: mol_type = other DNA
gcgcccggaataccagcatcgtcttgatgcccttggaagtccgggtcctggattcgcggaaacgcgtacatccagctgacgagtcccaaataggacgaaacgcgc

SEQ ID NO: 442 - DNA, 103 bp, synthetic construct
  source 1..103: mol_type = other DNA
gcgcgtcctggattcgcggaaacgcgtacatccggaataccagcatcgtcttgatgcccttggaagtccgcagctgacgagtcccaaataggacgaaacgcgc

SEQ ID NO: 443 - DNA, 103 bp, synthetic construct
  source 1..103: mol_type = other DNA
gcgcgtcctggattcgcggaaacgcgtacatccagctgacgagtcccaaataggacggaataccagcatcgtcttgatgcccttggaagtccgcgaaacgcgc

SEQ ID NO: 444 - DNA, 105 bp, synthetic construct
  source 1..105: mol_type = other DNA
gcgcgtcccggaataccagcatcgtcttgatgcccttggaagtccggctggattcgcggaaacgcgtacatccagctgacgagtcccaaataggacgaaacgcgc

SEQ ID NO: 445 - DNA, 103 bp, synthetic construct
  source 1..103: mol_type = other DNA
gcgcgtcgagaataccagcatcgtcttgatgcccttggaagtctcctggattcgcggaaacgcgtacatccagctgacgagtcccaaataggacgaaacgcgc

SEQ ID NO: 446 - DNA, 103 bp, synthetic construct
  source 1..103: mol_type = other DNA
gcgcgtcctggattccggaataccagcatcgtcttgatgcccttggaagtccggcggaaacgcgtacatccagctgacgagtcccaaataggacgaaacgcgc

SEQ ID NO: 447 - DNA, 101 bp, synthetic construct
  source 1..101: mol_type = other DNA
gcgcgtcctggattcgcggaaacgcgtacatccagctgacgagtccggaataccagcatcgtcttgatgcccttggaagtcccaaataggacgaaacgcgc

SEQ ID NO: 448 - DNA, 103 bp, synthetic construct
  source 1..103: mol_type = other DNA
gcgcgcggaataccagcatcgtcttgatgcccttggaagtccgtcctggattcgcggaaacgcgtacatccagctgacgagtcccaaataggacgaaacgcgc

SEQ ID NO: 449 - DNA, 103 bp, synthetic construct
  source 1..103: mol_type = other DNA
gcgcgtcctggattcgcggaaacgcgtacatccagctgacgagtcccaaataggacgaaacgcgagtgataccagcatcgtcttgatgcccttggcagcactc

SEQ ID NO: 450 - DNA, 105 bp, synthetic construct
  source 1..105: mol_type = other DNA
gcgcgtcctggattcgcggaaacgcgtacatccagctgacgagtcccaaataggacgaaacccggaataccagcatcgtcttgatgcccttggaagtccgggcgc

SEQ ID NO: 451 - DNA, 101 bp, synthetic construct
  source 1..101: mol_type = other DNA
gcgcgtcctggattcgcggaagaataccagcatcgtcttgatgcccttggaagtctaacgcgtacatccagctgacgagtcccaaataggacgaaacgcgc

SEQ ID NO: 452 - DNA, 101 bp, synthetic construct
  source 1..101: mol_type = other DNA
gcgcgtcctggagaataccagcatcgtcttgatgcccttggaagtctattcgcggaaacgcgtacatccagctgacgagtcccaaataggacgaaacgcgc

SEQ ID NO: 453 - DNA, 101 bp, synthetic construct
  source 1..101: mol_type = other DNA
gcgcgtcctggattcgcggaaacgcgtacatccagctgacgagtcccaaataggacgaaacagaataccagcatcgtcttgatgcccttggaagtctgcgc

SEQ ID NO: 454 - DNA, 101 bp, synthetic construct
  source 1..101: mol_type = other DNA
gcgcgtcctggattcgcggaaacgcgtacatccagctgacgagtccagaataccagcatcgtcttgatgcccttggaagtctcaaataggacgaaacgcgc

SEQ ID NO: 455 - DNA, 105 bp, synthetic construct
  source 1..105: mol_type = other DNA
gcgcgtcctggattcgcggaaacgcgtacatccagctgacgagtccccggaataccagcatcgtcttgatgcccttggaagtccggcaaataggacgaaacgcgc

SEQ ID NO: 456 - DNA, 105 bp, synthetic construct
  source 1..105: mol_type = other DNA
gcgcgtcctggattcgcggaaacgcgtacatccagctgacgagtcccaaataccggaataccagcatcgtcttgatgcccttggaagtccggggacgaaacgcgc

SEQ ID NO: 457 - DNA, 103 bp, synthetic construct
  source 1..103: mol_type = other DNA
gcgcgtcctggattcgcggaaacgcgtacatccagctgacgagtcccaaataggacgaagtgataccagcatcgtcttgatgcccttggcagcactaacgcgc

SEQ ID NO: 458 - DNA, 105 bp, synthetic construct
  source 1..105: mol_type = other DNA
gcgcgtcctggattcgcggaaacgcgtacatccagctgacgagtcccaaataggacgaaccggaataccagcatcgtcttgatgcccttggaagtccggacgcgc

SEQ ID NO: 459 - DNA, 101 bp, synthetic construct
  source 1..101: mol_type = other DNA
gcgcgtcctggattcgcggaaacgcgtacatccagctgacgagtcccaaataggacgaaggaataccagcatcgtcttgatgcccttggaagtccacgcgc

SEQ ID NO: 460 - DNA, 103 bp, synthetic construct
  source 1..103: mol_type = other DNA
gcgcgtcctggattcgcagtgataccagcatcgtcttgatgcccttggcagcactggaaacgcgtacatccagctgacgagtcccaaataggacgaaacgcgc

SEQ ID NO: 461 - DNA, 103 bp, synthetic construct
  source 1..103: mol_type = other DNA
gcgcgtcctggattcgcggaaacgcgtacatccaggagaataccagcatcgtcttgatgcccttggaagtctcctgacgagtcccaaataggacgaaacgcgc

SEQ ID NO: 462 - DNA, 105 bp, synthetic construct
  source 1..105: mol_type = other DNA
gcgcgtcctggattcgcgccggaataccagcatcgtcttgatgcccttggaagtccgggaaacgcgtacatccagctgacgagtcccaaataggacgaaacgcgc

SEQ ID NO: 463 - DNA, 105 bp, synthetic construct
  source 1..105: mol_type = other DNA
gcgcgtcctggattcgcggaaacgcgtacatccagctgacgagtcgagaataccagcatcgtcttgatgcccttggaagtctcgcccaaataggacgaaacgcgc

SEQ ID NO: 464 - DNA, 103 bp, synthetic construct
  source 1..103: mol_type = other DNA
gcgcgtcctggagtgataccagcatcgtcttgatgcccttggcagcactattcgcggaaacgcgtacatccagctgacgagtcccaaataggacgaaacgcgc

SEQ ID NO: 465 - DNA, 105 bp, synthetic construct
  source 1..105: mol_type = other DNA
gcgcgtcctggattcgcggaaacgcgtacatccagctgaccgagaataccagcatcgtcttgatgcccttggaagtctcggagtcccaaataggacgaaacgcgc

SEQ ID NO: 466 - DNA, 103 bp, synthetic construct
  source 1..103: mol_type = other DNA
gcgcgtcctggattcgcggaacggaataccagcatcgtcttgatgcccttggaagtccgacgcgtacatccagctgacgagtcccaaataggacgaaacgcgc

SEQ ID NO: 467 - DNA, 101 bp, synthetic construct
  source 1..101: mol_type = other DNA
gcgcgtcctggattcgcggaaacgagaataccagcatcgtcttgatgcccttggaagtctcgtacatccagctgacgagtcccaaataggacgaaacgcgc

SEQ ID NO: 468 - DNA, 105 bp, synthetic construct
  source 1..105: mol_type = other DNA
gcgcgtcctggattcgcggaaacgcgtacatccagccgagaataccagcatcgtcttgatgcccttggaagtctcgtgacgagtcccaaataggacgaaacgcgc

SEQ ID NO: 469 - DNA, 105 bp, synthetic construct
  source 1..105: mol_type = other DNA
gcgagaataccagcatcgtcttgatgcccttggaagtctcgcgcgtcctggattcgcggaaacgcgtacatccagctgacgagtcccaaataggacgaaacgcgc

SEQ ID NO: 470 - DNA, 105 bp, synthetic construct
  source 1..105: mol_type = other DNA
gcgcgtcctggattcgcggaaaccgagaataccagcatcgtcttgatgcccttggaagtctcggcgtacatccagctgacgagtcccaaataggacgaaacgcgc

SEQ ID NO: 471 - DNA, 105 bp, synthetic construct
  source 1..105: mol_type = other DNA
gcgcgtcctggattcgcggaaacgcgtacatccagctgacgagtcccaaataggaccgagaataccagcatcgtcttgatgcccttggaagtctcggaaacgcgc

SEQ ID NO: 472 - DNA, 103 bp, synthetic construct
  source 1..103: mol_type = other DNA
gcgcgtcctggattcgcggaaacgcgtacatccagctgacgagtcccaaatagagaataccagcatcgtcttgatgcccttggaagtctcggacgaaacgcgc

SEQ ID NO: 473 - DNA, 103 bp, synthetic construct
  source 1..103: mol_type = other DNA
gcgcgtcctggattcgcggaaacgcgtacatccagctgacgagtcccaaatagtgataccagcatcgtcttgatgcccttggcagcactaggacgaaacgcgc

SEQ ID NO: 474 - DNA, 103 bp, synthetic construct
  source 1..103: mol_type = other DNA
gcgcgtcctggattcgcggaaacgcgtacatccagctgacgagtcccaaataggacgaaacgcgcggaataccagcatcgtcttgatgcccttggaagtccgc

SEQ ID NO: 475 - DNA, 103 bp, synthetic construct
  source 1..103: mol_type = other DNA
gcgcgtcctggattcgcggaaacgcgtacatccagctgacgagtcccaaataggacgaaacgcgagaataccagcatcgtcttgatgcccttggaagtctcgc

SEQ ID NO: 476 - DNA, 101 bp, synthetic construct
  source 1..101: mol_type = other DNA
gcgcgtcctggattcgcggaaacgcgtacatccagctgacgagtcccaaataggacgaaaagaataccagcatcgtcttgatgcccttggaagtctcgcgc

SEQ ID NO: 477 - DNA, 52 bp, synthetic construct; identical to SEQ ID NO: 215
  source 1..52: mol_type = other DNA

SEQ ID NO: 478 - DNA, 76 bp, synthetic construct; identical to SEQ ID NO: 262
  source 1..76: mol_type = other DNA

SEQ ID NO: 479 - DNA, 83 bp, synthetic construct
  source 1..83: mol_type = other DNA
ctgaggtgcaggtacatccagctgacgagtcccaaataggacgaaacgcgcttcggtgcgtcctggattccactgctatccac

SEQ ID NO: 480 - DNA, 111 bp, synthetic construct
  source 1..111: mol_type = other DNA
ctgaggtgcaggtacatccagctgacgagtcccaaataggacgaaacgcggaataccagcatcgtcttgatgcccttggaagtccgcgtcctggattccactgctatccac

SEQ ID NO: 481 - DNA, 64 bp, synthetic construct
  source 1..64: mol_type = other DNA
gggagccctgtcaccggatgtgctttccggtctgatgagtccgtgaggacgaaacagggctccc

SEQ ID NO: 482 - DNA, 54 bp, synthetic construct
  source 1..54: mol_type = other DNA
gcgcgtcaccggatgtgctttccggtctgatgagtccgtgaggacgaaacgcgc

SEQ ID NO: 483 - DNA, 77 bp, synthetic construct
  source 1..77: mol_type = other DNA
gcgcgtcaccggaataccagcatcgtcttgatgcccttggaagtccggtctgatgagtccgtgaggacgaaacgcgc

SEQ ID NO: 484 - DNA, 65 bp, synthetic construct; identical to SEQ ID NO: 113
  source 1..65: mol_type = other DNA

SEQ ID NO: 485 - DNA, 97 bp, synthetic construct
  source 1..97: mol_type = other DNA
gcgcgtcctggattcgcgggaataccagcatcgtcttgatgcccttggaagtcccgcgtacatccagctgacgagtcccaaataggacgaaacgcgc

SEQ ID NO: 486 - DNA, 95 bp, synthetic construct
  source 1..95: mol_type = other DNA
gcgcgtcctggattcgagtgataccagcatcgtcttgatgcccttggcagcactcgtacatccagctgacgagtcccaaataggacgaaacgcgc

SEQ ID NO: 487 - DNA, 137 bp, synthetic construct
  source 1..137: mol_type = other DNA
gcgcgtcctggattcgcggaaacgcgtacatccagctgacgagtcccaaataggacgaaacgcgctagtactgttctgtacgtgcgcgtcaccggatgtgctttccggtctgatgagtccgtgaggacgaaacgcgc

SEQ ID NO: 488 - DNA, 36 bp, synthetic construct
  source 1..36: mol_type = other DNA
ggaataccagcatcgtcttgatgcccttggaagtcc

SEQ ID NO: 489 - DNA, 38 bp, synthetic construct
  source 1..38: mol_type = other DNA
cggaataccagcatcgtcttgatgcccttggaagtccg

SEQ ID NO: 490 - DNA, 40 bp, synthetic construct
  source 1..40: mol_type = other DNA
ccggaataccagcatcgtcttgatgcccttggaagtccgg

SEQ ID NO: 491 - DNA, 36 bp, synthetic construct
  source 1..36: mol_type = other DNA
agaataccagcatcgtcttgatgcccttggaagtct

SEQ ID NO: 492 - DNA, 38 bp, synthetic construct
  source 1..38: mol_type = other DNA
gagaataccagcatcgtcttgatgcccttggaagtctc

SEQ ID NO: 493 - DNA, 40 bp, synthetic construct
  source 1..40: mol_type = other DNA
cgagaataccagcatcgtcttgatgcccttggaagtctcg

SEQ ID NO: 494 - DNA, 38 bp, synthetic construct
  source 1..38: mol_type = other DNA
agtgataccagcatcgtcttgatgcccttggcagcact

SEQ ID NO: 495 - DNA, 2214 bp, synthetic construct
  source 1..2214: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 496 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 497 - protein, 601 aa, synthetic construct
TAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 498 - protein, 535 aa, synthetic construct
MAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

(19) United States
(12) Patent Application Publication
(10) Pub. No.: US 2026/0167959 A1
(43) Pub. Date: Jun. 18, 2026

(54) APTAZYME-BASED REGULATABLE GENE EXPRESSION SYSTEMS

(71) Applicant: REJUVENATE BIO INC, San Diego, CA (US)

(72) Inventors: Noah Davidsohn, Brookline, MA (US); Louis Metzger, San Diego, CA (US); Rokib Hasan, San Diego, CA (US)

(21) Appl. No.: 19/125,842

(22) PCT Filed: Nov. 3, 2023
(86) PCT No.: PCT/US2023/078621

§ 371 Date: Apr. 30, 2025

Related U.S. Application Data
(60) Provisional application No. 63/382,165, filed on Nov. 3, 2022.

(51) Int. Cl. C12N 15/113; A61K 31/4545; A61K 31/522; A61K 48/00; C12N 15/86

(52) U.S. Cl. CPC C12N 15/113; A61K 31/4545; A61K 31/522; C12N 15/86; A61K 48/00; C12N 2310/12; C12N 2750/14143; C12N 2750/14152

(57) ABSTRACT

Provided herein are ligand responsive ribozymes comprising a ligand responsive aptamer linked to a self-cleaving ribozyme for the ligand-dependent regulation of gene expression and methods of use thereof.

### RELATED APPLICATIONS

This application claims priority to U.S. Provisional Patent Application Ser. No. 63/382,165, filed Nov. 3, 2022, the entire disclosure of which is hereby incorporated herein by reference.

### REFERENCE TO SEQUENCE LISTING

This application contains a sequence listing which has been submitted electronically in ST.26 format and is hereby incorporated by reference in its entirety (said ST.26 copy, created on Oct. 31, 2023, is named “203433_seqlist.xml” and is 484,394 bytes in size).

### BACKGROUND

Regulatable gene expression systems are crucial for various therapeutic approaches including, but not limited to, gene therapy. One type of system is based on the incorporation of aptazymes that allows for conditional regulation of gene expression. Aptazymes are fusions of a ligand-binding RNA aptamer and a self-cleaving ribozyme. Conditional cleavage is mediated by binding of a specific ligand by the aptamer domain and subsequent conformational changes in riboswitch architecture. Such aptazymes can be encoded into DNA, e.g., in the 3′ untranslated region (UTR) upstream of a polyadenylation signal of an expression construct to conditionally cleave the mRNA transcript. Self-cleavage within the 3′ UTR can be exploited for the post-transcriptional control of gene expression by decreasing mRNA stability via conditional poly(A) tail cleavage. These properties of aptazymes make them attractive for use in a wide range of applications in biology and medicines. However, identifying aptazymes that function efficiently in mammalian systems remains challenging, at least due to the difficulties associated with generating and screening aptazymes that function in the cellular environment, leaky expression, and narrow dynamic ranges.

Accordingly, there is a need in the art for novel aptazyme-based regulatable gene expression systems for use in gene therapy applications.

### SUMMARY

Provided herein are ligand responsive ribozymes comprising a ligand responsive aptamer linked to a self-cleaving ribozyme for the ligand-dependent regulation of gene expression, and methods of use thereof.

Accordingly, in one aspect, the present disclosure provides a polynucleotide comprising a transgene operably linked to a ligand responsive ribozyme, wherein the ligand responsive ribozyme comprises a nucleic acid sequence having at least 85% sequence identity to the sequence set forth in any one of SEQ ID NOs: 1-50.

In certain embodiments, the ligand responsive ribozyme is inserted into a 3′ untranslated region (UTR) and/or 5′ UTR of the transgene.

In certain embodiments, the ligand responsive ribozyme is responsive to theophylline or a derivative thereof. In certain embodiments, the theophylline derivative is aminophylline or dyphylline.

In certain embodiments, the transgene encodes one or more polypeptides. In certain embodiments, the transgene encodes a miRNA, shRNA, siRNA, antisense RNA, gRNA, antagomir, miRNA sponge, RNA aptazyme, RNA aptamer, lncRNA, ribozyme or mRNA.

In certain embodiments, the transgene is operably linked to a transcriptional regulatory element.

In another aspect, the present disclosure provides a vector comprising a polynucleotide as described herein.

In certain embodiments, the vector is a plasmid, a viral vector, or a DNA minimal vector. In certain embodiments, the vector is an expression vector.

In certain embodiments, the viral vector is selected from the group consisting of adenoviral vector, adeno-associated virus (AAV) vector, and lentiviral vector. In certain embodiments, the vector is an AAV vector.

In another aspect, the present disclosure provides a ligand responsive ribozyme comprising a nucleic acid sequence having at least 85% identity to the sequence set forth in any one of SEQ ID NOs: 1-50.

In another aspect, the present disclosure provides a recombinant AAV (rAAV) genome comprising a polynucleotide as described herein.

In certain embodiments, the rAAV genome further comprises a 5′ inverted terminal repeat (5′ ITR) sequence, and a 3′ inverted terminal repeat (3′ ITR) sequence.

In certain embodiments, the rAAV genome is a single stranded rAAV genome. In certain embodiments, the rAAV genome is a self-complementary rAAV genome.

In another aspect, the present disclosure provides a recombinant cell comprising a polynucleotide as described herein, a vector as described herein, a ribozyme as described herein, or a rAAV genome as described herein.

In another aspect, the present disclosure provides a recombinant adeno-associated virus (rAAV) comprising: (a) a capsid comprising an AAV capsid protein; and (b) a rAAV genome as described herein.

In certain embodiments, the AAV capsid protein is derived from a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein, or an engineered variant thereof.

In another aspect, the present disclosure provides a polynucleotide as described herein, a vector as described herein, or a rAAV as described herein, for use in medicine.

In another aspect, the present disclosure provides a polynucleotide as described herein, a vector as described herein, or a rAAV as described herein, for use as therapy.

In another aspect, the present disclosure provides a polynucleotide as described herein, a vector as described herein, or a rAAV as described herein, for use as a medicament.

In another aspect, the present disclosure provides a packaging system for preparation of an rAAV, wherein the packaging system comprises: (a) a first nucleotide sequence encoding one or more AAV Rep proteins; (b) a second nucleotide sequence encoding an AAV capsid protein; and (c) a third nucleotide sequence comprising the rAAV genome sequence as described herein.

In certain embodiments, the packaging system comprises a first vector comprising the first nucleotide sequence and the second nucleotide sequence, and a second vector comprising the third nucleotide sequence.

In certain embodiments, the packaging system further comprises a fourth nucleotide sequence comprising one or more helper virus genes, optionally wherein the fourth nucleotide sequence is comprised within a third vector. In certain embodiments, the fourth nucleotide sequence comprises one or more genes from a virus selected from the group consisting of adenovirus, herpesvirus, vaccinia virus, and cytomegalovirus (CMV).

In certain embodiments, the first vector, second vector, and/or the third vector is a plasmid.

In another aspect, the present disclosure provides a method for recombinant preparation of an rAAV, the method comprising introducing a packaging system as described herein into a cell under conditions whereby the rAAV is produced.

In another aspect, the present disclosure provides a method comprising introducing into a cell a polynucleotide as described herein, a vector as described herein, or a rAAV genome as described herein; or transducing a cell with a rAAV as described herein.

In another aspect, the present disclosure provides a method of modulating the expression of a transgene comprising: (a) introducing into a cell a polynucleotide as described herein, a vector as described herein, or a rAAV genome as described herein; or transducing a cell with a rAAV as described herein; and (b) contacting the cell of (a) with an effective amount of theophylline or a derivative thereof.

In certain embodiments, contacting the cell of (a) with an effective amount of the theophylline or the derivative thereof increases expression of the transgene.

In certain embodiments, the theophylline derivative is aminophylline or dyphylline.

In certain embodiments, the cell is in a subject and the polynucleotide, vector, or rAAV is administered to the subject.

In certain embodiments, the cell is a muscle cell and/or a liver cell.

In certain embodiments, the polynucleotide, vector, or rAAV is administered to the subject intravenously, intraperitoneally, subcutaneously, intramuscularly, intrathecally, or intradermally.

In another aspect, the present disclosure provides a method of modulating the expression of a transgene in a subject, comprising administering theophylline or a derivative thereof to the subject at an amount effect to induce expression of the transgene, wherein the transgene is comprised within a polynucleotide as described herein, a vector as described herein, or a rAAV genome as described herein.

In certain embodiments, the transgene further comprises a suicide gene. In certain embodiments, the suicide gene is Herpes Simplex Virus Thymidine Kinase (HSV-TK) or inducible Caspase 9 (iCasp9).

In certain embodiments, the method further comprises administering an agent to the subject at an amount effective to ablate the recombinant cell. In certain embodiments, the agent is ganciclovir or AP20187.

In another aspect, the present disclosure provides a method of modulating the expression of a transgene in a subject, comprising administering theophylline or a derivative thereof to the subject at an amount effect to induce expression of the transgene, wherein the transgene is comprised within a recombinant cell as described herein, and the recombinant cell has been administered to the subject.

In certain embodiments, the recombinant cell is an immune cell, optionally an immune cell autologous to the subject. In certain embodiments, the immune cell is a T cell, NK cell, NKT cell, or precursor thereof.

In certain embodiments, the polynucleotide is integrated into a genomic locus of the recombinant cell. In certain embodiments, the genomic locus is a safe harbor locus.

In certain embodiments, the transgene encodes an exogenous T cell receptor (TCR) and/or chimeric antigen receptor (CAR).

In certain embodiments, the transgene further comprises a suicide gene. In certain embodiments, the suicide gene is Herpes Simplex Virus Thymidine Kinase (HSV-TK) or inducible Caspase 9 (iCasp9).

In certain embodiments, the method further comprises administering an agent to the subject at an amount effective to ablate the recombinant cell. In certain embodiments, the agent is ganciclovir or AP20187.

In certain embodiments, the subject is selected from the group consisting of a human subject, a canine subject, a feline subject, and an equine subject.

### BRIEF DESCRIPTION OF THE DRAWINGS

FIG. 1 is a graph depicting the expression of luciferase detected from cells comprising the indicated aptazyme constructs, cultured in DMEM media with or without 1 mM theophylline.

FIG. 2 is a graph depicting the fold-change in luciferase expression detected from cells comprising an aptazyme construct cultured in the presence of theophylline, over luciferase expression detected from cells comprising the same aptazyme construct cultured in the absence of theophylline. The lines on the x-axis represent the fold-change in luciferase expression detected from cells comprising each unique aptazyme construct generated in the aptazyme variant library described in Example 2.

FIG. 3 is a graph depicting luciferase expression as a function of barcode read counts (see, Example 2) detected from cells comprising an exemplary aptazyme construct RA004 in the presence or absence of theophylline (“inducer”).

FIG. 4 is a graph depicting GFP expression detected from cells comprising the aptazymes C11, BFRA328, and RA210, in the presence or absence of theophylline. Negative control was cells comprising only the ribozyme sequence, and positive control was cells that did not contain any aptazyme.

### DETAILED DESCRIPTION

Provided herein are ligand responsive ribozymes comprising a ligand responsive aptamer linked to a self-cleaving ribozyme for the ligand-dependent regulation of gene expression and methods of use thereof. The ligand responsive ribozymes described herein have been isolated from screening an aptazyme library containing unique aptazyme sequences for expression in the presence and absence of ligand (e.g., theophylline).

### I. Definitions

As used herein, the term “AAV” is a standard abbreviation for adeno-associated virus.

As used herein interchangeably, the terms “recombinant adeno-associated virus” or “rAAV” refer to an AAV comprising a genome lacking functional rep and cap genes.

As used herein, the term “cap gene” refers to a nucleic acid sequence that encodes a capsid protein. For AAV, the capsid protein may be VP1, VP2, or VP3. VP1, VP2, and/or VP3 capsid proteins assemble into a capsid that surrounds the rAAV genome.

As used herein, the term “rep gene” refers to the nucleic acid sequences that encode the non-structural proteins (e.g., rep78, rep68, rep52, and rep40) required for the replication and production of an AAV.

As used herein, the term “rAAV genome” refers to a nucleic acid molecule (e.g., DNA and/or RNA) comprising the genome sequence of an rAAV. The skilled artisan will appreciate that where an rAAV genome comprises a transgene (e.g., a polypeptide encoding a therapeutic protein operably linked to a transcriptional regulatory element (i.e., payload)), the rAAV genome can be in the sense or antisense orientation relative to the direction of transcription of the transgene.

As used herein, an “isolated polynucleotide” refers to a polynucleotide that has been separated from one or more nucleic acid molecules present in the natural source of the polynucleotide.

As used herein, the “percentage identity” between two nucleotide sequences or between two amino acid sequences is calculated by multiplying the number of matches between the pair of aligned sequences by 100, and dividing by the length of the aligned region, including internal gaps. Identity scoring only counts perfect matches, and does not consider the degree of similarity of amino acids to one another. Note that only internal gaps are included in the length, not gaps at the sequence ends.

As used herein, a “vector” refers to a nucleic acid molecule that is a vehicle for introducing a nucleic acid molecule (e.g., a polynucleotide described herein) into a cell.

As used herein, an “expression vector” refers to a vector comprising transcriptional regulatory elements operably linked to a gene of interest (e.g., a polynucleotide described herein) that facilitate the expression of the gene of interest in a cell and/or a cell free expression system.

As used herein, the term “transgene” refers to a non-AAV nucleic acid sequence that encodes a polypeptide (e.g., an antibody or scFv) or non-coding RNA (e.g., an miRNA, shRNA, siRNA, antisense RNA, gRNA, antagomir, miRNA sponge, RNA aptazyme, or RNA aptamer).

As used herein, the term “transcriptional regulatory element” or “TRE” refers to a cis-acting nucleotide sequence, for example, a DNA sequence, that regulates (e.g., controls, increases, or reduces) transcription of an operably linked nucleotide sequence by an RNA polymerase to form an RNA molecule. A TRE may comprise one or more promoter elements and/or enhancer elements. A skilled artisan would appreciate that the promoter and enhancer elements in a gene may be close in location, and the term “promoter” may refer to a sequence comprising a promoter element and an enhancer element. Thus, the term “promoter” does not exclude an enhancer element in the sequence. The promoter and enhancer elements do not need to be derived from the same gene or species, and the sequence of each promoter or enhancer element may be either identical or substantially identical to the corresponding endogenous sequence in the genome.

As used herein, the term “operably linked” is used to describe the connection between a TRE and a polynucleotide sequence (e.g., a transgene described herein) to be transcribed. Typically, gene expression is placed under the control of a TRE comprising one or more promoter and/or enhancer elements. The transgene is “operably linked” to the TRE if the transcription of the transgene is controlled or influenced by the TRE. The promoter and enhancer elements of the TRE may be in any orientation and/or distance from the transgene, as long as the desired transcriptional activity is obtained. In an embodiment, the TRE is upstream from the transgene.

As used herein, the term “effective amount” in the context of the contact of a cell with theophylline refers to the amount of the theophylline that achieves a desired effect (e.g., binds to an aptazyme and allows for transgene expression).

As used herein, the term “suicide gene” refers to a gene that is capable of causing a cell expressing the gene to die. In certain embodiments, a suicide gene causes death of a cell expressing the suicide gene only when the cell is exposed to a specific agent (e.g., a drug).

### II. Polynucleotides, Vectors, and Compositions

In one aspect, the present disclosure provides nucleic acid sequences that encode ligand responsive ribozymes that have been shown to result in tight control of gene expression in the OFF-state (i.e., when unbound to ligand), and a dynamic range of enhanced expression in the ON-state (i.e., when bound to ligand), of a transgene operably linked thereto. As described in the examples, the ligand responsive ribozymes described herein were identified by screening a library of ligand responsive ribozymes generated by inserting a ligand responsive aptamer after each base pair of a ribozyme. This base-by-base insertion frameshift screening of ligand responsive ribozymes creates unique aptazyme structures.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 1. In certain embodiments, the nucleic acid sequence comprises or consists of SEQ ID NO: 1.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 2. In certain embodiments, the nucleic acid sequence comprises or consists of SEQ ID NO: 2.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 3. In certain embodiments, the nucleic acid sequence comprises or consists of SEQ ID NO: 3.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 4. In certain embodiments, the nucleic acid sequence comprises or consists of SEQ ID NO: 4.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 5. In certain embodiments, the nucleic acid sequence comprises or consists of SEQ ID NO: 5.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 6. In certain embodiments, the nucleic acid sequence comprises or consists of SEQ ID NO: 6.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 7. In certain embodiments, the nucleic acid sequence comprises or consists of SEQ ID NO: 7.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 8. In certain embodiments, the nucleic acid sequence comprises or consists of SEQ ID NO: 8.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 9. In certain embodiments, the nucleic acid sequence comprises or consists of SEQ ID NO: 9.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 10. In certain embodiments, the nucleic acid sequence comprises or consists of SEQ ID NO: 10.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 11. In certain embodiments, the nucleic acid sequence comprises or consists of SEQ ID NO: 11.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 12. In certain embodiments, the nucleic acid sequence comprises or consists of SEQ ID NO: 12.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 13. In certain embodiments, the nucleic acid sequence comprises or consists of SEQ ID NO: 13.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 14. In certain embodiments, the nucleic acid sequence comprises or consists of SEQ ID NO: 14.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 15. In certain embodiments, the nucleic acid sequence comprises or consists of SEQ ID NO: 15.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 16. In certain embodiments, the nucleic acid sequence comprises or consists of SEQ ID NO: 16.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 17. In certain embodiments, the nucleic acid sequence comprises or consists of SEQ ID NO: 17.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 18. In certain embodiments, the nucleic acid sequence comprises or consists of SEQ ID NO: 18.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 19. In certain embodiments, the nucleic acid sequence comprises or consists of SEQ ID NO: 19.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 20. In certain embodiments, the nucleic acid sequence comprises or consists of SEQ ID NO: 20.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 21. In certain embodiments, the nucleic acid sequence comprises or consists of SEQ ID NO: 21.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 22. In certain embodiments, the nucleic acid sequence comprises or consists of SEQ ID NO: 22.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 23. In certain embodiments, the nucleic acid sequence comprises or consists of SEQ ID NO: 23.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 24. In certain embodiments, the nucleic acid sequence comprises or consists of SEQ ID NO: 24.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 25. In certain embodiments, the nucleic acid sequence comprises or consists of SEQ ID NO: 25.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 26. In certain embodiments, the nucleic acid sequence comprises or consists of SEQ ID NO: 26.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 27. In certain embodiments, the nucleic acid sequence comprises or consists of SEQ ID NO: 27.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 28. In certain embodiments, the nucleic acid sequence comprises or consists of SEQ ID NO: 28.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 29. In certain embodiments, the nucleic acid sequence comprises or consists of SEQ ID NO: 29.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 30. In certain embodiments, the nucleic acid sequence comprises or consists of SEQ ID NO: 30.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 31. In certain embodiments, the nucleic acid sequence comprises or consists of SEQ ID NO: 31.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 32. In certain embodiments, the nucleic acid sequence comprises or consists of SEQ ID NO: 32.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 33. In certain embodiments, the nucleic acid sequence comprises or consists of SEQ ID NO: 33.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 34. In certain embodiments, the nucleic acid sequence comprises or consists of SEQ ID NO: 34.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 35. In certain embodiments, the nucleic acid sequence comprises or consists of SEQ ID NO: 35.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 36. In certain embodiments, the nucleic acid sequence comprises or consists of SEQ ID NO: 36.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 37. In certain embodiments, the nucleic acid sequence comprises or consists of SEQ ID NO: 37.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 38. In certain embodiments, the nucleic acid sequence comprises or consists of SEQ ID NO: 38.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 39. In certain embodiments, the nucleic acid sequence comprises or consists of SEQ ID NO: 39.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 40. In certain embodiments, the nucleic acid sequence comprises or consists of SEQ ID NO: 40.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 41. In certain embodiments, the nucleic acid sequence comprises or consists of SEQ ID NO: 41.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 42. In certain embodiments, the nucleic acid sequence comprises or consists of SEQ ID NO: 42.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 43. In certain embodiments, the nucleic acid sequence comprises or consists of SEQ ID NO: 43.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 44. In certain embodiments, the nucleic acid sequence comprises or consists of SEQ ID NO: 44.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 45. In certain embodiments, the nucleic acid sequence comprises or consists of SEQ ID NO: 45.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 46. In certain embodiments, the nucleic acid sequence comprises or consists of SEQ ID NO: 46.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 47. In certain embodiments, the nucleic acid sequence comprises or consists of SEQ ID NO: 47.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 48. In certain embodiments, the nucleic acid sequence comprises or consists of SEQ ID NO: 48.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 49. In certain embodiments, the nucleic acid sequence comprises or consists of SEQ ID NO: 49.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in SEQ ID NO: 50. In certain embodiments, the nucleic acid sequence comprises or consists of SEQ ID NO: 50.

In certain embodiments, the nucleic acid sequence has at least 85%, at least 86%, at least 87%, at least 88%, at least 89%, at least 90%, at least 91, at least 92%, at least 93%, at least 94%, at least 95%, at least 96%, at least 97%, at least 98%, or at least 99%, sequence identity to the sequence set forth in any one of SEQ ID NOs: 51-476. In certain embodiments, the nucleic acid sequence comprises or consists of any one of SEQ ID NOs: 51-476.

In certain embodiments, the nucleic acid has at least 85% sequence identity to the sequence set forth in any one of SEQ ID NOs: 1-476. In certain embodiments, the nucleic acid has at least 86% sequence identity to the sequence set forth in any one of SEQ ID NOs: 1-476. In certain embodiments, the nucleic acid has at least 87% sequence identity to the sequence set forth in any one of SEQ ID NOs: 1-476. In certain embodiments, the nucleic acid has at least 88% sequence identity to the sequence set forth in any one of SEQ ID NOs: 1-476. In certain embodiments, the nucleic acid has at least 89% sequence identity to the sequence set forth in any one of SEQ ID NOs: 1-476. In certain embodiments, the nucleic acid has at least 90% sequence identity to the sequence set forth in any one of SEQ ID NOs: 1-476. In certain embodiments, the nucleic acid has at least 91% sequence identity to the sequence set forth in any one of SEQ ID NOs: 1-476. In certain embodiments, the nucleic acid has at least 92% sequence identity to the sequence set forth in any one of SEQ ID NOs: 1-476. In certain embodiments, the nucleic acid has at least 93% sequence identity to the sequence set forth in any one of SEQ ID NOs: 1-476. In certain embodiments, the nucleic acid has at least 94% sequence identity to the sequence set forth in any one of SEQ ID NOs: 1-476. In certain embodiments, the nucleic acid has at least 95% sequence identity to the sequence set forth in any one of SEQ ID NOs: 1-476. In certain embodiments, the nucleic acid has at least 96% sequence identity to the sequence set forth in any one of SEQ ID NOs: 1-476. In certain embodiments, the nucleic acid has at least 97% sequence identity to the sequence set forth in any one of SEQ ID NOs: 1-476. In certain embodiments, the nucleic acid has at least 98% sequence identity to the sequence set forth in any one of SEQ ID NOs: 1-476. In certain embodiments, the nucleic acid has at least 99% sequence identity to the sequence set forth in any one of SEQ ID NOs: 1-476. In certain embodiments, the nucleic acid comprises a sequence set forth in any one of SEQ ID NOs: 1-476. In certain embodiments, the nucleic acid consists of a sequence set forth in any one of SEQ ID NOs: 1-476.

In another aspect, the present disclosure provides polynucleotides comprising a transgene operably linked to the ligand responsive ribozymes described herein.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 1. In certain embodiments, the polynucleotide comprises a transgene operably linked to a ligand responsive ribozyme, wherein the ligand responsive ribozyme comprises or consists of the sequence set forth in SEQ ID NO: 1.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 2. In certain embodiments, the polynucleotide comprises a transgene operably linked to a ligand responsive ribozyme, wherein the ligand responsive ribozyme comprises or consists of the sequence set forth in SEQ ID NO: 2.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 3. In certain embodiments, the polynucleotide comprises a transgene operably linked to a ligand responsive ribozyme, wherein the ligand responsive ribozyme comprises or consists of the sequence set forth in SEQ ID NO: 3.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 4. In certain embodiments, the polynucleotide comprises a transgene operably linked to a ligand responsive ribozyme, wherein the ligand responsive ribozyme comprises or consists of the sequence set forth in SEQ ID NO: 4.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 5. In certain embodiments, the polynucleotide comprises a transgene operably linked to a ligand responsive ribozyme, wherein the ligand responsive ribozyme comprises or consists of the sequence set forth in SEQ ID NO: 5.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 6. In certain embodiments, the polynucleotide comprises a transgene operably linked to a ligand responsive ribozyme, wherein the ligand responsive ribozyme comprises or consists of the sequence set forth in SEQ ID NO: 6.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 7. In certain embodiments, the polynucleotide comprises a transgene operably linked to a ligand responsive ribozyme, wherein the ligand responsive ribozyme comprises or consists of the sequence set forth in SEQ ID NO: 7.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 8. In certain embodiments, the polynucleotide comprises a transgene operably linked to a ligand responsive ribozyme, wherein the ligand responsive ribozyme comprises or consists of the sequence set forth in SEQ ID NO: 8.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 9. In certain embodiments, the polynucleotide comprises a transgene operably linked to a ligand responsive ribozyme, wherein the ligand responsive ribozyme comprises or consists of the sequence set forth in SEQ ID NO: 9.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 10. In certain embodiments, the polynucleotide comprises a transgene operably linked to a ligand responsive ribozyme, wherein the ligand responsive ribozyme comprises or consists of the sequence set forth in SEQ ID NO: 10.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 11. In certain embodiments, the polynucleotide comprises a transgene operably linked to a ligand responsive ribozyme, wherein the ligand responsive ribozyme comprises or consists of the sequence set forth in SEQ ID NO: 11.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 12. In certain embodiments, the polynucleotide comprises a transgene operably linked to a ligand responsive ribozyme, wherein the ligand responsive ribozyme comprises or consists of the sequence set forth in SEQ ID NO: 12.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 13. In certain embodiments, the polynucleotide comprises a transgene operably linked to a ligand responsive ribozyme, wherein the ligand responsive ribozyme comprises or consists of the sequence set forth in SEQ ID NO: 13.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 14. In certain embodiments, the polynucleotide comprises a transgene operably linked to a ligand responsive ribozyme, wherein the ligand responsive ribozyme comprises or consists of the sequence set forth in SEQ ID NO: 14.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 15. In certain embodiments, the polynucleotide comprises a transgene operably linked to a ligand responsive ribozyme, wherein the ligand responsive ribozyme comprises or consists of the sequence set forth in SEQ ID NO: 15.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 16. In certain embodiments, the polynucleotide comprises a transgene operably linked to a ligand responsive ribozyme, wherein the ligand responsive ribozyme comprises or consists of the sequence set forth in SEQ ID NO: 16.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 17. In certain embodiments, the polynucleotide comprises a transgene operably linked to a ligand responsive ribozyme, wherein the ligand responsive ribozyme comprises or consists of the sequence set forth in SEQ ID NO: 17.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 18. In certain embodiments, the polynucleotide comprises a transgene operably linked to a ligand responsive ribozyme, wherein the ligand responsive ribozyme comprises or consists of the sequence set forth in SEQ ID NO: 18.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 19. In certain embodiments, the polynucleotide comprises a transgene operably linked to a ligand responsive ribozyme, wherein the ligand responsive ribozyme comprises or consists of the sequence set forth in SEQ ID NO: 19.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 20. In certain embodiments, the polynucleotide comprises a transgene operably linked to a ligand responsive ribozyme, wherein the ligand responsive ribozyme comprises or consists of the sequence set forth in SEQ ID NO: 20.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 21. In certain embodiments, the polynucleotide comprises a transgene operably linked to a ligand responsive ribozyme, wherein the ligand responsive ribozyme comprises or consists of the sequence set forth in SEQ ID NO: 21.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 22. In certain embodiments, the polynucleotide comprises a transgene operably linked to a ligand responsive ribozyme, wherein the ligand responsive ribozyme comprises or consists of the sequence set forth in SEQ ID NO: 22.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 23. In certain embodiments, the polynucleotide comprises a transgene operably linked to a ligand responsive ribozyme, wherein the ligand responsive ribozyme comprises or consists of the sequence set forth in SEQ ID NO: 23.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 24. In certain embodiments, the polynucleotide comprises a transgene operably linked to a ligand responsive ribozyme, wherein the ligand responsive ribozyme comprises or consists of the sequence set forth in SEQ ID NO: 24.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 25. In certain embodiments, the polynucleotide comprises a transgene operably linked to a ligand responsive ribozyme, wherein the ligand responsive ribozyme comprises or consists of the sequence set forth in SEQ ID NO: 25.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 26. In certain embodiments, the polynucleotide comprises a transgene operably linked to a ligand responsive ribozyme, wherein the ligand responsive ribozyme comprises or consists of the sequence set forth in SEQ ID NO: 26.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 27. In certain embodiments, the polynucleotide comprises a transgene operably linked to a ligand responsive ribozyme, wherein the ligand responsive ribozyme comprises or consists of the sequence set forth in SEQ ID NO: 27.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 28. In certain embodiments, the polynucleotide comprises a transgene operably linked to a ligand responsive ribozyme, wherein the ligand responsive ribozyme comprises or consists of the sequence set forth in SEQ ID NO: 28.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 29. In certain embodiments, the polynucleotide comprises a transgene operably linked to a ligand responsive ribozyme, wherein the ligand responsive ribozyme comprises or consists of the sequence set forth in SEQ ID NO: 29.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 30. In certain embodiments, the polynucleotide comprises a transgene operably linked to a ligand responsive ribozyme, wherein the ligand responsive ribozyme comprises or consists of the sequence set forth in SEQ ID NO: 30.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 31. In certain embodiments, the polynucleotide comprises a transgene operably linked to a ligand responsive ribozyme, wherein the ligand responsive ribozyme comprises or consists of the sequence set forth in SEQ ID NO: 31.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 32. In certain embodiments, the polynucleotide comprises a transgene operably linked to a ligand responsive ribozyme, wherein the ligand responsive ribozyme comprises or consists of the sequence set forth in SEQ ID NO: 32.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 33. In certain embodiments, the polynucleotide comprises a transgene operably linked to a ligand responsive ribozyme, wherein the ligand responsive ribozyme comprises or consists of the sequence set forth in SEQ ID NO: 33.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 34. In certain embodiments, the polynucleotide comprises a transgene operably linked to a ligand responsive ribozyme, wherein the ligand responsive ribozyme comprises or consists of the sequence set forth in SEQ ID NO: 34.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 35. In certain embodiments, the polynucleotide comprises a transgene operably linked to a ligand responsive ribozyme, wherein the ligand responsive ribozyme comprises or consists of the sequence set forth in SEQ ID NO: 35.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 36. In certain embodiments, the polynucleotide comprises a transgene operably linked to a ligand responsive ribozyme, wherein the ligand responsive ribozyme comprises or consists of the sequence set forth in SEQ ID NO: 36.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 37. In certain embodiments, the polynucleotide comprises a transgene operably linked to a ligand responsive ribozyme, wherein the ligand responsive ribozyme comprises or consists of the sequence set forth in SEQ ID NO: 37.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 38. In certain embodiments, the polynucleotide comprises a transgene operably linked to a ligand responsive ribozyme, wherein the ligand responsive ribozyme comprises or consists of the sequence set forth in SEQ ID NO: 38.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 39. In certain embodiments, the polynucleotide comprises a transgene operably linked to a ligand responsive ribozyme, wherein the ligand responsive ribozyme comprises or consists of the sequence set forth in SEQ ID NO: 39.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 40. In certain embodiments, the polynucleotide comprises a transgene operably linked to a ligand responsive ribozyme, wherein the ligand responsive ribozyme comprises or consists of the sequence set forth in SEQ ID NO: 40.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 41. In certain embodiments, the polynucleotide comprises a transgene operably linked to a ligand responsive ribozyme, wherein the ligand responsive ribozyme comprises or consists of the sequence set forth in SEQ ID NO: 41.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 42. In certain embodiments, the polynucleotide comprises a transgene operably linked to a ligand responsive ribozyme, wherein the ligand responsive ribozyme comprises or consists of the sequence set forth in SEQ ID NO: 42.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 43. In certain embodiments, the polynucleotide comprises a transgene operably linked to a ligand responsive ribozyme, wherein the ligand responsive ribozyme comprises or consists of the sequence set forth in SEQ ID NO: 43.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 44. In certain embodiments, the polynucleotide comprises a transgene operably linked to a ligand responsive ribozyme, wherein the ligand responsive ribozyme comprises or consists of the sequence set forth in SEQ ID NO: 44.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 45. In certain embodiments, the polynucleotide comprises a transgene operably linked to a ligand responsive ribozyme, wherein the ligand responsive ribozyme comprises or consists of the sequence set forth in SEQ ID NO: 45.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 46. In certain embodiments, the polynucleotide comprises a transgene operably linked to a ligand responsive ribozyme, wherein the ligand responsive ribozyme comprises or consists of the sequence set forth in SEQ ID NO: 46.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 47. In certain embodiments, the polynucleotide comprises a transgene operably linked to a ligand responsive ribozyme, wherein the ligand responsive ribozyme comprises or consists of the sequence set forth in SEQ ID NO: 47.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 48. In certain embodiments, the polynucleotide comprises a transgene operably linked to a ligand responsive ribozyme, wherein the ligand responsive ribozyme comprises or consists of the sequence set forth in SEQ ID NO: 48.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 49. In certain embodiments, the polynucleotide comprises a transgene operably linked to a ligand responsive ribozyme, wherein the ligand responsive ribozyme comprises or consists of the sequence set forth in SEQ ID NO: 49.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in SEQ ID NO: 50. In certain embodiments, the polynucleotide comprises a transgene operably linked to a ligand responsive ribozyme, wherein the ligand responsive ribozyme comprises or consists of the sequence set forth in SEQ ID NO: 50.

In certain embodiments, the polynucleotide comprises a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to the sequence set forth in any one of SEQ ID NOs: 51-476. In certain embodiments, the polynucleotide comprises a transgene operably linked to a ligand responsive ribozyme, wherein the ligand responsive ribozyme comprises or consists of the sequence set forth in any one of SEQ ID NOs: 51-476.

In certain embodiments, the polynucleotide comprises a transgene operably linked to a ligand responsive ribozyme, wherein the ligand responsive ribozyme comprises a nucleic acid sequence having at least 85% sequence identity to the sequence set forth in any one of SEQ ID NOs: 1-476. In certain embodiments, the polynucleotide comprises a transgene operably linked to a ligand responsive ribozyme, wherein the ligand responsive ribozyme comprises a nucleic acid sequence having at least 86% sequence identity to the sequence set forth in any one of SEQ ID NOs: 1-476. In certain embodiments, the polynucleotide comprises a transgene operably linked to a ligand responsive ribozyme, wherein the ligand responsive ribozyme comprises a nucleic acid sequence having at least 87% sequence identity to the sequence set forth in any one of SEQ ID NOs: 1-476. In certain embodiments, the polynucleotide comprises a transgene operably linked to a ligand responsive ribozyme, wherein the ligand responsive ribozyme comprises a nucleic acid sequence having at least 88% sequence identity to the sequence set forth in any one of SEQ ID NOs: 1-476. In certain embodiments, the polynucleotide comprises a transgene operably linked to a ligand responsive ribozyme, wherein the ligand responsive ribozyme comprises a nucleic acid sequence having at least 89% sequence identity to the sequence set forth in any one of SEQ ID NOs: 1-476. In certain embodiments, the polynucleotide comprises a transgene operably linked to a ligand responsive ribozyme, wherein the ligand responsive ribozyme comprises a nucleic acid sequence having at least 90% sequence identity to the sequence set forth in any one of SEQ ID NOs: 1-476. In certain embodiments, the polynucleotide comprises a transgene operably linked to a ligand responsive ribozyme, wherein the ligand responsive ribozyme comprises a nucleic acid sequence having at least 91% sequence identity to the sequence set forth in any one of SEQ ID NOs: 1-476. In certain embodiments, the polynucleotide comprises a transgene operably linked to a ligand responsive ribozyme, wherein the ligand responsive ribozyme comprises a nucleic acid sequence having at least 92% sequence identity to the sequence set forth in any one of SEQ ID NOs: 1-476. In certain embodiments, the polynucleotide comprises a transgene operably linked to a ligand responsive ribozyme, wherein the ligand responsive ribozyme comprises a nucleic acid sequence having at least 93% sequence identity to the sequence set forth in any one of SEQ ID NOs: 1-476. In certain embodiments, the polynucleotide comprises a transgene operably linked to a ligand responsive ribozyme, wherein the ligand responsive ribozyme comprises a nucleic acid sequence having at least 94% sequence identity to the sequence set forth in any one of SEQ ID NOs: 1-476. In certain embodiments, the polynucleotide comprises a transgene operably linked to a ligand responsive ribozyme, wherein the ligand responsive ribozyme comprises a nucleic acid sequence having at least 95% sequence identity to the sequence set forth in any one of SEQ ID NOs: 1-476. In certain embodiments, the polynucleotide comprises a transgene operably linked to a ligand responsive ribozyme, wherein the ligand responsive ribozyme comprises a nucleic acid sequence having at least 96% sequence identity to the sequence set forth in any one of SEQ ID NOs: 1-476. In certain embodiments, the polynucleotide comprises a transgene operably linked to a ligand responsive ribozyme, wherein the ligand responsive ribozyme comprises a nucleic acid sequence having at least 97% sequence identity to the sequence set forth in any one of SEQ ID NOs: 1-476. In certain embodiments, the polynucleotide comprises a transgene operably linked to a ligand responsive ribozyme, wherein the ligand responsive ribozyme comprises a nucleic acid sequence having at least 98% sequence identity to the sequence set forth in any one of SEQ ID NOs: 1-476. In certain embodiments, the polynucleotide comprises a transgene operably linked to a ligand responsive ribozyme, wherein the ligand responsive ribozyme comprises a nucleic acid sequence having at least 99% sequence identity to the sequence set forth in any one of SEQ ID NOs: 1-476. In certain embodiments, the polynucleotide comprises a transgene operably linked to a ligand responsive ribozyme, wherein the ligand responsive ribozyme comprises a nucleic acid sequence set forth in any one of SEQ ID NOs: 1-476. In certain embodiments, the polynucleotide comprises a transgene operably linked to a ligand responsive ribozyme, wherein the ligand responsive ribozyme consists of a nucleic acid sequence set forth in any one of SEQ ID NOs: 1-476.

In certain embodiments, the polynucleotide further comprises a transcription terminator (e.g., a polyadenylation sequence). In certain embodiments, the transcription terminator is 3′ to the transgene. The transcription terminator may be any sequence that effectively terminates transcription, and a skilled artisan would appreciate that such sequences can be isolated from any genes that are expressed in the cell in which transcription of the transgene is desired. In certain embodiments, the transcription terminator comprises a polyadenylation sequence. In certain embodiments, the polyadenylation sequence is identical or substantially identical to the endogenous polyadenylation sequence of the transgene. In certain embodiments, the polyadenylation sequence is an exogenous polyadenylation sequence. In addition to possessing transcription termination functions, the polyadenylation sequence is also known in the art to protect the transcript on which it resides from enzymatic degradation in the cytoplasm.

In certain embodiments, the ligand responsive ribozyme is inserted into a 3′ untranslated region and/or the 5′ UTR of the transgene. In certain embodiments, the ligand responsive ribozyme is inserted into a 3′ UTR of the transgene. In certain embodiments, the ligand responsive ribozyme is inserted into a 5′ UTR of the transgene.

In certain embodiments, the ligand responsive ribozyme is inserted into a 3′ UTR of the transgene and upstream of a polyadenylation sequence. In the absence of ligand (e.g., theophylline or derivative thereof), the ligand responsive ribozyme may inhibit transgene expression by facilitating self-cleavage, leading to the cleaving of the polyadenylation sequence off of the transcript, resulting in transcript instability and degradation. In the presence of ligand (e.g., theophylline or derivative thereof), the ligand-bound ligand responsive ribozyme is in a conformation in which self-cleavage cannot occur, thus resulting in the transcript having an intact polyadenylation sequence, allowing for transgene expression to occur.

In certain embodiments, the transgene encodes a polypeptide. In certain embodiments, the transgene encodes a therapeutic protein. In certain embodiments, the transgene encodes an antibody or a fragment thereof (e.g., a Fab, scFv, or full-length antibody). In certain embodiments, the transgene encodes an scFv, nanobody, or VHH. In certain embodiments, the transgene encodes a non-coding RNA. In certain embodiments the transgene comprises one or more sequences encoding an RNA molecule. Suitable RNA molecules include, without limitation, miRNA, shRNA, siRNA, antisense RNA, gRNA, antagomirs, miRNA sponges, RNA aptazymes, RNA aptamers, mRNA, lncRNAs, ribozymes, and synthetic RNAs known in the art.

In certain embodiments, the transgene encodes one or more polypeptides, or a fragment thereof. Such transgenes can comprise the complete coding sequence of a polypeptide, or only a fragment of a coding sequence of a polypeptide. In certain embodiments, the transgene encodes a polypeptide that is useful to treat a disease or disorder in a subject. Suitable polypeptides include, without limitation, β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF, acidic FGF, and FGF21), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β) and soluble fragments thereof, and the like; soluble receptors, such as soluble TGFβ receptors (e.g., soluble TGFβR2 receptors (sTGFβR2)), soluble TNF-α receptors, soluble interleukin receptors (e.g., soluble IL-1 receptors and soluble type II IL-1 receptors), soluble γ/Δ T cell receptors, ligand-binding fragments of a soluble receptor, and the like; enzymes, such as a-glucosidase, imiglucerase, β-glucocerebrosidase, and alglucerase; enzyme activators, such as tissue plasminogen activator; chemokines, such as IP-10, monokine induced by interferon-gamma (Mig), Groα/IL-8, RANTES, MIP-1a, MIP-1β, MCP-1, PF-4, and the like; angiogenic agents, such as vascular endothelial growth factors (VEGFs, e.g., VEGF121, VEGF165, VEGF-C, VEGF-2), glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastrin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); tissue factors; macrophage activating factors; tumor necrosis factor (TNF); neutrophil chemotactic factor (NCF); tissue inhibitors of metalloproteinases; vasoactive intestinal peptide; angiogenin; angiotrofin; fibrin; hirudin; IL-1 receptor antagonists; ciliary neurotrophic factor (CNTF); brain-derived neurotrophic factor (BDNF); neurotrophins 3 and 4/5 (NT-3 and -4/5); glial cell derived neurotrophic factor (GDNF); aromatic amino acid decarboxylase (AADC); Factor VIII, Factor IX, Factor X; dystrophin or mini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase, glucose transporter, aldolase A, β-enolase, glycogen synthase; lysosomal enzymes, such as iduronate-2-sulfatase (12S), and arylsulfatase A; and mitochondrial proteins, such as frataxin.

In certain embodiments, the transgene encodes an immune cell receptor. Suitable immune cell receptors include T cell receptors (TCRs) and chimeric antigen receptors (CARs).

In certain embodiments, the transgene encodes a protein that may be defective in one or more lysosomal storage diseases. Suitable proteins include, without limitation, α-sialidase, cathepsin A, α-mannosidase, β-mannosidase, glycosylasparaginase, α-fucosidase, α-N-acetylglucosaminidase, β-galactosidase, β-hexosaminidase α-subunit, β-hexosaminidase β-subunit, GM2 activator protein, glucocerebrosidase, Saposin C, Arylsulfatase A, Saposin B, formyl-glycine generating enzyme, β-galactosylceramidase, α-galactosidase A, iduronate sulfatase, α-iduronidase, heparan N-sulfatase, acetyl-CoA transferase, N-acetyl glucosaminidase, β-glucuronidase, N-acetyl glucosamine 6-sulfatase, N-acetylgalactosamine 4-sulfatase, galactose 6-sulfatase, hyaluronidase, a-glucosidase, acid sphingomyelinase, acid ceramidase, acid lipase, capthepsin K, tripeptidyl peptidase, palmitoyl-protein thioesterase, cystinosin, sialin, UDP-N-acetylglucosamine, phosphotransferase γ-subunit, mucolipin-1, LAMP-2, NPC1, CLN3, CLN 6, CLN 8, LYST, MYOV, RAB27A, mclanophilin, and AP3 β-subunit.

In certain embodiments, the transgene encodes an antibody or a fragment thereof (e.g., a Fab, scFv, or full-length antibody). Suitable antibodies include, without limitation, muromonab-cd3, efalizumab, tositumomab, daclizumab, nebacumab, catumaxomab, edrecolomab, abciximab, rituximab, basiliximab, palivizumab, infliximab, trastuzumab, adalimumab, ibritumomab tiuxetan, omalizumab, cetuximab, bevacizumab, natalizumab, panitumumab, ranibizumab, eculizumab, certolizumab, ustekinumab, canakinumab, golimumab, ofatumumab, tocilizumab, denosumab, belimumab, ipilimumab, brentuximab vedotin, pertuzumab, raxibacumab, obinutuzumab, alemtuzumab, siltuximab, ramucirumab, vedolizumab, blinatumomab, nivolumab, pembrolizumab, idarucizumab, necitumumab, dinutuximab, secukinumab, mepolizumab, alirocumab, evolocumab, daratumumab, elotuzumab, ixekizumab, reslizumab, olaratumab, bezlotoxumab, atezolizumab, obiltoxaximab, inotuzumab ozogamicin, brodalumab, guselkumab, dupilumab, sarilumab, avelumab, ocrelizumab, emicizumab, benralizumab, gemtuzumab ozogamicin, durvalumab, burosumab, erenumab, galcanezumab, lanadelumab, mogamulizumab, tildrakizumab, cemiplimab, fremanezumab, ravulizumab, emapalumab, ibalizumab, moxetumomab, caplacizumab, romosozumab, risankizumab, polatuzumab, eptinezumab, leronlimab, sacituzumab, brolucizumab, isatuximab, and teprotumumab.

In certain embodiments, the transgene encodes one or more transcription factors.

In certain embodiments, the transgene encodes a nuclease. Suitable nucleases include, without limitation, zinc fingers nucleases (ZFN) (see, e.g., Porteus, and Baltimore (2003) Science 300:763; Miller et al. (2007) Nat. Biotechnol. 25:778-785; Sander et al. (2011) Nature Methods 8:67-69; and Wood et al. (2011) Science 333:307, each of which is hereby incorporated by reference in its entirety); transcription activator-like effectors nucleases (TALEN) (see, e.g., Wood et al. (2011) Science 333:307; Boch et al. (2009) Science 326:1509-1512; Moscou and Bogdanove (2009) Science 326; 1501; Christian et al. (2010) Genetics 186:757-761; Miller et al. (2011) Nat. Biotechnol. 29:143-148; Zhang et al. (2011) Nat. Biotechnol. 29:149-153; and Reyon et al. (2012) Nat. Biotechnol. 30 (5): 460-465, each of which is hereby incorporated by reference in its entirety); homing endonucleases; meganucleases (see, e.g., U.S. Patent Publication No. US 2014/0121115, which is hereby incorporated by reference in its entirety); and RNA-guided nucleases (see, e.g., Makarova et al. (2018) The CRISPR Journal 1 (5): 325-336; and Adli (2018) Nat. Communications 9:1911, each of which is hereby incorporated by reference in its entirety).

In certain embodiments, the transgene encodes an RNA-guided nuclease. Suitable RNA-guided nucleases include, without limitation, Class I and Class II clustered regularly interspaced short palindromic repeats (CRISPR)-associated nucleases. Class I is divided into types I, III, and IV, and includes, without limitation, type I (Cas3), type I-A (Cas8a, Cas5), type I-B (Cas8b), type I-C(Cas8c), type 1-D (Cas10d), type I-E (Cse1, Cse2), type I-F (Csy1, Csy2, Csy3), type I-U (GSU0054), type III (Cas10), type III-A (Csm2), type III-B (Cmr5), type III-C(Csx10 or Csx11), type III-D (Csx10), and type IV (Csf1). Class II is divided into types II, V, and VI, and includes, without limitation, type II (Cas9), type II-A (Csn2), type II-B (Cas4), type V (Cpf1, C2c1, C2c3), and type VI (Cas13a, Cas13b, Cas13c). RNA-guided nucleases also include naturally-occurring Class II CRISPR nucleases such as Cas9 (Type II) or Cas12a/Cpf1 (Type V), as well as other nucleases derived or obtained therefrom. Exemplary Cas9 nucleases that may be used in the present invention include, but are not limited to, S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9).

In certain embodiments, the transgene comprises a suicide gene. Cells that express a suicide gene are conferred sensitivity to an agent, e.g., a drug, such that when the cell expressing the suicide gene is contacted with or exposed to the agent, it causes the cell to die. Various suicide genes are known to those of skill in the art, for example, without limitation, Herpes Simplex Virus Thymidine Kinase (HSV-TK), for which the agent is ganciclovir, and inducible Caspase 9 (iCasp9), for which the agent is the small molecule AP20187. Other examples of suicide genes include, without limitation, sequences comprising a minimal epitope based on an epitope of CD20 that is recognized by a therapeutic anti-CD20 antibody (e.g., rituximab), described in e.g., PCT Publication No. WO2013153391A1, which is herein incorporated by reference in its entirety; and a truncated epidermal growth factor receptor (EGFR) that is recognized by a therapeutic anti-EGFR antibody (e.g., cetuximab), described in e.g., PCT Publication No. WO2011056894A2, which is herein incorporated by reference in its entirety.

In certain embodiments, the transgene encodes reporter sequences, which upon expression produce a detectable signal. Such reporter sequences include, without limitation, DNA sequences encoding β-lactamase, β-galactosidase (LacZ), alkaline phosphatase, thymidine kinase, green fluorescent protein (GFP), red fluorescent protein (RFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

In certain embodiments, the transgene is operably linked to a transcriptional regulatory element (TRE), to control expression of an RNA or polypeptide encoded by the transgene. In certain embodiments, the TRE comprises a constitutive promoter. In certain embodiments, the TRE can be active in any mammalian cell (e.g., any human cell). In certain embodiments, the TRE is active in a broad range of human cells. Such TREs may comprise constitutive promoter and/or enhancer elements including any of those described herein, and any of those known to one of skill in the art. In certain embodiments, the TRE comprises an inducible promoter. In certain embodiments, the TRE may be a tissue-specific TRE, i.e., it is active in specific tissue(s) and/or organ(s). A tissue-specific TRE comprises one or more tissue-specific promoter and/or enhancer elements, and optionally one or more constitutive promoter and/or enhancer elements. A skilled artisan would appreciate that tissue-specific promoter and/or enhancer elements can be isolated from genes specifically expressed in the tissue by methods well known in the art.

Suitable promoters include, e.g., cytomegalovirus promoter (CMV) (Stinski et al. (1985) Journal of Virology 55 (2): 431-441); CMV early enhancer/chicken β-actin (CBA) promoter/rabbit β-globin intron (CAG) (Miyazaki et al. (1989) Gene 79 (2): 269-277); CB^{SB} (Jacobson et al. (2006) Molecular Therapy 13 (6): 1074-1084); human elongation factor 1α promoter (EF1α) (Kim et al. (1990) Gene 91 (2): 217-223); human phosphoglycerate kinase promoter (PGK) (Singer-Sam et al. (1984) Gene 32 (3): 409-417); mitochondrial heavy-strand promoter (Lodeiro et al. (2012) PNAS 109 (17): 6513-6518); and ubiquitin promoter (Wulff et al. (1990) FEBS Letters 261:101-105). In certain embodiments, the TRE comprises a cytomegalovirus (CMV) promoter/enhancer, an SV40 promoter, a chicken beta actin (CBA) promoter, an smCBA promoter, a human elongation factor 1 alpha (EF1α) promoter, a minute virus of mouse (MVM) intron which comprises transcription factor binding sites, a human phosphoglycerate kinase (PGK1) promoter, a human ubiquitin C (Ubc) promoter, a human beta actin promoter, a human neuron-specific enolase (ENO2) promoter, a human beta-glucuronidase (GUSB) promoter, a rabbit beta-globin element, a human calmodulin 1 (CALM1) promoter, a human ApoE/C-I hepatic control region (HCR1), a human al-antitrypsin (hAAT) promoter, an extended HCR1, a HS-CRM8 element of an hAAT promoter, a human transthyretin (TTR) promoter, and/or a human Methyl-CpG Binding Protein 2 (MeCP2) promoter. Any of the TREs described herein can be combined in any order to drive efficient transcription.

In certain embodiments, the TRE is liver-specific. Exemplary liver-specific TREs may comprise one or more elements from, without limitation, the ApoA-I promoter, the ApoA-II promoter, the ApoA-IV promoter, the ApoB promoter, the ApoC-I promoter, the ApoC-II promoter, the ApoC-III promoter, the ApoE promoter, the albumin promoter, the α-fetoprotein promoter, the phosphoenolpyruvate carboxykinase 1 (PCK1) promoter, the phosphoenolpyruvate carboxykinase 2 (PCK2) promoter, the transthyretin (TTR) promoter, the α-antitrypsin (AAT or SERPINA1) promoter, the TK (thymidine kinase) promoter, the hemopexin promoter, the alcohol dehydrogenase 6 promoter, the cholesterol 7alpha-hydroxylase promoter, the factor IX promoter, the α-microglobulin promoter, the SV40 promoter, the CMV promoter, the Rous Sarcoma Virus-LTR promoter and the HBV promoter.

In certain embodiments, the TRE is muscle specific. Exemplary muscle-specific TREs may comprise one or more elements from, without limitation, the human skeletal muscle α-actin (HSA) promoter, the muscle creatine kinase (MCK) promoter, the MHCK7 promoter, the dMCK promoter, the tMCK promoter, the CK6 promoter, the CK8 promoter, the CK8e promoter, the human desmin (DES) promoter or variant thereof, the cardiac troponin T (cTnT) promoter, the myosin light-chain (MLC2v) promoter, the human α-myosin heavy chain gene (αMHC) promoter, the MLC promoter, the human troponin I (TNNI1) promoter, the ΔUSEx3 promoter, the SPcΔ5-12 promoter, the SP-301 promoter, the MH promoter, and the Sk-CRM4/DES promoter.

In certain embodiments, the native promoter for the transgene may be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In certain embodiments, the polynucleotide is optimized, e.g., by codon/RNA optimization, replacement with heterologous signal sequences, and/or elimination of mRNA instability elements. Methods to generate optimized polynucleotides for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, all of which are herein incorporated by reference in their entireties. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In certain embodiments, it can be desirable to alter one or more codons to encode a conservative mutation, e.g., a similar amino acid with similar chemical structure and properties and/or function as the original amino acid. Such methods can increase expression of the encoded capsid protein relative to the expression of the capsid encoded by polynucleotides that have not been optimized.

In another aspect, the present disclosure provides a vector comprising a polynucleotide described herein. Suitable vectors, include, without limitation, plasmids, viruses, cosmids, artificial chromosomes, linear DNA, and mRNA. In certain embodiments, the vector is a plasmid or a viral vector. In certain embodiments, the vector is a retrovirus vector, a herpes virus vector, a baculovirus vector, or an adenovirus vector. In certain embodiments, the vector is an expression vector.

In certain embodiments, the polynucleotide (e.g., a ligand responsive ribozyme optionally operably linked to a transgene) is integrated into the genome of a target cell (e.g., immune cell or precursor thereof, e.g., T cell, NK cell, NKT cell, or precursor thereof). In certain embodiments, the polynucleotide is integrated via random integration, a site-specific integration, or a biased integration. In certain embodiments, the site-specific integration can be non-assisted or assisted. In certain embodiments, in assisted site-specific integration, the polynucleotide is co-delivered with a site-directed nuclease. In certain embodiments, the site-directed nuclease comprises the polynucleotide with 5′ and 3′ nucleotide sequence extensions that contain a percentage homology to upstream and downstream regions of the site of genomic integration. In certain embodiments, the polynucleotide with homologous nucleotide extensions enables genomic integration by homologous recombination, microhomology-mediated end joining, or nonhomologous end-joining.

In certain embodiments, the site-specific integration occurs at a safe harbor site. Genomic safe harbor sites are able to accommodate the integration of new genetic material in a manner that ensures that the newly inserted genetic elements function reliably (for example, are expressed at a therapeutically effective level) and do not cause deleterious alterations to the host genome that cause a risk to the host organism. Potential genomic safe harbors include, without limitation, intronic sequences of the human albumin gene, the adeno-associated virus site 1 (AAVS1), a naturally occurring site of integration of AAV virus on chromosome 19, the site of the chemokine (C-C motif) receptor 5 (CCR5) gene, and the site of the human ortholog of the mouse Rosa26 locus.

In certain embodiments, the site-specific integration occurs at a site that disrupts expression of a target gene. In certain embodiments, disruption of target gene expression occurs by site-specific integration at introns, exons, promoters, genetic elements, enhancers, suppressors, start codons, stop codons, and response elements.

In certain embodiments, the site-specific integration occurs at a site that results in enhanced expression of a target gene. In certain embodiments, enhancement of target gene expression occurs by site-specific integration at introns, exons, promoters, genetic elements, enhancers, suppressors, start codons, stop codons, and response elements.

In certain embodiments, enzymes may be used to create strand breaks in the host genome to facilitate delivery or integration of the polynucleotide. In certain embodiments, enzymes create single-strand breaks. In certain embodiments, enzymes create double-strand breaks. In certain embodiments, examples of break-inducing enzymes include, but are not limited to, transposases, integrases, endonucleases, CRISPR-Cas9, transcription activator-like effector nucleases (TALENs), zinc finger nucleases (ZFNs), Cas-CLOVER™, and CPF1. In certain embodiments, break-inducing enzymes can be delivered to the cell encoded in DNA, encoded in mRNA, as a protein, as a nucleoprotein complex with a guide RNA (gRNA).

Vectors (e.g., expression vectors) can be introduced into cells (using any techniques known in the art) for propagation of the vector and/or for expression of a polypeptide encoded by the vector. Accordingly, in another aspect, the instant disclosure provides a recombinant cell comprising a polynucleotide or a vector (e.g., an expression vector) described herein.

Suitable vectors, include, without limitation, plasmids, minimal vectors (e.g., minicircles, Nanoplasmids™, doggybones, MIDGE vectors, and the like), viruses, cosmids, artificial chromosomes, linear DNA, and mRNA. In certain embodiments, the first nucleic acid vector and/or the second nucleic acid vector is a DNA plasmid or a DNA minimal vector. Any DNA plasmid or DNA minimal vector that can accommodate the necessary vector elements can be used for the first nucleic acid vector and the second nucleic acid vector. Suitable DNA minimal vectors include, without limitation, linear covalently closed DNA (e.g., ministring DNA), linear covalently closed dumbbell shaped DNA (e.g., doggybone DNA, dumbbell DNA), minicircles, Nanoplasmids™, minimalistic immunologically defined gene expression (MIDGE) vectors, and others known to those of skill in the art. DNA minimal vectors and their methods of production are described in, e.g., U.S. patents application Nos. 20100233814, 20120282283, 20130216562, 20150218565, 20150218586, 20160008488, 20160215296, 20160355827, 20190185924, 20200277624, and 20210010021, all of which are herein incorporated by reference in their entireties.

A variety of host cells and expression vector systems can be utilized. These include but are not limited to microorganisms such as bacteria (e.g., E. coli and B. subtilis) transformed with, e.g., recombinant bacteriophage DNA, plasmid DNA or cosmid DNA expression vectors containing capsid protein coding sequences; yeast (e.g., Saccharomyces Pichia) transformed with, e.g., recombinant yeast expression vectors containing capsid protein coding sequences; insect cell systems infected with, e.g., recombinant virus expression vectors (e.g., baculovirus) containing capsid protein coding sequences; plant cell systems (e.g., green algae such as Chlamydomonas reinhardtii) infected with, e.g., recombinant virus expression vectors (e.g., cauliflower mosaic virus, CaMV; tobacco mosaic virus, TMV) or transformed with, e.g., recombinant plasmid expression vectors (e.g., Ti plasmid) containing capsid protein coding sequences; or mammalian cell systems (e.g., COS (e.g., COSI or COS), CHO, BHK, MDCK, HEK 293, NS0, PER.C6, VERO, CRL7O3O, HsS78Bst, HeLa, and NIH 3T3, HEK-293T, HepG2, SP210, R1.1, B-W, L-M, BSC1, BSC40, YB/20 and BMT10 cells) harboring, e.g., recombinant expression constructs containing promoters derived from the genome of mammalian cells (e.g., metallothionein promoter) or from mammalian viruses (e.g., the adenovirus late promoter; the vaccinia virus 7.5K promoter). In certain embodiments, suitable cells are human cells, e.g., human cell lines. In certain embodiments, a mammalian expression vector is pOptiVEC™ or pcDNA3.3. In certain embodiments, bacterial cells such as Escherichia coli, or eukaryotic cells (e.g., mammalian cells), are suitable. For example, mammalian cells such as CHO or HEK293 cells, together with a vector containing the major intermediate early gene promoter element from human cytomegalovirus is an effective expression system that can be used in conjunction with the polynucleotides described herein.

In bacterial systems, a number of expression vectors can be advantageously selected depending upon the use intended. For example, when a large quantity of polypeptide is to be produced, vectors which direct the expression of high levels of fusion protein products that are readily purified can be desirable. Such vectors include, but are not limited to, the E. coli expression vector pUR278 (Ruether U & Mueller-Hill B (1983) EMBO J 2:1791-1794); pIN vectors (Inouye S & Inouye M (1985) Nuc Acids Res 13:3101-3109; Van Heeke G & Schuster S M (1989) J Biol Chem 24:5503-5509); and the like, all of which are herein incorporated by reference in their entireties. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV), for example, can be used as a vector to express foreign genes. The virus grows in Spodoptera frugiperda cells. The transgene sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the transgene sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the capsid protein molecule in infected hosts (see, e.g., Logan J & Shenk T (1984) PNAS 81 (12): 3655-9, which is herein incorporated by reference in its entirety). Specific initiation signals can also be required for efficient translation of inserted capsid protein coding sequences. These signals include the ATG initiation codon and adjacent sequences. Furthermore, the initiation codon must be in phase with the reading frame of the desired transgene sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al. (1987) Methods Enzymol. 153:516-544, which is herein incorporated by reference in its entirety).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NS0 (a murine myeloma cell line that does not endogenously produce any immunoglobulin chains), CRL7O3O, COS (e.g., COSI or COS), PER.C6, VERO, HsS78Bst, HEK-293T, HepG2, SP210, R1.1, B-W, L-M, BSC1, BSC40, YB/20, BMT10 and HsS78Bst cells.

For long-term, high-yield production of recombinant proteins, stable expression cells can be generated. For example, cell lines which stably express a capsid protein described herein can be engineered.

In certain embodiments, rather than using expression vectors which contain viral origins of replication, host cells can be transformed with a polynucleotide (e.g., DNA or RNA) controlled by appropriate transcriptional regulatory elements (e.g., promoter, enhancer, sequences, transcription terminators, polyadenylation sites, etc.), and a selectable marker. Following the introduction of polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express a capsid protein described herein or a fragment thereof.

A number of selection systems can be used, including but not limited to the herpes simplex virus thymidine kinase (Wigler M et al. (1977) Cell 11 (1): 223-32); hypoxanthineguanine phosphoribosyltransferase (Szybalska E H & Szybalski W (1962) PNAS 48 (12): 2026-2034); and adenine phosphoribosyltransferase (Lowy I et al. (1980) Cell 22 (3): 817-23) genes in tk-, hgprt- or aprt-cells, respectively, all of which are herein incorporated by reference in their entireties. Also, antimetabolite resistance can be used as the basis of selection for the following genes: dhfr, which confers resistance to methotrexate (Wigler M et al. (1980) PNAS 77 (6): 3567-70; O'Hare K et al. (1981) PNAS 78:1527-31); gpt, which confers resistance to mycophenolic acid (Mulligan R C & Berg P (1981) PNAS 78 (4): 2072-6); neo, which confers resistance to the aminoglycoside G-418 (Wu G Y & Wu C H (1991) Biotherapy 3:87-95; Tolstoshev P (1993) Ann Rev Pharmacol Toxicol 32:573-596; Mulligan R C (1993) Science 260:926-932; and Morgan R A & Anderson W F (1993) Ann Rev Biochem 62:191-217; Nabel G J & Felgner P L (1993) Trends Biotechnol 11(5): 211-5); and hygro, which confers resistance to hygromycin (Santerre R F et al. (1984) Gene 30(1-3): 147-56), all of which are herein incorporated by reference in their entireties. Methods commonly known in the art of recombinant DNA technology can be routinely applied to select the desired recombinant clone and such methods are described, for example, in Ausubel F M et al. (eds.), Current Protocols in Molecular Biology, John Wiley & Sons, NY (1993); Kriegler M, Gene Transfer and Expression, A Laboratory Manual, Stockton Press, NY (1990); and in Chapters 12 and 13, Dracopoli N C et al. (eds.), Current Protocols in Human Genetics, John Wiley & Sons, NY (1994); Colbère-Garapin F et al. (1981) J Mol Biol 150:1-14, all of which are herein incorporated by reference in their entireties.

### III. Recombinant Adeno-Associated Virus (rAAV) Genomes and Compositions

In another aspect, the present disclosure provides recombinant adeno-associated virus (rAAV) genomes comprising a polynucleotide comprising a transgene operably linked to a ligand responsive ribozyme described herein.

In certain embodiments, the rAAV genome comprises a polynucleotide comprising a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to any one of the sequences set forth in SEQ ID NOs: 1-50. In certain embodiments, the rAAV genome comprises a polynucleotide comprising a transgene operably linked to a ligand responsive ribozyme, wherein the ligand responsive ribozyme comprises or consisting of any one of the sequences set forth in SEQ ID NOs: 1-50.

In certain embodiments, the rAAV genome comprises a polynucleotide comprising a transgene operably linked to a ligand responsive ribozyme, wherein the ligand responsive ribozyme comprises a nucleic acid sequence having at least 85%, at least 86%, at least 87%, at least 88%, at least 89%, at least 90%, at least 91, at least 92%, at least 93%, at least 94%, at least 95%, at least 96%, at least 97%, at least 98%, or at least 99% sequence identity to any one of the sequences set forth in any one of SEQ ID NOs: 51-476. In certain embodiments, the rAAV genome comprises a polynucleotide comprising a transgene operably linked to a ligand responsive ribozyme, wherein the ligand responsive ribozyme comprises or consisting of any one of the sequences set forth in any one of SEQ ID NOs: 51-476.

In certain embodiments, the rAAV genome comprises a polynucleotide comprising a transgene operably linked to a ligand responsive ribozyme, wherein the ligand responsive ribozyme comprises a nucleic acid sequence having at least 85% sequence identity to any one of the sequences set forth in any one of SEQ ID NOs: 1-476. In certain embodiments, the rAAV genome comprises a polynucleotide comprising a transgene operably linked to a ligand responsive ribozyme, wherein the ligand responsive ribozyme comprises a nucleic acid sequence having at least 86% sequence identity to any one of the sequences set forth in any one of SEQ ID NOs: 1-476. In certain embodiments, the rAAV genome comprises a polynucleotide comprising a transgene operably linked to a ligand responsive ribozyme, wherein the ligand responsive ribozyme comprises a nucleic acid sequence having at least 87% sequence identity to any one of the sequences set forth in any one of SEQ ID NOs: 1-476. In certain embodiments, the rAAV genome comprises a polynucleotide comprising a transgene operably linked to a ligand responsive ribozyme, wherein the ligand responsive ribozyme comprises a nucleic acid sequence having at least 88% sequence identity to any one of the sequences set forth in any one of SEQ ID NOs: 1-476. In certain embodiments, the rAAV genome comprises a polynucleotide comprising a transgene operably linked to a ligand responsive ribozyme, wherein the ligand responsive ribozyme comprises a nucleic acid sequence having at least 89% sequence identity to any one of the sequences set forth in any one of SEQ ID NOs: 1-476. In certain embodiments, the rAAV genome comprises a polynucleotide comprising a transgene operably linked to a ligand responsive ribozyme, wherein the ligand responsive ribozyme comprises a nucleic acid sequence having at least 90% sequence identity to any one of the sequences set forth in any one of SEQ ID NOs: 1-476. In certain embodiments, the rAAV genome comprises a polynucleotide comprising a transgene operably linked to a ligand responsive ribozyme, wherein the ligand responsive ribozyme comprises a nucleic acid sequence having at least 91% sequence identity to any one of the sequences set forth in any one of SEQ ID NOs: 1-476. In certain embodiments, the rAAV genome comprises a polynucleotide comprising a transgene operably linked to a ligand responsive ribozyme, wherein the ligand responsive ribozyme comprises a nucleic acid sequence having at least 92% sequence identity to any one of the sequences set forth in any one of SEQ ID NOs: 1-476. In certain embodiments, the rAAV genome comprises a polynucleotide comprising a transgene operably linked to a ligand responsive ribozyme, wherein the ligand responsive ribozyme comprises a nucleic acid sequence having at least 93% sequence identity to any one of the sequences set forth in any one of SEQ ID NOs: 1-476. In certain embodiments, the rAAV genome comprises a polynucleotide comprising a transgene operably linked to a ligand responsive ribozyme, wherein the ligand responsive ribozyme comprises a nucleic acid sequence having at least 94% sequence identity to any one of the sequences set forth in any one of SEQ ID NOs: 1-476. In certain embodiments, the rAAV genome comprises a polynucleotide comprising a transgene operably linked to a ligand responsive ribozyme, wherein the ligand responsive ribozyme comprises a nucleic acid sequence having at least 95% sequence identity to any one of the sequences set forth in any one of SEQ ID NOs: 1-476. In certain embodiments, the rAAV genome comprises a polynucleotide comprising a transgene operably linked to a ligand responsive ribozyme, wherein the ligand responsive ribozyme comprises a nucleic acid sequence having at least 96% sequence identity to any one of the sequences set forth in any one of SEQ ID NOs: 1-476. In certain embodiments, the rAAV genome comprises a polynucleotide comprising a transgene operably linked to a ligand responsive ribozyme, wherein the ligand responsive ribozyme comprises a nucleic acid sequence having at least 97% sequence identity to any one of the sequences set forth in any one of SEQ ID NOs: 1-476. In certain embodiments, the rAAV genome comprises a polynucleotide comprising a transgene operably linked to a ligand responsive ribozyme, wherein the ligand responsive ribozyme comprises a nucleic acid sequence having at least 98% sequence identity to any one of the sequences set forth in any one of SEQ ID NOs: 1-476. In certain embodiments, the rAAV genome comprises a polynucleotide comprising a transgene operably linked to a ligand responsive ribozyme, wherein the ligand responsive ribozyme comprises a nucleic acid sequence having at least 99% sequence identity to any one of the sequences set forth in any one of SEQ ID NOs: 1-476. In certain embodiments, the rAAV genome comprises a polynucleotide comprising a transgene operably linked to a ligand responsive ribozyme, wherein the ligand responsive ribozyme comprises a nucleic acid sequence set forth in any one of SEQ ID NOs: 1-476. In certain embodiments, the rAAV genome comprises a polynucleotide comprising a transgene operably linked to a ligand responsive ribozyme, wherein the ligand responsive ribozyme consists of a nucleic acid sequence set forth in any one of SEQ ID NOs: 1-476.

In certain embodiments, the rAAV genome further comprises a 5′ inverted terminal repeat (5′ ITR) nucleotide sequence 5′ of the transgene, and a 3′ inverted terminal repeat (3′ ITR) nucleotide sequence 3′ of the transgene. ITR sequences from any AAV serotype or variant thereof can be used in the rAAV genomes described herein. The 5′ and 3′ ITR can be from an AAV of the same serotype or from AAVs of different serotypes.

In certain embodiments, the rAAV genome is a single-stranded rAAV genome. In certain embodiments, the rAAV genome is a self-complementary rAAV genome.

In another aspect, the present disclosure provides an rAAV comprising a capsid comprising an AAV capsid protein, and an rAAV genome as described herein.

A capsid protein from any capsid known the art can be used in the rAAVs described herein, including, without limitation, a capsid protein from an AAV1, AAV2, AAV3, AAV4, AAV5, AAV6, AAV7, AAV8, or AAV9 serotype. The capsid protein can be from a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein, or an engineered variant thereof.

In certain embodiments, the capsid protein is from AAV8. In certain embodiments, the capsid protein is encoded by a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the nucleotide sequence set forth in SEQ ID NO: 495. In certain embodiments, the capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of amino acids 1-738 of SEQ ID NO: 496; an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of amino acids 138-738 of SEQ ID NO: 497; and/or an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of amino acids 204-738 of SEQ ID NO: 498. In certain embodiments, the capsid protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 496; an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 497; and/or an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 498.

In another aspect, the present disclosure provides compositions, e.g., pharmaceutical compositions comprising a rAAV as described herein together with a pharmaceutically acceptable excipient, adjuvant, diluent, vehicle or carrier, or a combination thereof. A “pharmaceutically acceptable carrier” includes any material which, when combined with an active ingredient of a composition, allows the ingredient to retain biological activity and without causing disruptive physiological reactions, such as an unintended immune reaction. Pharmaceutically acceptable carriers include water, phosphate buffered saline, emulsions such as oil/water emulsion, and wetting agents. Compositions comprising such carriers are formulated by well-known conventional methods such as those set forth in Remington's Pharmaceutical Sciences, current Ed., Mack Publishing Co., Easton Pa. 18042, USA; A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., 7th ed., Lippincott, Williams, & Wilkins; and Handbook of Pharmaceutical Excipients (2000) A. H. Kibbe et al., 3rd ed. Amer. Pharmaceutical Assoc.

### IV. Adeno-Associated Virus Packaging Systems

In another aspect, the instant disclosure provides packaging systems for recombinant preparation of a recombinant adeno-associated virus (rAAV) described herein. Such packaging systems generally comprise: a first nucleotide encoding one or more AAV Rep proteins; a second nucleotide encoding an AAV capsid protein as described herein; and a third nucleotide sequence comprising any of the rAAV genome sequences as described herein, wherein the packaging system is operative in a cell for enclosing the transfer genome in the capsid to form the AAV.

In certain embodiments, the packaging system comprises a first vector comprising the first nucleotide sequence encoding the one or more AAV Rep proteins and the second nucleotide sequence encoding the AAV capsid protein, and a second vector comprising the third nucleotide sequence comprising the rAAV genome. As used in the context of a packaging system as described herein, a “vector” refers to a nucleic acid molecule that is a vehicle for introducing nucleic acids into a cell (e.g., a plasmid, a virus, a cosmid, an artificial chromosome, etc.). In certain embodiments of the packaging system, the packaging system further comprises a fourth nucleotide sequence comprising one or more helper virus genes. In certain embodiments, the fourth nucleotide sequence comprises adenoviral E2, E4, and VA genes. In certain embodiments of the packaging system, the packaging system further comprises a third vector (e.g., a helper virus vector), comprising the fourth nucleotide sequence. The third vector may be an independent third vector, integral with the first vector, or integral with the second vector.

In certain embodiments, the packaging system comprises a first vector comprising the first nucleotide sequence encoding one or more AAV Rep proteins, the second nucleotide sequence encoding one or more recombinant AAV capsid protein, and the third nucleotide sequence comprising any of the rAAV genome sequences as described herein, wherein the packaging system is operative in a cell for enclosing the transfer genome in the capsid to form the AAV. In certain embodiments of the packaging system, the packaging system further comprises a fourth nucleotide sequence comprising one or more helper virus genes. In certain embodiments, the fourth nucleotide sequence comprises adenoviral E2, E4, and VA genes. In certain embodiments of the packaging system, the packaging system further comprises a second vector (e.g., a helper virus vector), comprising the fourth nucleotide sequence. The second vector may be an independent second vector, integral with the first vector.

Any AAV Rep protein can be employed in the packaging systems described herein. In certain embodiments of the packaging system, the Rep nucleotide sequence encodes an AAV2 Rep protein. Suitable AAV2 Rep proteins may include, without limitation, Rep 78/68 or Rep 68/52.

In certain embodiments of the packaging system, the helper virus is selected from the group consisting of adenovirus, herpes virus (including herpes simplex virus (HSV)), poxvirus (such as vaccinia virus), cytomegalovirus (CMV), and baculovirus. In certain embodiments of the packaging system, where the helper virus is adenovirus, the adenovirus genome comprises one or more adenovirus RNA genes selected from the group consisting of E1, E2, E4 and VA. In certain embodiments of the packaging system, where the helper virus is adenovirus, the adenovirus genome comprises one or more adenovirus RNA genes selected from the group consisting of E2, E4, and VA. In certain embodiments of the packaging system, where the helper virus is HSV, the HSV genome comprises one or more of HSV genes selected from the group consisting of UL5/8/52, ICPO, ICP4, ICP22, and UL30/UL42.

In certain embodiments of the packaging system, the vectors (e.g., first, second, and/or third vectors) are contained within one or more plasmids.

In certain embodiments of the packaging system, the first, second, and/or third vectors are contained within one or more recombinant helper viruses. In certain embodiments, the first vector and the third vector are contained within a recombinant helper virus. In certain embodiments, the second vector and the third vector are contained within a recombinant helper virus.

In a further aspect, the disclosure provides a method for recombinant preparation of an AAV as described herein, wherein the method comprises transfecting or transducing a cell with a packaging system as described herein under conditions operative for enclosing the rAAV genome in the capsid to form the rAAV as described herein. Exemplary methods for recombinant preparation of an rAAV include transient transfection (e.g., with one or more transfection plasmids containing a first, and a second, and optionally a third vector as described herein), viral infection (e.g. with one or more recombinant helper viruses, such as a adenovirus, poxvirus (such as vaccinia virus), herpes virus (including HSV, cytomegalovirus, or baculovirus, containing a first, and a second, and optionally a third vector as described herein)), and stable producer cell line transfection or infection (e.g., with a stable producer cell, such as a mammalian or insect cell, containing a Rep nucleotide sequence encoding one or more AAV Rep proteins and/or a Cap nucleotide sequence encoding one or more AAV capsid proteins, and with a rAAV genome as described herein being delivered in the form of a plasmid or a recombinant helper virus).

Accordingly, the instant disclosure provides a packaging system for preparation of an rAAV, wherein the packaging system comprises: a first nucleotide sequence encoding one or more AAV Rep proteins; a second nucleotide sequence encoding a capsid protein of any one of the AAVs described herein; a third nucleotide sequence comprising an rAAV genome sequence of any one of the AAVs described herein; and optionally a fourth nucleotide sequence comprising one or more helper virus genes (e.g., adenoviral E2, E4, and VA genes).

### V. Methods of Use

In another aspect, the present disclosure provides a method comprising introducing into a cell a polynucleotide as described herein, a vector as described herein, or a rAAV genome as described herein. In another aspect, the present disclosure provides a method comprising transducing a cell with an rAAV as described herein.

Accordingly, the present disclosure provides methods for transducing a cell. The methods generally comprise contacting the cell with an rAAV disclosed herein under conditions whereby the cell is transduced. The rAAV disclosed herein can be used to transduce cells in vitro, in vivo and ex vivo.

In another aspect, the present disclosure provides methods for delivering a transgene into a cell. The methods generally comprise comprising contacting the cell with an rAAV disclosed herein under conditions whereby the cell is transduced and the transgene is expressed.

The rAAV disclosed herein can comprise a transgene under the control of a TRE. Accordingly, in certain embodiments, the instant disclosure provides methods for expressing a transgene in a cell, the method generally comprising contacting the cell with such an rAAV under conditions whereby the cell is transduced and the transgene is expressed. The transgene can encode a polypeptide and/or an RNA molecule, as described herein. Accordingly, in certain embodiments, the instant disclosure provides methods for producing a polypeptide and/or an RNA molecule in a cell, the method generally comprising contacting the cell with such an rAAV under conditions whereby the cell is transduced and the polypeptide and/or an RNA molecule is produced.

In certain embodiments, the ligand responsive ribozyme comprised within the polynucleotide, vector, or rAAV genome is responsive to theophylline, or a derivative thereof. In the absence of theophylline, or a derivative thereof, the ligand responsive ribozyme may inhibit transgene expression by facilitating self-cleavage, leading to the cleaving of the polyadenylation sequence off of the transcript, resulting in transcript instability and degradation. In the presence of theophylline, or a derivative thereof, the ligand-bound ligand responsive ribozyme is in a conformation in which self-cleavage cannot occur, thus resulting in the transcript having an intact polyadenylation sequence, allowing for transgene expression to occur.

Accordingly, in another aspect, the present disclosure provides a method of modulating the expression of a transgene comprising: (a) introducing into a cell a polynucleotide, vector, or rAAV genome as described herein; and (b) contacting the cell of (a) with an effective amount of theophylline or derivative thereof. In another aspect, the present disclosure provides a method of modulating the expression of a transgene comprising: (a) transducing a cell with the rAAV as described herein; and (b) contacting the cell of (a) with an effective amount of theophylline or derivative thereof. In certain embodiments, contacting the cell of (a) with an effective amount of theophylline or derivative thereof increases expression of the transgene.

In another aspect, the present disclosure provides a method of modulating the expression of a transgene in a subject, comprising: (a) introducing into an isolated cell a polynucleotide as described herein, thereby generating a modified cell; (b) administering the modified cell to a subject; and (c) administering theophylline or derivative thereof to the subject at an amount effective to induce expression of the transgene. In certain embodiments, the isolated cell is an immune cell. In certain embodiments, the isolated cell is an immune cell autologous to the subject. In certain embodiments, the isolated cell is a T cell, NK cell, NKT cell, or precursor thereof.

In another aspect, the present disclosure provides a method of modulating the expression of a transgene in a subject, comprising administering theophylline or a derivative thereof to the subject at an amount effect to induce expression of the transgene, wherein the transgene is comprised within a polynucleotide, a vector, or a rAAV genome as described herein.

In another aspect, the present disclosure provides a method of modulating the expression of a transgene in a subject, comprising administering theophylline or a derivative thereof to the subject at an amount effect to induce expression of the transgene, wherein the transgene is comprised within a recombinant cell as described herein, and the recombinant cell has been administered to the subject.

In certain embodiments, the transgene comprises a suicide gene. In certain embodiments where the transgene comprises a suicide gene, the present disclosure provides methods of selectively ablating a cell, comprising (a) introducing into a cell a polynucleotide comprising a suicide gene as described herein; (b) contacting the cell of (a) with an effective amount of theophylline or derivative thereof, thereby increasing expression of the suicide gene; and (c) contacting the cell with an agent at an amount effective to ablate the cell.

The suicide gene can be any suicide gene known in the art that when expressed by a cell, confers sensitivity to an agent, e.g., a drug, such that when the cell expressing the suicide gene is contacted with or exposed to the agent, it causes the cell to die. Such suicide genes include, without limitation, HSV-TK, iCasp9, a sequence comprising a minimal epitope of CD20, and truncated EGFR.

Various theophylline derivatives are known in the art. In certain embodiments, the theophylline derivative is aminophylline or dyphylline.

In certain embodiments, the cell is in a subject and the polynucleotide, vector, or rAAV is administered to the subject. The polynucleotide, vector, or rAAV disclosed herein can be administered to a subject by all suitable routes, including, without limitation, intravenously, intraperitoneally, subcutaneously, intramuscularly, intrathecally, or intradermally.

In certain embodiments, the subject is a member of any mammalian or non-mammalian species. Suitable subjects include, without limitation, humans, non-human primates, canines, felines, ungulates (e.g., equine, bovine, swine (e.g., pig)), avians, rodents (e.g., rats, mice), and other subjects. In certain embodiments, the subject is human. In certain embodiments, the subject is canine. In certain embodiments, the subject is feline. In certain embodiments, the subject is equine.

In another aspect, the present disclosure provides the polynucleotide, vector, rAAV genome, or rAAV as described herein for use in medicine. In another aspect, the present disclosure provides the polynucleotide, vector, rAAV genome, or rAAV as described herein for use as therapy. In another aspect, the present disclosure provides the polynucleotide, vector, rAAV genome, or rAAV as described herein for use as a medicament.

### VI. EXAMPLES

The following examples are offered by way of illustration, and not by way of limitation.

### Example 1: Identification of Lead Ribozymes for Aptazyme Variant Library

To develop novel synthetic aptazyme switches that possess tight control on gene expression and improved dynamic ranges, different types of ribozymes were tested, with or without an aptamer attached thereto. Aptazymes were generated by attaching a theophylline responsive aptamer to the ribozymes, N107-v1 (Yen L, et al., Nature, 2004, 431 (7007): 471-476), STRSV (Prody G A, et al., Science, 1986, 231 (4745): 1577-1580), sTRSVH2 (Zhong G, et al., Nat Biotechnol, 2020, 38 (2): 169-175), T3H38 (Zhong G, et al., Nat Biotechnol, 2020, 38 (2): 169-175), and T3H48 (Zhong G, et al., Nat Biotechnol, 2020, 38 (2): 169-175). Theo-CAUAA (Xiang J S, et al., Nat Commun, 2019, 10 (1): 4327) as a positive control. The theophylline responsive aptamer was attached to one of the loops of the above ribozymes to create the aptazyme version (designated as “ribozyme name-A”) to test alongside the ribozyme only version (designated as “ribozyme name-R”). Table 1 provides a brief description of the various ribozymes with or without aptamers, and their corresponding sequences.

TABLE 1
Ribozyme and aptazyme sequences and descriptions.

| | | SEQ | |
|---|---|---|---|
| Name | Sequence* | ID NO | Description |
| STRSV-R-m | GCTGTCACCGGATGTGCTTTC | 477 | Negative control; non- |
| | CGGTACGTGAGGTCCGTGAGG | | cleaving variant of |
| | ACGAAACAGC | | STRSV ribozyme |
| Theo-CAUAA-S | GCTGTCACCggaataccagca | 478 | Positive control; |
| | tcgtcttgatgcccttggaag | | theophylline-sensitive |
| | tccGGTCTGATGAGTCCCATA | | aptazyme |
| | AGGACGAAACAGC | | |
| N107-v1-R | CTGAGGTGCAGGTACATCCAG | 479 | Type-I hammerhead |
| | CTGACGAGTCCCAAATAGGAC | | ribozyme |
| | GAAACGCGCTTCGGTGCGTCC | | |
| | TGGATTCCACTGCTATCCAC | | |
| N107-v1-R-A | CTGAGGTGCAGGTACATCCAG | 480 | N107-v1-R with a |
| | CTGACGAGTCCCAAATAGGAC | | theophylline aptamer |
| | GAAACGCggaataccagcatc | | |
| | gtcttgatgcccttggaagtc | | |
| | CGCGTCCTGGATTCCACTGCT | | |
| | ATCCAC | | |
| STRSV-H1-R | GGGAGCCCTGTCACCGGATGT | 481 | Type-III hammerhead |
| | GCTTTCCGGTCTGATGAGTCC | | ribozyme with longer |
| | GTGAGGACGAAACAGGGCTCC | | arm |
| | C | | |
| STRSV-H2-R | GCGCGTCACCGGATGTGCTTT | 482 | Type-III hammerhead |
| | CCGGTCTGATGAGTCCGTGAG | | ribozyme with arm |
| | GACGAAACGCGC | | from T3H48 ribozyme |
| STRSV-H2-R-A | GCGCGTCACCGgaataccagc | 483 | STRSV-H2-R with a |
| | atcgtcttgatgcccttggaa | | theophylline aptamer |
| | gTCCGGTCTGATGAGTCCGTG | | |
| | AGGACGAAACGCGC | | |
| T3H48-R | GCGCGTCCTGGATTCGCGGAA | 484 | Type-III hammerhead |
| | ACGCGTACATCCAGCTGACGA | | ribozyme |
| | GTCCCAAATAGGACGAAACGC | | |
| | GC | | |
| T3H48-A-S | GCGCGTCCTGGATTCGCGgga | 485 | Type-III hammerhead |
| | ataccagcatcgtcttgatgc | | ribozyme with a |
| | ccttggaagtccCGCGTACAT | | theophylline aptamer |
| | CCAGCTGACGAGTCCCAAATA | | |
| | GGACGAAACGCGC | | |
| T3H38-A | GCGCGTCCTGGATTCGagtga | 486 | Type-III hammerhead |
| | taccagcatcgtcttgatgcc | | ribozyme with a |
| | cttggcagcactCGTACATCC | | theophylline aptamer |
| | AGCTGACGAGTCCCAAATAGG | | |
| | ACGAAACGCGC | | |
| T3H48-STRSV-R | GCGCGTCCTGGATTCGCGGAA | 487 | Two type-III ribozymes |
| | ACGCGTACATCCAGCTGACGA | | fused with an 18 bp gap |
| | GTCCCAAATAGGACGAAACGC | | sequence |
| | GCtagtactgttctgtacgtG | | |
| | CGCGTCACCGGATGTGCTTTC | | |
| | CGGTCTGATGAGTCCGTGAGG | | |
| | ACGAAACGCGC | | |

*Upper-case letters indicate the ribozyme sequence while lower-case letters indicate the aptamer sequence. Underlined lower-case letters indicate a gap sequence between ribozymes.

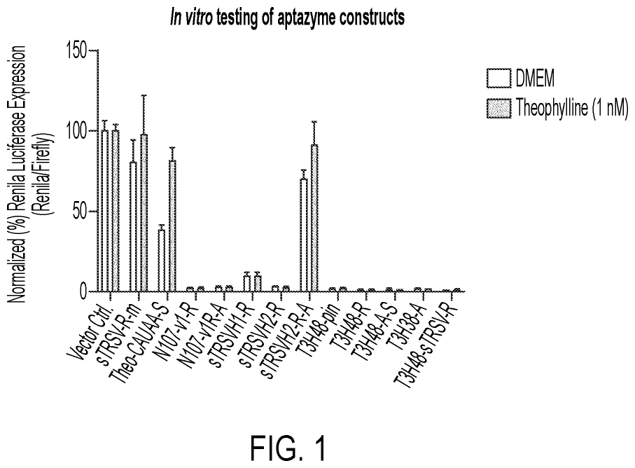

After synthesizing the sequences as gBlocks, the sequences were cloned into a psiCHECK™-2 dual luciferase (Firefly and Renilla luciferase) mammalian expression plasmid in the 3′ UTR of the Renilla luciferase gene. The cloned and sequence-verified plasmids were then transfected into HEK293-T cells in a 96-well plate using PEI reagents. After 12 hours post-transfection, DMEM media with or without 1 mM theophylline was added. 24 hours post-transfection, a Dual-Glo Luciferase Assay (Promega) was performed for quantitation of luminescent signal due to luciferase. Luciferase expression was measured as a function of the activity of Renilla luciferase normalized to the activity of Firefly luciferase (FIG. 1).

As shown in FIG. 1, expression of luciferase from the plasmid containing the negative control non-cleaving sTRSV variant (sTRSV-R-m) was high, both in the presence and absence of theophylline. By contrast, expression of luciferase from the plasmid containing the positive control Theo-CAUAA-S was lower in the absence of theophylline, but increased in the presence of theophylline. While this indicates that the aptamer is ligand-responsive, as expected, the relatively leaky expression (i.e., the relatively high expression of luciferase in the absence theophylline) indicates inefficient self-cleavage via the ribozyme component of the aptamer in the ligand-free state. This inefficient self-cleavage limits the dynamic range of expression in the ligand-bound state.

FIG. 1 also demonstrates that plasmid containing the T3H48 ribozyme only (T3H48-R) had the tightest control of luciferase expression. However, plasmid containing the aptazyme version of T3H48 (T3H48-A-S) did not result in increased luciferase expression in the presence of theophylline. Due to this, the T3H48 ribozyme was selected as a candidate for a systematic screen of aptamer sequences inserted into positions within the ribozyme to identify resulting aptazymes that result in tight control of expression in the absence of theophylline, but a wide dynamic range of expression in the presence of theophylline.

### Example 2: Generation and In Vitro Screening of Aptazyme Variant Library

Based on the findings from Example 1, an aptazyme variant library was generated from the T3H48 ribozyme with one of seven variant theophylline aptamers inserted sequentially after each nucleotide of the DNA sequence encoding the T3H48 ribozyme sequence. This resulted in a library of 463 unique aptazymes. The seven variant theophylline aptamers vary in their communication channels, which are the ribonucleotides of the aptamer that attach to the ribozyme sequence (see, Table 2).

TABLE 2
Aptamer sequences.

| | | SEQ ID |
|---|---|---|
| Name | Sequence | NO |
| Aptamer 1 | GGAATACCAGCATCGTCTTGATGCCCTTGGAAGTCC | 488 |
| Aptamer 2 | CGGAATACCAGCATCGTCTTGATGCCCTTGGAAGTCCG | 489 |
| Aptamer 3 | CCGGAATACCAGCATCGTCTTGATGCCCTTGGAAGTCCGG | 490 |
| Aptamer 4 | AGAATACCAGCATCGTCTTGATGCCCTTGGAAGTCT | 491 |
| Aptamer 5 | GAGAATACCAGCATCGTCTTGATGCCCTTGGAAGTCTC | 492 |
| Aptamer 6 | CGAGAATACCAGCATCGTCTTGATGCCCTTGGAAGTCTCG | 493 |
| Aptamer 7 | AGTGATACCAGCATCGTCTTGATGCCCTTGGCAGCACT | 494 |

Additionally, 14 control riboswitches were included for comparison (five positive control aptazymes derived from Xiang J S, et al., Nat Commun, 2019, 10 (1): 4327; five aptazymes with mutations in the ribozyme core to deactivate the self-cleavage activity; two inactivated ribozymes without any aptamer attached; and two T3H48 ribozymes without any aptamer attached), bringing the total library size to 477. The library was barcoded with unique sequences of nine nucleotides flanked by forward and reverse primer amplification sequences.

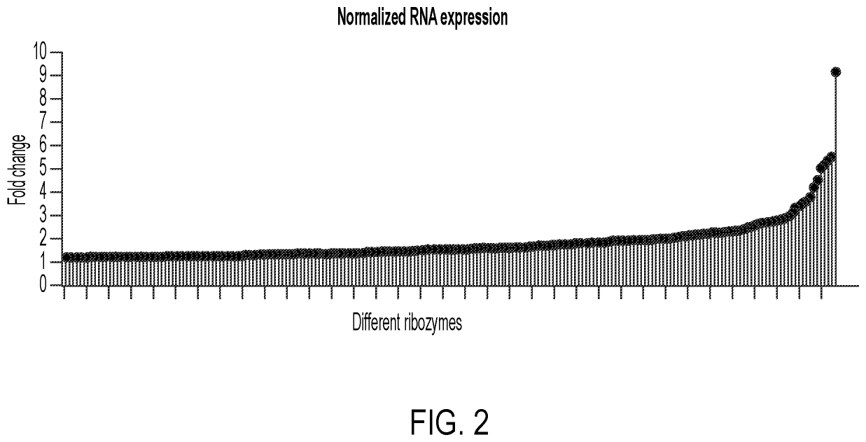

The aptazyme library was then PCR amplified and cloned into the psiCHECK™-2 dual luciferase vector in the 3′ UTR of the Renilla luciferase gene. The cloned and sequence verified plasmids were then transfected into HEK293-T cells in a 6-well plate format using PEI transfection reagents. Twenty-four hours post-transfection, DMEM media with or without 1 mM theophylline was added. The next day, the cells were isolated followed by DNA and RNA extraction using standard methods. The purified RNA was then converted to cDNA and the barcoded portion of the aptazyme library was PCR amplified. Similarly, the DNA was PCR amplified and all the samples were sequenced. All experimental conditions (with theophylline and without theophylline) were performed in triplicate. The read count for each barcode was determined with a custom build code and the data was normalized to identify the un-cleaved fractions of the Renilla luciferase RNA from cells that were cultured in the presence or absence of theophylline for each aptazyme, which was used to obtain the fold-change between ON- and OFF-states (FIG. 2).

As demonstrated by FIG. 2, the fold-change in Renilla luciferase RNA expression in cells cultured in the presence of theophylline (“ON state”) over luciferase RNA expression in cells cultured in the absence of theophylline (“OFF state”) ranged from slightly over 1 to over 5. Fifty of the aptazymes tested had at least a 2-fold change in RNA expression between the ON state and the OFF state. These fifty aptazyme sequences are provided in Table 3 below.

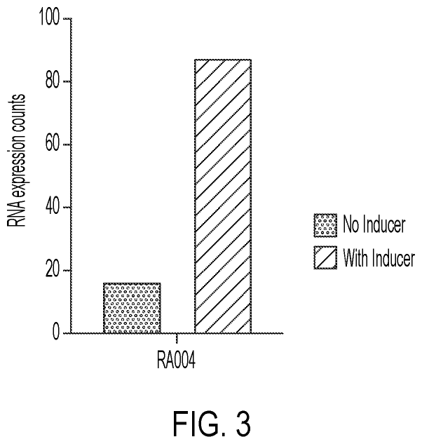

FIG. 3 demonstrates the difference in RNA expression counts for cells comprising the exemplary aptazyme designated RA004 cultured in the absence or presence of theophylline as inducer. These results suggest that the aptazymes described herein tightly control RNA expression in the OFF state but lead to robust RNA expression in the ON state, which has broad applications for the temporal and spatial regulation of expression constructs, including those used in vivo for gene therapies.

TABLE 3
Aptazyme sequences.

| SEQ | | | Fold |
|---|---|---|---|
| ID NO | ID | DNA Sequence^{α} | change^{β} |
| 1 | RA001 | GCGCGTCCTGGATTCGCGGAAACGCGTACATCCAGCTGAC | >6 |
| | | GAGTCCCAAATAGGACGAAACGCGCagtgataccagcatc | |
| | | gtcttgatgcccttggcagcact | |
| 2 | RA002 | GCGCGTCCTGGATTCGCGGAAACGCGTACATCCAGCTGAC | >6 |
| | | GAGTCCCAAATAGGACGAAACGAgaataccagcatcgtct | |
| | | tgatgcccttggaagtcTCGCGCGC | |
| 3 | RA003 | GCGCGTCCTGCggaataccagcatcgtcttgatgcccttg | 5.5 |
| | | gaagtccGGATTCGCGGAAACGCGTACATCCAGCTGACGA | |
| | | GTCCCAAATAGGACGAAACGCGC | |
| 4 | RA004 | GCGCGTCCTGGATTCGCGGAAACGCGTACATCCAGCTGAC | 5.3 |
| | | GAGTCCCAAATAGGCCggaataccagcatcgtcttgatgc | |
| | | ccttggaagtccGGACGAAACGCGC | |
| 5 | RA005 | GCGCGTCCTGGATTCGCGGAAACCggaataccagcatcgt | 5.2 |
| | | cttgatgcccttggaagtccGGCGCGTACATCCAGCTGAC | |
| | | GAGTCCCAAATAGGACGAAACGCGC | |
| 6 | RA006 | GCGCGTCCTGGATTCGCGGAAACGCGTACATCCAGCTGAC | 5.0 |
| | | GAGTCCCAAATAGGACGAAACGagtgataccagcatcgtc | |
| | | ttgatgcccttggcagcactCGC | |
| 7 | RA007 | GCGCGTCCTGGATTCGCGGAAACGCGTACATCCAGCTGAC | 4.5 |
| | | GAGTCCCAAATAGGACGAAACGCGAgaataccagcatcgt | |
| | | cttgatgcccttggaagtcTCGCGC | |
| 8 | RA008 | GCGCGTCCTGGATTCGCGGAAACggaataccagcatcgtc | 4.2 |
| | | ttgatgcccttggaagtccGCGCGTACATCCAGCTGACGA | |
| | | GTCCCAAATAGGACGAAACGCGC | |
| 9 | RA009 | GCGCGTCCTGGATTCGCGGAAACGCGTACATCCAGCTGAC | 3.8 |
| | | GAGTCCCAAATAGGACGACCggaataccagcatcgtcttg | |
| | | atgcccttggaagtccGGAACGCGC | |
| 10 | RA010 | GCGCGTCCTGGACGAgaataccagcatcgtcttgatgccc | 3.6 |
| | | ttggaagtcTCGTTCGCGGAAACGCGTACATCCAGCTGAC | |
| | | GAGTCCCAAATAGGACGAAACGCGC | |
| 11 | RA011 | GCGCGTCCTGGATTCGCGGAAACGCGTACATCCAGCTGAC | 3.6 |
| | | GAGTCCCggaataccagcatcgtcttgatgcccttggaag | |
| | | tccGCAAATAGGACGAAACGCGC | |
| 12 | RA012 | GCGCGTCCTGGATTCGCGGAAACAgaataccagcatcgtc | 3.4 |
| | | ttgatgcccttggaagtcTGCGTACATCCAGCTGACGAGT | |
| | | CCCAAATAGGACGAAACGCGC | |
| 13 | RA013 | GCGCGTCCTGGATTCGCGGAAACGCGTACATCCAGCTGAC | 3.3 |
| | | GAGTCCCAAATAGGACGAAACGAgaataccagcatcgtct | |
| | | tgatgcccttggaagtcTCGC | |
| 14 | RA014 | GCGCGTCCTGGATTCGCGGAAACGCGTACATCCAGCTGAC | 3.1 |
| | | GAGTCCCAAATAGGACGAAACggaataccagcatcgtctt | |
| | | gatgcccttggaagtccGCGC | |
| 15 | RA015 | GCGCGTCCTGGATTCGCGGCggaataccagcatcgtcttg | 3.0 |
| | | atgcccttggaagtccGAAACGCGTACATCCAGCTGACGA | |
| | | GTCCCAAATAGGACGAAACGCGC | |
| 16 | RA016 | GCGCGTCCTGGATGAgaataccagcatcgtcttgatgccc | 2.8 |
| | | ttggaagtcTCTCGCGGAAACGCGTACATCCAGCTGACGA | |
| | | GTCCCAAATAGGACGAAACGCGC | |
| 17 | RA017 | GCGCGTCCTGGCggaataccagcatcgtcttgatgccctt | 2.8 |
| | | ggaagtccGATTCGCGGAAACGCGTACATCCAGCTGACGA | |
| | | GTCCCAAATAGGACGAAACGCGC | |
| 18 | RA018 | GCGCGTCCTGGATTCGCGGAAACGCGTACATCCAGCTGAC | 2.8 |
| | | GAGTCCCAAagtgataccagcatcgtcttgatgcccttgg | |
| | | cagcactATAGGACGAAACGCGC | |
| 19 | RA019 | GCGCGTCCTGGATTCGCGGAAACGCGTACATCCAGCTGAC | 2.7 |
| | | GggaataccagcatcgtcttgatgcccttggaagtccAGT | |
| | | CCCAAATAGGACaAAACGCGC | |
| 20 | RA020 | GCGCGTCCTGGATTCGCGGAAACGCGTACATCggaatacc | 2.7 |
| | | agcatcgtcttgatgcccttggaagtccGCCAGCTGACGA | |
| | | GTCCCAAATAGGACGAAACGCGC | |
| 21 | RA021 | GCGCGTCCTGGATTCGCGGAAACGCGTACATCCAGCTGAC | 2.7 |
| | | GAGTCCCAAATAGagtgataccagcatcgtcttgatgccc | |
| | | ttggcagcactGACGAAACGCGC | |
| 22 | RA022 | GCGCGTCCTGGATTCGCGGAAACGCGTACAagtgatacca | 2.6 |
| | | gcatcgtcttgatgcccttggcagcactTCCAGCTGACGA | |
| | | GTCCCAAATAGGACGAAACGCGC | |
| 23 | RA023 | GCGCGTCCTGGATTCGCGGAAACGCGTGAgaataccagca | 2.6 |
| | | tcgtcttgatgcccttggaagtcTCACATCCAGCTGACGA | |
| | | GTCCCAAATAGGACGAAACGCGC | |
| 24 | RA024 | GCGCGTCCTGGATTCGCGGAAACGCGTACATCCAGCTGAC | 2.6 |
| | | GAGTCCCAAATAGGACagtgataccagcatcgtcttgatg | |
| | | cccttggcagcactGAAACGCGC | |
| 25 | RA025 | GCGCGTCCCGAgaataccagcatcgtcttgatgcccttgg | 2.5 |
| | | aagtcTCGTGGATTCGCGGAAACGCGTACATCCAGCTGAC | |
| | | GAGTCCCAAATAGGACGAAACGCGC | |
| 26 | RA026 | GCGCGTCCTGGATTCGCGGAAACGCGTACATCCAGCTGAC | 2.4 |
| | | GAGTCCGAgaataccagcatcgtcttgatgcccttggaag | |
| | | tcTCGCCAAATAGGACGAAACGCGC | |
| 27 | RA027 | GCGCGTCCTGGATTCGCGGAAACGCGTACagtgataccag | 2.4 |
| | | catcgtcttgatgcccttggcagcactATCCAGCTGACGA | |
| | | GTCCCAAATAGGACGAAACGCGC | |
| 28 | RA028 | GCGCGTCCTGGATTCGCGGAAACGCGTACATCCAGCTGAC | 2.3 |
| | | GACggaataccagcatcgtcttgatgcccttggaagtccG | |
| | | GTCCCAAATAGGACGAAACGCGC | |
| 29 | RA029 | GCGCGTCCTGGATTCGCGGAAACGggaataccagcatcgt | 2.3 |
| | | cttgatgcccttggaagtccCGTACATCCAGCTGACGAGT | |
| | | CCCAAATAGGACGAAACGCGC | |
| 30 | RA030 | GCGCGTCCTGGATTCGCGGAAACGCGTACATCCAGCTGAC | 2.3 |
| | | GAGTCCCAAATAGGACGAAACGAgaataccagcatcgtct | |
| | | tgatgcccttggaagtcTCGCGC | |
| 31 | RA031 | GCGCGTCCGAgaataccagcatcgtcttgatgcccttgga | 2.3 |
| | | agtcTCTGGATTCGCGGAAACGCGTACATCCAGCTGACGA | |
| | | GTCCCAAATAGGACGAAACGCGC | |
| 32 | RA032 | GCGCGTCCTGGATTCGCGGAAAagtgataccagcatcgtc | 2.3 |
| | | ttgatgcccttggcagcactCGCGTACATCCAGCTGACGA | |
| | | GTCCCAAATAGGACGAAACGCGC | |
| 33 | RA033 | GCGCGTCCTGGATTCGCGGAAACGCGTACATCCAGCTGAC | 2.3 |
| | | GAGTCCCAAATCCggaataccagcatcgtcttgatgccct | |
| | | tggaagtccGGAGGACGAAACGCGC | |
| 34 | RA034 | GCGCGTCCTGGATTCGCGGAAACGCGTACATCCAGCTGAC | 2.3 |
| | | GAGAgaataccagcatcgtcttgatgcccttggaagtcTC | |
| | | GTCCCAAATAGGACGAAACGCGC | |
| 35 | RA035 | GCGCGTCCTGGATTCGCGGAAACGCGTACATCCAGCTGAC | 2.2 |
| | | GAGTCCCAAATAGGACGAAACggaataccagcatcgtctt | |
| | | gatgcccttggaagtccGCGCGC | |
| 36 | RA036 | GCGCGTCCTGGATTCGCGGAAACGCGTACATCCAGCTGAC | 2.2 |
| | | GAGTCCCAAATAGGCGAgaataccagcatcgtcttgatgc | |
| | | ccttggaagtcTCGACGAAACGCGC | |
| 37 | RA037 | GCGCGTCCTGGATTCGCCggaataccagcatcgtcttgat | 2.2 |
| | | gcccttggaagtccGGGAAACGCGTACATCCAGCTGACGA | |
| | | GTCCCAAATAGGACGAAACGCGC | |
| 38 | RA038 | GCGCGTCCTGGATTCGCGGAAACGCGTACATCAgaatacc | 2.2 |
| | | agcatcgtcttgatgcccttggaagtcTCAGCTGACGAGT | |
| | | CCCAAATAGGACGAAACGCGC | |
| 39 | RA039 | GCGCGTCCTGGATTCGCGGAAACGCGTACATCggaatacc | 2.2 |
| | | agcatcgtcttgatgcccttggaagtccCAGCTGACGAGT | |
| | | CCCAAATAGGACGAAACGCGC | |
| 40 | RA040 | GCGCGTCCTGGATTCGCGAgaataccagcatcgtcttgat | 2.1 |
| | | gcccttggaagtcTGAAACGCGTACATCCAGCTGACGAGT | |
| | | CCCAAATAGGACGAAACGCGC | |
| 41 | RA041 | GCGCGTCCTGGATTCGCGGAAACGCGagtgataccagcat | 2.1 |
| | | cgtcttgatgcccttggcagcactTACATCCAGCTGACGA | |
| | | GTCCCAAATAGGACGAAACGCGC | |
| 42 | RA042 | GCGCGTCCTGGATTCGCGGAAACGCGTACATCCAAgaata | 2.1 |
| | | ccagcatcgtcttgatgcccttggaagtcTGCTGACGAGT | |
| | | CCCAAATAGGACGAAACGCGC | |
| 43 | RA043 | GCGCGTCCTCGAgaataccagcatcgtcttgatgcccttg | 2.1 |
| | | gaagtcTCGGGATTCGCGGAAACGCGTACATCCAGCTGAC | |
| | | GAGTCCCAAATAGGACGAAACGCGC | |
| 44 | RA044 | GCGGAgaataccagcatcgtcttgatgcccttggaagtcT | 2.1 |
| | | CCGTCCTGGATTCGCGGAAACGCGTACATCCAGCTGACGA | |
| | | GTCCCAAATAGGACGAAACGCGC | |
| 45 | RA045 | GAgaataccagcatcgtcttgatgcccttggaagtcTCGC | 2.0 |
| | | GCGTCCTGGATTCGCGGAAACGCGTACATCCAGCTGACGA | |
| | | GTCCCAAATAGGACGAAACGCGC | |
| 46 | RA046 | GCGCGTCCTGGATTCGCGGAAACGCGTACATCCAGCTGAC | 2.0 |
| | | GAGTCCggaataccagcatcgtcttgatgcccttggaagt | |
| | | CCGCCAAATAGGACGAAACGCGC | |
| 47 | RA047 | GCGCGTCCTGGATTCGCGGAAACGCGTACATCCAGCTGAC | 2.0 |
| | | GAGTCCCAggaataccagcatcgtcttgatgcccttggaa | |
| | | gtccAATAGGACGAAACGCGC | |
| 48 | RA048 | GCGCGTCCTGGATTCGCGGAAACGCGTACATCCAGCTGAC | 2.0 |
| | | GCGAgaataccagcatcgtcttgatgcccttggaagtcTC | |
| | | GAGTCCCAAATAGGACGAAACGCGC | |
| 49 | RA049 | GCGCGTCCTGGATTCGCGGAAACGCGTACATAgaatacca | 2.0 |
| | | gcatcgtcttgatgcccttggaagtcTCCAGCTGACGAGT | |
| | | CCCAAATAGGACGAAACGCGC | |
| 50 | RA050 | GCGCGTCCTGGATTCGCGGAAACGCGTACATCCAGCTGAC | 2.0 |
| | | GAgaataccagcatcgtcttgatgcccttggaagtcTCGC | |
| | | GAGTCCCAAATAGGACGAAACGCGC | |
| 51 | RA051 | GCGCGTCCTGGATTCGCGGAAACGCGTACATCCAGCTGAC | 2.0 |
| | | GAGTCCCAAATAGGACGAAACCGAgaataccagcatcgtc | |
| | | ttgatgcccttggaagtcTCGGCGC | |
| 52 | RA052 | GCGCGTCCTGGATTCGCGGAAACGCGTACATCCAGCTGAC | 2.0 |
| | | GAGagtgataccagcatcgtcttgatgcccttggcagcac | |
| | | tTCCCAAATAGGACGAAACGCGC | |
| 53 | RA053 | GCGCGTCCTGGATTCGCGGAAACGCGTACATCCAGagtga | 2.0 |
| | | taccagcatcgtcttgatgcccttggcagcactCTGACGA | |
| | | GTCCCAAATAGGACGAAACGCGC | |
| 54 | RA054 | GagtgataccagcatcgtcttgatgcccttggcagcactC | 2.0 |
| | | GCGTCCTGGATTCGCGGAAACGCGTACATCCAGCTGACGA | |
| | | GTCCCAAATAGGACGAAACGCGC | |
| 55 | RA055 | GCGCGTCCTGGATTCGCGGCGAgaataccagcatcgtctt | 2.0 |
| | | gatgcccttggaagtcTCGAAACGCGTACATCCAGCTGAC | |
| | | GAGTCCCAAATAGGACGAAACGCGC | |
| 56 | RA056 | GCGCGTCCTGGATTCGCGGAAACGCGTACATCCAGCTGAC | 2.0 |
| | | GAGTCCCGAgaataccagcatcgtcttgatgcccttggaa | |
| | | gtcTCAAATAGGACGAAACGCGC | |
| 57 | RA057 | GCGCGTCCTGGATTCGCGGAAACGCGTACATCCAGCTGAC | 2.0 |
| | | GAGTCCCAAATAGGACAgaataccagcatcgtcttgatgc | |
| | | ccttggaagtcTGAAACGCGC | |
| 58 | RA058 | GCGCGTCCTGGATTCGCGGAAACGCGTACATCCAGAgaat | 2.0 |
| | | accagcatcgtcttgatgcccttggaagtcTCGCTGACGA | |
| | | GTCCCAAATAGGACGAAACGCGC | |
| 59 | RA059 | GCGCGTCCTGGATTCCCggaataccagcatcgtcttgatg | 1.9 |
| | | cccttggaagtccGGGCGGAAACGCGTACATCCAGCTGAC | |
| | | GAGTCCCAAATAGGACGAAACGCGC | |
| 60 | RA060 | GCGCGTCCTGGATTCGCGGAAACGCGTACATCCAGCTGAC | 1.9 |
| | | GAGTCCCAAATAGGACGAAACGCGggaataccagcatcgt | |
| | | cttgatgcccttggaagtccC | |
| 61 | RA061 | GCGCGTCCTGGATTCGCGGAAACGCGTACATCCAGCTGAC | 1.9 |
| | | GAGTCCCAAATAGGACGAAACGCGCggaataccagcatcg | |
| | | tcttgatgcccttggaagtcc | |
| 62 | RA062 | GCGCGTCCTGGATTCGCGGAAACGCCCggaataccagcat | 1.9 |
| | | cgtcttgatgcccttggaagtccGGGTACATCCAGCTGAC | |
| | | GAGTCCCAAATAGGACGAAACGCGC | |
| 63 | RA063 | GCGCGTCCTGGATTCagtgataccagcatcgtcttgatgc | 1.9 |
| | | ccttggcagcactGCGGAAACGCGTACATCCAGCTGACGA | |
| | | GTCCCAAATAGGACGAAACGCGC | |
| 64 | RA064 | GCGCGTCCTGGATTCGCGGAAACGCGTACAgaataccagc | 1.9 |
| | | atcgtcttgatgcccttggaagtcTATCCAGCTGACGAGT | |
| | | CCCAAATAGGACGAAACGCGC | |
| 65 | RA065 | GCTGTCACCGGAATACCAGCATCGTCTTGATGCCCTTGGA | 1.9 |
| | | AGTCCGGTCTGATGAGTCCCAGAAGGACGAAACAGC | |
| 66 | RA066 | GCGCGTCCTGGATTCGCGGAAAgaataccagcatcgtctt | 1.9 |
| | | gatgcccttggaagtcTACGCGTACATCCAGCTGACGAGT | |
| | | CCCAAATAGGACGAAACGCGC | |
| 67 | RA067 | GCGCGTCCTGGATTCGCGGAAACGCGTACATCCagtgata | 1.8 |
| | | ccagcatcgtcttgatgcccttggcagcactAGCTGACGA | |
| | | GTCCCAAATAGGACGAAACGCGC | |
| 68 | RA068 | GCGCGTCCTGGATTCGCCCggaataccagcatcgtcttga | 1.8 |
| | | tgcccttggaagtccGGGGAAACGCGTACATCCAGCTGAC | |
| | | GAGTCCCAAATAGGACGAAACGCGC | |
| 69 | RA069 | GCGCGTCCTGGATTCGCGGAAACGCGTACATCCAGCTGAC | 1.8 |
| | | GAGTCCCggaataccagcatcgtcttgatgcccttggaag | |
| | | tccAAATAGGACGAAACGCGC | |
| 70 | RA070 | GCGCGTCCTGGATTCGCGGAAACGCGTACATCCAGCTGag | 1.8 |
| | | tgataccagcatcgtcttgatgcccttggcagcactACGA | |
| | | GTCCCAAATAGGACGAAACGCGC | |
| 71 | RA071 | GCGCGTCCTGGATTCGCGGAAACGCGTACATCCAGCTGAC | 1.8 |
| | | GAGTCCCAAATAGGACGAAACGCggaataccagcatcgtc | |
| | | ttgatgcccttggaagtccGCGC | |
| 72 | RA072 | GCGCGTCCTGGATTCGCGGAAACGCGTACCGAgaatacca | 1.8 |
| | | gcatcgtcttgatgcccttggaagtcTCGATCCAGCTGAC | |
| | | GAGTCCCAAATAGGACGAAACGCGC | |
| 73 | RA073 | GCGCGTCCTGGATTCGCGGAAACGCGTACACggaatacca | 1.8 |
| | | gcatcgtcttgatgcccttggaagtccGTCCAGCTGACGA | |
| | | GTCCCAAATAGGACGAAACGCGC | |
| 74 | RA074 | GCGCGTCCTGGATTCGCGGAAACGCGTACATCCAGCTGAC | 1.8 |
| | | GAGTCAgaataccagcatcgtcttgatgcccttggaagtc | |
| | | TCCAAATAGGACGAAACGCGC | |
| 75 | RA075 | GCGCGTCCTGGATTCGCGGAAACGCGTACATCCAGCTGAC | 1.8 |
| | | GAGTCCCAAATAGGACGAAagtgataccagcatcgtcttg | |
| | | atgcccttggcagcactACGCGC | |
| 76 | RA076 | GCGCGTCCTGGATTCGCGGGAgaataccagcatcgtcttg | 1.8 |
| | | atgcccttggaagtcTCAAACGCGTACATCCAGCTGACGA | |
| | | GTCCCAAATAGGACGAAACGCGC | |
| 77 | RA077 | GCGCGTCCagtgataccagcatcgtcttgatgcccttggc | 1.8 |
| | | agcactTGGATTCGCGGAAACGCGTACATCCAGCTGACGA | |
| | | GTCCCAAATAGGACGAAACGCGC | |
| 78 | RA078 | GCGCGTCCTGGATTCGCGGAAACGCGTACATCCAGCTGAC | 1.8 |
| | | GAGTCCCAAATAGGACGAAACGCCGAgaataccagcatcg | |
| | | tcttgatgcccttggaagtcTCGGC | |
| 79 | RA079 | GCGCGTCCTGGATTCGCGGAAACGCGTACATCCAGCGAga | 1.8 |
| | | ataccagcatcgtcttgatgcccttggaagtcTCGCTGAC | |
| | | GAGTCCCAAATAGGACGAAACGCGC | |
| 80 | RA080 | GCGCGTCCTGGATTCGGAgaataccagcatcgtcttgatg | 1.7 |
| | | cccttggaagtcTCCGGAAACGCGTACATCCAGCTGACGA | |
| | | GTCCCAAATAGGACGAAACGCGC | |
| 81 | RA081 | GCGCGTCCTGGATTCGCGGAAACGCGTACATCCAGCTGAC | 1.7 |
| | | GAGTCCCAAggaataccagcatcgtcttgatgcccttgga | |
| | | agtccATAGGACGAAACGCGC | |
| 82 | RA082 | GCGCGTCCTGGATTCGAgaataccagcatcgtcttgatgc | 1.7 |
| | | ccttggaagtcTCGGAAACGCGTACATCCAGCTGACGAGT | |
| | | CCCAAATAGGACGAAACGCGC | |
| 83 | RA083 | GCGCGTCCggaataccagcatcgtcttgatgcccttggaa | 1.7 |
| | | gtccTGGATTCGCGGAAACGCGTACATCCAGCTGACGAGT | |
| | | CCCAAATAGGACGAAACGCGC | |
| 84 | RA084 | GCGCGTCCTGGAGAgaataccagcatcgtcttgatgccct | 1.7 |
| | | tggaagtcTCTTCGCGGAAACGCGTACATCCAGCTGACGA | |
| | | GTCCCAAATAGGACGAAACGCGC | |
| 85 | RA085 | GCGCGTCCTGGATTCGCGGAAACGCGTACATCCAgaatac | 1.7 |
| | | cagcatcgtcttgatgcccttggaagtcTAGCTGACGAGT | |
| | | CCCAAATAGGACGAAACGCGC | |
| 86 | RA086 | GCGCGTCCTGGATTCGCGGAAACGCggaataccagcatcg | 1.7 |
| | | tcttgatgcccttggaagtccGCGTACATCCAGCTGACGA | |
| | | GTCCCAAATAGGACGAAACGCGC | |
| 87 | RA087 | GCGCGTCCTGGATTCGCGGAAACGCGTACATCCAGCTGAC | 1.7 |
| | | GAGCGAgaataccagcatcgtcttgatgcccttggaagtc | |
| | | TCGTCCCAAATAGGACGAAACGCGC | |
| 88 | RA088 | GCGCGTCCTGGATTCGCGGAAACGCGTACATCCCGAgaat | 1.7 |
| | | accagcatcgtcttgatgcccttggaagtcTCGAGCTGAC | |
| | | GAGTCCCAAATAGGACGAAACGCGC | |
| 89 | RA089 | GCGCGTCCTGGATTCGCGGAAACGCGTACATCCAGCTGAC | 1.7 |
| | | GAGTCCCAAATAGGACCggaataccagcatcgtcttgatg | |
| | | cccttggaagtccGGAAACGCGC | |
| 90 | RA090 | GCGCGTCCTGGAagtgataccagcatcgtcttgatgccct | 1.7 |
| | | tggcagcactTTCGCGGAAACGCGTACATCCAGCTGACGA | |
| | | GTCCCAAATAGGACGAAACGCGC | |
| 91 | RA091 | ggaataccagcatcgtcttgatgcccttggaagtccGCGC | 1.6 |
| | | GTCCTGGATTCGCGGAAACGCGTACATCCAGCTGACGAGT | |
| | | CCCAAATAGGACGAAACGCGC | |
| 92 | RA092 | GCGCGTCCTGGATTCGCGGAAACGCGTACATCCAGCTGAC | 1.6 |
| | | GAGTCCCAAATAGGACCggaataccagcatcgtcttgatg | |
| | | cccttggaagtccGGCGAAACGCGC | |
| 93 | RA093 | agtgataccagcatcgtcttgatgcccttggcagcactGC | 1.6 |
| | | GCGTCCTGGATTCGCGGAAACGCGTACATCCAGCTGACGA | |
| | | GTCCCAAATAGGACGAAACGCGC | |
| 94 | RA094 | GCGCGTCCTGGATTCGCGGAggaataccagcatcgtcttg | 1.6 |
| | | atgcccttggaagtccAACGCGTACATCCAGCTGACGAGT | |
| | | CCCAAATAGGACGAAACGCGC | |
| 95 | RA095 | GCGCGTCAgaataccagcatcgtcttgatgcccttggaag | 1.6 |
| | | tcTCTGGATTCGCGGAAACGCGTACATCCAGCTGACGAGT | |
| | | CCCAAATAGGACGAAACGCGC | |
| 96 | RA096 | GCGCGTCagtgataccagcatcgtcttgatgcccttggca | 1.6 |
| | | gcactCTGGATTCGCGGAAACGCGTACATCCAGCTGACGA | |
| | | GTCCCAAATAGGACGAAACGCGC | |
| 97 | RA097 | GCGCGTCCTGGATTCGCGGAAACGCGTACATCCAGCTGAC | 1.6 |
| | | GAGTCCCAgaataccagcatcgtcttgatgcccttggaag | |
| | | tcTAAATAGGACGAAACGCGC | |
| 98 | RA098 | GCGCGTCCTGGATTCGCGGAAACGCGTACATCCCCggaat | 1.6 |
| | | accagcatcgtcttgatgcccttggaagtccGGAGCTGAC | |
| | | GAGTCCCAAATAGGACGAAACGCGC | |
| 99 | RA099 | GCGCGTCCTGGATTCGCGGAAACGCGTACATagtgatacc | 1.6 |
| | | agcatcgtcttgatgcccttggcagcactCCAGCTGACGA | |
| | | GTCCCAAATAGGACGAAACGCGC | |
| 100 | RA100 | GCGCGTCCTGGATTCGCGGAAACGCGTACATCCAGCTGAC | 1.6 |
| | | CggaataccagcatcgtcttgatgcccttggaagtccGGA | |
| | | GTCCCAAATAGGACGAAACGCGC | |
| 101 | RA101 | GCGCGTCCTGGATTCGCGGAAACGCGTACATCCAGCTGAC | 1.6 |
| | | GAGTCCCAAATAGGACGAACggaataccagcatcgtcttg | |
| | | atgcccttggaagtccGACGCGC | |
| 102 | RA102 | GCGCGTCggaataccagcatcgtcttgatgcccttggaag | 1.6 |
| | | tccCTGGATTCGCGGAAACGCGTACATCCAGCTGACGAGT | |
| | | CCCAAATAGGACGAAACGCGC | |
| 103 | RA103 | GCGCGTCCTGGATTCGCGGAAACGCGTACATCCAGCTGAC | 1.6 |
| | | ggaataccagcatcgtcttgatgcccttggaagtccGAGT | |
| | | CCCAAATAGGACGAAACGCGC | |
| 104 | RA104 | GCGCGTCCTGGATTCGCGGAAACGCGTACATCCAGCTGAC | 1.6 |
| | | GAGTCCCAAATCGAgaataccagcatcgtcttgatgccct | |
| | | tggaagtcTCGAGGACGAAACGCGC | |
| 105 | RA105 | GCGCGTCCTGGAgaataccagcatcgtcttgatgcccttg | 1.6 |
| | | gaagtcTCGATTCGCGGAAACGCGTACATCCAGCTGACGA | |
| | | GTCCCAAATAGGACGAAACGCGC | |
| 106 | RA106 | GCGCGTCCTGGATTCGCGGAAACGCGTACATCCAGCTGAC | 1.6 |
| | | CggaataccagcatcgtcttgatgcccttggaagtccGGC | |
| | | GAGTCCCAAATAGGACGAAACGCGC | |
| 107 | RA107 | GCGCGTCCTGGATTCGCGGAAACGCGTACATCCCggaata | 1.6 |
| | | ccagcatcgtcttgatgcccttggaagtccGAGCTGACGA | |
| | | GTCCCAAATAGGACGAAACGCGC | |
| 108 | RA108 | GCGCGTCCTGGATTCGCGGCCggaataccagcatcgtctt | 1.6 |
| | | gatgcccttggaagtccGGAAACGCGTACATCCAGCTGAC | |
| | | GAGTCCCAAATAGGACGAAACGCGC | |
| 109 | RA109 | GCGCCGAgaataccagcatcgtcttgatgcccttggaagt | 1.6 |
| | | CTCGGTCCTGGATTCGCGGAAACGCGTACATCCAGCTGAC | |
| | | GAGTCCCAAATAGGACGAAACGCGC | |
| 110 | RA110 | GCGCGTCCTGGggaataccagcatcgtcttgatgcccttg | 1.6 |
| | | gaagtccATTCGCGGAAACGCGTACATCCAGCTGACGAGT | |
| | | CCCAAATAGGACGAAACGCGC | |
| 111 | RA111 | GCGCGTCCTagtgataccagcatcgtcttgatgcccttgg | 1.6 |
| | | cagcactGGATTCGCGGAAACGCGTACATCCAGCTGACGA | |
| | | GTCCCAAATAGGACGAAACGCGC | |
| 112 | RA112 | GCGCGTCCTGGATTCGCGGAAACGCGTACATCCAGggaat | 1.5 |
| | | accagcatcgtcttgatgcccttggaagtccCTGACGAGT | |
| | | CCCAAATAGGACaAAACGCGC | |
| 113 | RA113 | GCGCGTCCTGGATTCGCGGAAACGCGTACATCCAGCTGAC | 1.5 |
| | | GAGTCCCAAATAGGACGAAACGCGC | |
| 114 | RA114 | GCGCGTCCTGGATTCGCGGAAACGCGTACATCCAGCTGAC | 1.5 |
| | | GAGTCCCAAATAGGACGAggaataccagcatcgtcttgat | |
| | | gcccttggaagtccAACGCGC | |
| 115 | RA115 | GCGCGTCCTGGATTCGCGGAAACGCGTACATCCAGCTGAC | 1.5 |
| | | GAGTCCCAAATAGGACGAgaataccagcatcgtcttgatg | |
| | | cccttggaagtcTAAACGCGC | |
| 116 | RA116 | GCGCGTCCTGGATTCGCGGAAACGCGTACATCCAGCTGAC | 1.5 |
| | | GAGTCCCAAATAGGGAgaataccagcatcgtcttgatgcc | |
| | | cttggaagtcTCACGAAACGCGC | |
| 117 | RA117 | GCGCGTCCTGGATCggaataccagcatcgtcttgatgccc | 1.5 |
| | | ttggaagtccGTCGCGGAAACGCGTACATCCAGCTGACGA | |
| | | GTCCCAAATAGGACGAAACGCGC | |
| 118 | RA118 | GCGCGTCCTGGATTCGCGGAAACGCGTACATCCAGCTGAC | 1.5 |
| | | GAGTCCCAAATAGGACGAGAgaataccagcatcgtcttga | |
| | | tgcccttggaagtcTCAACGCGC | |
| 119 | RA119 | GCGCGTCCTGGATTCGCGGAAACGCGTACATCCAGAgaat | 1.5 |
| | | accagcatcgtcttgatgcccttggaagtcTCTGACGAGT | |
| | | CCCAAATAGGACGAAACGCGC | |
| 120 | RA120 | GCGCGTCCCCggaataccagcatcgtcttgatgcccttgg | 1.5 |
| | | aagtccGGTGGATTCGCGGAAACGCGTACATCCAGCTGAC | |
| | | GAGTCCCAAATAGGACGAAACGCGC | |
| 121 | RA121 | GCGCGTCCTGGATTCGAgaataccagcatcgtcttgatgc | 1.5 |
| | | ccttggaagtcTCGCGGAAACGCGTACATCCAGCTGACGA | |
| | | GTCCCAAATAGGACGAAACGCGC | |
| 122 | RA122 | GCGCGTCCTGGATTCGCGGAAACGCGTACATCCAGCTGAC | 1.5 |
| | | GAGTCCCAAATAGGACGAgaataccagcatcgtcttgatg | |
| | | cccttggaagtcTCGAAACGCGC | |
| 123 | RA123 | GCGCGTCCTGGATTCGCGGAAACGCGTCggaataccagca | 1.5 |
| | | tcgtcttgatgcccttggaagtccGACATCCAGCTGACGA | |
| | | GTCCCAAATAGGACGAAACGCGC | |
| 124 | RA124 | GCGCGTCCTGGATTCGCGGAAACGCGTACATCCAGCTGAC | 1.5 |
| | | GCggaataccagcatcgtcttgatgcccttggaagtccGA | |
| | | GTCCCAAATAGGACGAAACGCGC | |
| 125 | RA125 | GCGCGTCCTGGATTCGCGGAAACGCGTACATCCAGCTGAC | 1.5 |
| | | GAGTCCCggaataccagcatcgtcttgatgcccttggaag | |
| | | tccGGCCAAATAGGACGAAACGCGC | |
| 126 | RA126 | GCGCGTCCTGGATTCGCGGAAACGCGTACAAgaataccag | 1.5 |
| | | catcgtcttgatgcccttggaagtcTTCCAGCTGACGAGT | |
| | | CCCAAATAGGACGAAACGCGC | |
| 127 | RA127 | GCGCGTCCTGGATTCGCGGAAACGCGTACATCCAGCTGAC | 1.4 |
| | | GAGTCCCAAATAGGACGAAACGCGCCggaataccagcatc | |
| | | gtcttgatgcccttggaagtccGGC | |
| 128 | RA128 | GCGCGTCCTGGATTCGCGGAAACGCGTAagtgataccagc | 1.4 |
| | | atcgtcttgatgcccttggcagcactCATCCAGCTGACGA | |
| | | GTCCCAAATAGGACGAAACGCGC | |
| 129 | RA129 | GCGCGTCCTGGATTCCACTTCGGGTACATCCAGCTGACGA | 1.4 |
| | | GTCCCAAATAGGACGAAACGCGC | |
| 130 | RA130 | GCGCGTCCTGGATTCGCGGAAACGCGTACATCCAGCTGAg | 1.4 |
| | | aataccagcatcgtcttgatgcccttggaagtcTCGACGA | |
| | | GTCCCAAATAGGACGAAACGCGC | |
| 131 | RA131 | GCGCGTCCTAgaataccagcatcgtcttgatgcccttgga | 1.4 |
| | | agtcTGGATTCGCGGAAACGCGTACATCCAGCTGACGAGT | |
| | | CCCAAATAGGACGAAACGCGC | |
| 132 | RA132 | GCGCGTCCTGGATTCGCggaataccagcatcgtcttgatg | 1.4 |
| | | cccttggaagtccGGAAACGCGTACATCCAGCTGACGAGT | |
| | | CCCAAATAGGACGAAACGCGC | |
| 133 | RA133 | GCGCGAgaataccagcatcgtcttgatgcccttggaagtc | 1.4 |
| | | TCGTCCTGGATTCGCGGAAACGCGTACATCCAGCTGACGA | |
| | | GTCCCAAATAGGACGAAACGCGC | |
| 134 | RA134 | GCGCGTCCTGGATTCGCGGAAACGCGTACATCCAGCTGAC | 1.4 |
| | | GAGTCggaataccagcatcgtcttgatgcccttggaagtc | |
| | | CGCCCAAATAGGACGAAACGCGC | |
| 135 | RA135 | GCGCGTCCTGGATTCGCGGAAACGagtgataccagcatcg | 1.4 |
| | | tcttgatgcccttggcagcactCGTACATCCAGCTGACGA | |
| | | GTCCCAAATAGGACGAAACGCGC | |
| 136 | RA136 | GCGCGTCCTGGATTCGCGGAAACGCGTACATCCAGCTGAC | 1.4 |
| | | GAGTCCCAAATAGGACGAAACGGAgaataccagcatcgtc | |
| | | ttgatgcccttggaagtcTCCGC | |
| 137 | RA137 | GCGCGTCCTGGATTCGCGGAAACGCGTACGAgaataccag | 1.4 |
| | | catcgtcttgatgcccttggaagtcTCATCCAGCTGACGA | |
| | | GTCCCAAATAGGACGAAACGCGC | |
| 138 | RA138 | GCGCggaataccagcatcgtcttgatgcccttggaagtcc | 1.4 |
| | | GCGTCCTGGATTCGCGGAAACGCGTACATCCAGCTGACGA | |
| | | GTCCCAAATAGGACGAAACGCGC | |
| 139 | RA139 | GGAgaataccagcatcgtcttgatgcccttggaagtcTCC | 1.4 |
| | | GCGTCCTGGATTCGCGGAAACGCGTACATCCAGCTGACGA | |
| | | GTCCCAAATAGGACGAAACGCGC | |
| 140 | RA140 | GCGCGTCCTGGATTCGCGGAAACGCGTACATCCAGCTGAC | 1.4 |
| | | GAGTCCggaataccagcatcgtcttgatgcccttggaagt | |
| | | CCGGCCCAAATAGGACGAAACGCGC | |
| 141 | RA141 | GCGCGTCCTGGATTCGCGGAAACGCGTACATCCggaatac | 1.4 |
| | | cagcatcgtcttgatgcccttggaagtccGGCCAGCTGAC | |
| | | GAGTCCCAAATAGGACGAAACGCGC | |
| 142 | RA142 | GCGCGTCCTGGATTCGCGGAAACGCGTACggaataccagc | 1.4 |
| | | atcgtcttgatgcccttggaagtccATCCAGCTGACGAGT | |
| | | CCCAAATAGGACGAAACGCGC | |
| 143 | RA143 | GCGCGTCCTGGATTCGCGGAAACGCGTACATCCAGCTGAC | 1.4 |
| | | GagtgataccagcatcgtcttgatgcccttggcagcactA | |
| | | GTCCCAAATAGGACGAAACGCGC | |
| 144 | RA144 | GCGCGCGAgaataccagcatcgtcttgatgcccttggaag | 1.4 |
| | | tcTCGTCCTGGATTCGCGGAAACGCGTACATCCAGCTGAC | |
| | | GAGTCCCAAATAGGACGAAACGCGC | |
| 145 | RA145 | GCGCggaataccagcatcgtcttgatgcccttggaagtcc | 1.4 |
| | | GTCCTGGATTCGCGGAAACGCGTACATCCAGCTGACGAGT | |
| | | CCCAAATAGGACGAAACGCGC | |
| 146 | RA146 | GCGCGTCCTGGATTCGCGGAAACGCGTACATCCAGCTGAC | 1.4 |
| | | GAGTCCCAAATAggaataccagcatcgtcttgatgccctt | |
| | | ggaagtccGGACGAAACGCGC | |
| 147 | RA147 | GCGCGTCCTGGATTCGCGGAAACGCGTACATCCAGCTGAC | 1.4 |
| | | GAGTAgaataccagcatcgtcttgatgcccttggaagtcT | |
| | | CCCAAATAGGACGAAACGCGC | |
| 148 | RA148 | GCGCGTCCTGGATTCGCGGAAACGCGTACATCCAGCTGAC | 1.4 |
| | | GAGAgaataccagcatcgtcttgatgcccttggaagtcTT | |
| | | CCCAAATAGGACGAAACGCGC | |
| 149 | RA149 | GCGCGGAgaataccagcatcgtcttgatgcccttggaagt | 1.4 |
| | | CTCTCCTGGATTCGCGGAAACGCGTACATCCAGCTGACGA | |
| | | GTCCCAAATAGGACGAAACGCGC | |
| 150 | RA150 | GCGCGTCCTGGATTCGCGGAAACGCGTACATCCAGCTGAC | 1.4 |
| | | GAGTCCCAAATAGGACGAAAggaataccagcatcgtcttg | |
| | | atgcccttggaagtccCGCGC | |
| 151 | RA151 | GCGCGTCCTGGATTCGCGGAAACGCGTACCggaataccag | 1.4 |
| | | catcgtcttgatgcccttggaagtccGGCATCCAGCTGAC | |
| | | GAGTCCCAAATAGGACGAAACGCGC | |
| 152 | RA152 | GCGCGTggaataccagcatcgtcttgatgcccttggaagt | 1.4 |
| | | CCCCTGGATTCGCGGAAACGCGTACATCCAGCTGACGAGT | |
| | | CCCAAATAGGACGAAACGCGC | |
| 153 | RA153 | CGAgaataccagcatcgtcttgatgcccttggaagtcTCG | 1.3 |
| | | GCGCGTCCTGGATTCGCGGAAACGCGTACATCCAGCTGAC | |
| | | GAGTCCCAAATAGGACGAAACGCGC | |
| 154 | RA154 | GCGCGTCCTGGATTCGCGGAAACGCGAgaataccagcatc | 1.3 |
| | | gtcttgatgcccttggaagtcTCGTACATCCAGCTGACGA | |
| | | GTCCCAAATAGGACGAAACGCGC | |
| 155 | RA155 | GCGCGTCCTGGATTCGCGGAAACGCGTACATCCAGCTGAC | 1.3 |
| | | GAGTCCCAAATAGGACGAgaataccagcatcgtcttgatg | |
| | | cccttggaagtcTCGCGAAACGCGC | |
| 156 | RA156 | GCCggaataccagcatcgtcttgatgcccttggaagtccG | 1.3 |
| | | GCGCGTCCTGGATTCGCGGAAACGCGTACATCCAGCTGAC | |
| | | GAGTCCCAAATAGGACGAAACGCGC | |
| 157 | RA157 | GCGCGTCCTGGATTCGCGGAAACGCGTAAgaataccagca | 1.3 |
| | | tcgtcttgatgcccttggaagtcTCATCCAGCTGACGAGT | |
| | | CCCAAATAGGACGAAACGCGC | |
| 158 | RA158 | GCGCGTCCTGGATTCGCGGAAACGCGTACATCCAGCTGAC | 1.3 |
| | | GAGTCCCAAATAGGACGagtgataccagcatcgtcttgat | |
| | | gcccttggcagcactAAACGCGC | |
| 159 | RA159 | GCGCGTCCTGGATTCGCggaataccagcatcgtcttgatg | 1.3 |
| | | cccttggaagtccGCGGAAACGCGTACATCCAGCTGACGA | |
| | | GTCCCAAATAGGACGAAACGCGC | |
| 160 | RA160 | GCGCGTCCTGGATTCGCGGAAACGCGTACATCCAGCTGAC | 1.3 |
| | | GAGggaataccagcatcgtcttgatgcccttggaagtccT | |
| | | CCCAAATAGGACGAAACGCGC | |
| 161 | RA161 | GCGCGTCCTGGATTCGCGGAAACGCGTACATCCAGCTGAC | 1.3 |
| | | GAGTCCCAAATAGGACCCggaataccagcatcgtcttgat | |
| | | gcccttggaagtccGGGAAACGCGC | |
| 162 | RA162 | GCGCGTCCTGGATTCGCGGAAACGCGTACATCCAGCTGAC | 1.3 |
| | | GAGTCCCAAATAGGggaataccagcatcgtcttgatgccc | |
| | | ttggaagtccACGAAACGCGC | |
| 163 | RA163 | GCGCGTCCTGGATTCGCGGAAACGCGTACATCCAGCCgga | 1.3 |
| | | ataccagcatcgtcttgatgcccttggaagtccGGCTGAC | |
| | | GAGTCCCAAATAGGACGAAACGCGC | |
| 164 | RA164 | GCGCGTCCTGGATTCGCGGAAACGCGTACATCCAGCTGAC | 1.3 |
| | | GAGTCCCAAATAGGACGCGAgaataccagcatcgtcttga | |
| | | tgcccttggaagtcTCGAAACGCGC | |
| 165 | RA165 | GCGCGTCCTGGATTCGCGGAAACGCGTAGAgaataccagc | 1.3 |
| | | atcgtcttgatgcccttggaagtcTCCATCCAGCTGACGA | |
| | | GTCCCAAATAGGACGAAACGCGC | |
| 166 | RA166 | GCGCGTCCTGGATTCGCGGAAACGCGTACATCCAGCTGAC | 1.3 |
| | | GAGTCCCACGAgaataccagcatcgtcttgatgcccttgg | |
| | | aagtcTCGAATAGGACGAAACGCGC | |
| 167 | RA167 | GCGCGTCCTGGATTCGCGGAAACGCGTACATCCAGggaat | 1.3 |
| | | accagcatcgtcttgatgcccttggaagtccCTGACGAGT | |
| | | CCCAAATAGGACGAAACGCGC | |
| 168 | RA168 | GCGCGTCCTGGATCGAgaataccagcatcgtcttgatgcc | 1.3 |
| | | cttggaagtcTCGTCGCGGAAACGCGTACATCCAGCTGAC | |
| | | GAGTCCCAAATAGGACGAAACGCGC | |
| 169 | RA169 | GCGCGTCCTGGCCggaataccagcatcgtcttgatgccct | 1.3 |
| | | tggaagtccGGATTCGCGGAAACGCGTACATCCAGCTGAC | |
| | | GAGTCCCAAATAGGACGAAACGCGC | |
| 170 | RA170 | GCGCGTCCTGGATTCGCGGAAACGCGTACATCCAGCTGAC | 1.3 |
| | | GAGTCCCAAATAGGACGAAACGCAgaataccagcatcgtc | |
| | | ttgatgcccttggaagtcTGC | |
| 171 | RA171 | GCGCGTCCTGGATTCGCGGAAACGCGTACACGAgaatacc | 1.3 |
| | | agcatcgtcttgatgcccttggaagtcTCGTCCAGCTGAC | |
| | | GAGTCCCAAATAGGACGAAACGCGC | |
| 172 | RA172 | AgaataccagcatcgtcttgatgcccttggaagtcTGCGC | 1.3 |
| | | GTCCTGGATTCGCGGAAACGCGTACATCCAGCTGACGAGT | |
| | | CCCAAATAGGACGAAACGCGC | |
| 173 | RA173 | GCGCGTCCTGGATTCGCGGAAACGCGTACATCCAGCTGAC | 1.3 |
| | | GAGTCCCAAATAGCggaataccagcatcgtcttgatgccc | |
| | | ttggaagtccGGACGAAACGCGC | |
| 174 | RA174 | GCGCGTCCTGGATTCGCGGAAACGCGTACATCCAGCCgga | 1.3 |
| | | ataccagcatcgtcttgatgcccttggaagtccGTGACGA | |
| | | GTCCCAAATAGGACGAAACGCGC | |
| 175 | RA175 | GCGCGTCCTGGATTCGCGGAAACGCGTACATCCAGCTGAC | 1.3 |
| | | GAGTCCCAAATAGGACGAAACGCGCCGAgaataccagcat | |
| | | cgtcttgatgcccttggaagtcTCG | |
| 176 | RA176 | GCGCGTCCTggaataccagcatcgtcttgatgcccttgga | 1.3 |
| | | agtccGGATTCGCGGAAACGCGTACATCCAGCTGACGAGT | |
| | | CCCAAATAGGACGAAACGCGC | |
| 177 | RA177 | GCGCGTCCTGGATTCGCGGAAACGCGTACATCCAGCTGAC | 1.3 |
| | | GAGTCCCAAATAGGACGAAACGCagtgataccagcatcgt | |
| | | cttgatgcccttggcagcactGC | |
| 178 | RA178 | GCGCGTCCTGGATTCGCGGAAACGCGTACATCCggaatac | 1.3 |
| | | cagcatcgtcttgatgcccttggaagtccAGCTGACGAGT | |
| | | CCCAAATAGGACGAAACGCGC | |
| 179 | RA179 | GCGCGTCCTGGATTCGCGGAAACGCGTACATCCAGCTCgg | 1.3 |
| | | aataccagcatcgtcttgatgcccttggaagtccGGACGA | |
| | | GTCCCAAATAGGACGAAACGCGC | |
| 180 | RA180 | GCGCGTCCTGGATTCGCGGAAACGCGTACATCCAGCTGGA | 1.3 |
| | | gaataccagcatcgtcttgatgcccttggaagtcTCACGA | |
| | | GTCCCAAATAGGACGAAACGCGC | |
| 181 | RA181 | GCGCGCCggaataccagcatcgtcttgatgcccttggaag | 1.3 |
| | | tccGGTCCTGGATTCGCGGAAACGCGTACATCCAGCTGAC | |
| | | GAGTCCCAAATAGGACGAAACGCGC | |
| 182 | RA182 | GCGCGTCCTGGATTCGCGGAAACGCGGAgaataccagcat | 1.3 |
| | | cgtcttgatgcccttggaagtcTCTACATCCAGCTGACGA | |
| | | GTCCCAAATAGGACGAAACGCGC | |
| 183 | RA183 | GCGCGTCCTGGATTCGCGGAAACGCGTACATCCAGCTGAg | 1.3 |
| | | aataccagcatcgtcttgatgcccttggaagtcTACGAGT | |
| | | CCCAAATAGGACGAAACGCGC | |
| 184 | RA184 | GAgaataccagcatcgtcttgatgcccttggaagtcTCGC | 1.3 |
| | | GTCCTGGATTCGCGGAAACGCGTACATCCAGCTGACGAGT | |
| | | CCCAAATAGGACGAAACGCGC | |
| 185 | RA185 | GCGCGTCCTGGATTCGCGGAAACGCGTACATCCAGCTGAC | 1.3 |
| | | GAGTCCCAAATACggaataccagcatcgtcttgatgccct | |
| | | tggaagtccGGGACGAAACGCGC | |
| 186 | RA186 | GCGCGTCCTGGATTCGCGCGAgaataccagcatcgtcttg | 1.3 |
| | | atgcccttggaagtcTCGGAAACGCGTACATCCAGCTGAC | |
| | | GAGTCCCAAATAGGACGAAACGCGC | |
| 187 | RA187 | GCGCGTCCTGGATTCGCGGAAACGCGTACATCCAGCTGAC | 1.3 |
| | | GAGTCCCAAATAGGACGAAACGCGCGAgaataccagcatc | |
| | | gtcttgatgcccttggaagtcTC | |
| 188 | RA188 | GCGCGTCCTGGATTCGCGGAAACGCGTACATCCAGCTGAC | 1.2 |
| | | GAGTCCCAAATAGGACGAAGAgaataccagcatcgtcttg | |
| | | atgcccttggaagtcTCACGCGC | |
| 189 | RA189 | GCGCGTCCTGGATTCggaataccagcatcgtcttgatgcc | 1.2 |
| | | cttggaagtccGCGGAAACGCGTACATCCAGCTGACGAGT | |
| | | CCCAAATAGGACGAAACGCGC | |
| 190 | RA190 | GCGCGTCCTGGATTCGCGagtgataccagcatcgtcttga | 1.2 |
| | | tgcccttggcagcactGAAACGCGTACATCCAGCTGACGA | |
| | | GTCCCAAATAGGACGAAACGCGC | |
| 191 | RA191 | GCGCGTCCTGGATTCGCGGAACGAgaataccagcatcgtc | 1.2 |
| | | ttgatgcccttggaagtcTCGACGCGTACATCCAGCTGAC | |
| | | GAGTCCCAAATAGGACGAAACGCGC | |
| 192 | RA192 | GCGCGTCCTGGATTCGCGGAAACGCGTACATCCAGCTGAC | 1.2 |
| | | GAGTCCCAAATggaataccagcatcgtcttgatgcccttg | |
| | | gaagtccAGGACGAAACGCGC | |
| 193 | RA193 | CCggaataccagcatcgtcttgatgcccttggaagtccGG | 1.2 |
| | | GCGCGTCCTGGATTCGCGGAAACGCGTACATCCAGCTGAC | |
| | | GAGTCCCAAATAGGACGAAACGCGC | |
| 194 | RA194 | GCGCGTCCTGGATTCGCGGAAACGCGTACATCCAGCTGAC | 1.2 |
| | | GAgaataccagcatcgtcttgatgcccttggaagtcTCGA | |
| | | GTCCCAAATAGGACGAAACGCGC | |
| 195 | RA195 | GCGCGTCGAgaataccagcatcgtcttgatgcccttggaa | 1.2 |
| | | gtcTCGCCTGGATTCGCGGAAACGCGTACATCCAGCTGAC | |
| | | GAGTCCCAAATAGGACGAAACGCGC | |
| 196 | RA196 | GCGCGTCCTGGATTCGCGGAAACGCGTACATCCAGCTGAC | 1.2 |
| | | GAGTCCCAAATGAgaataccagcatcgtcttgatgccctt | |
| | | ggaagtcTCAGGACGAAACGCGC | |
| 197 | RA197 | GCGCGTCCTGGATTCGCGGAAACGCGTACATCCAGCTGAC | 1.2 |
| | | GAGTCCCAAATAGGACaAAACGCGC | |
| 198 | RA198 | GCGCGTCCTGAgaataccagcatcgtcttgatgcccttgg | 1.2 |
| | | aagtcTGATTCGCGGAAACGCGTACATCCAGCTGACGAGT | |
| | | CCCAAATAGGACGAAACGCGC | |
| 199 | RA199 | GCGCGTCCTGGATTCGCGGAAACagtgataccagcatcgt | 1.2 |
| | | cttgatgcccttggcagcactGCGTACATCCAGCTGACGA | |
| | | GTCCCAAATAGGACGAAACGCGC | |
| 200 | RA200 | GCGCGTCCTGGATTCGCGGAAACGCGTACATCCGAgaata | 1.2 |
| | | ccagcatcgtcttgatgcccttggaagtcTCGCAGCTGAC | |
| | | GAGTCCCAAATAGGACGAAACGCGC | |
| 201 | RA201 | GCGCGTCCTGGATTCGCGGAAACGCGAgaataccagcatc | 1.2 |
| | | gtcttgatgcccttggaagtcTCGCGTACATCCAGCTGAC | |
| | | GAGTCCCAAATAGGACGAAACGCGC | |
| 202 | RA202 | GCGCGTCCTGGATagtgataccagcatcgtcttgatgccc | 1.2 |
| | | ttggcagcactTCGCGGAAACGCGTACATCCAGCTGACGA | |
| | | GTCCCAAATAGGACGAAACGCGC | |
| 203 | RA203 | GCGCGTCCTGGATTCGCGGAAACGCGTACATCCAGCTGAC | 1.2 |
| | | GAGTCCCAAATAGGAAgaataccagcatcgtcttgatgcc | |
| | | cttggaagtcTCGAAACGCGC | |
| 204 | RA204 | GCGCGTCCCggaataccagcatcgtcttgatgcccttgga | 1.2 |
| | | agtccGTGGATTCGCGGAAACGCGTACATCCAGCTGACGA | |
| | | GTCCCAAATAGGACGAAACGCGC | |
| 205 | RA205 | GCGCGTCCTGGATTCGCGGAAACGCGTACATCCAGCTGAC | 1.2 |
| | | GAGTCCCAAATAGGACGAAACGCggaataccagcatcgtc | |
| | | ttgatgcccttggaagtccGC | |
| 206 | RA206 | GCGCGTCCTGGATTCGCCggaataccagcatcgtcttgat | 1.2 |
| | | gcccttggaagtccGGCGGAAACGCGTACATCCAGCTGAC | |
| | | GAGTCCCAAATAGGACGAAACGCGC | |
| 207 | RA207 | GCGCGTCCTGGATTCGCGGAAACGCGTACATCCAGCTGAC | 1.2 |
| | | GAGTCCCAAGAgaataccagcatcgtcttgatgcccttgg | |
| | | aagtcTCATAGGACGAAACGCGC | |
| 208 | RA208 | GCGCGTCCTGGATTCGCGGAAACGCGTACATCCAGCTGAC | 1.2 |
| | | GAGTCCCAAACCggaataccagcatcgtcttgatgccctt | |
| | | ggaagtccGGTAGGACGAAACGCGC | |
| 209 | RA209 | GCGCGTCCTGGATTCGCGGAGAgaataccagcatcgtctt | 1.2 |
| | | gatgcccttggaagtcTCAACGCGTACATCCAGCTGACGA | |
| | | GTCCCAAATAGGACGAAACGCGC | |
| 210 | RA210 | GCTGTCACCGGAATACCAGCATCGTCTTGATGCCCTTGGA | 1.2 |
| | | AGTCCGGTCTGATGAGTCCAAAAAGGACGAAACAGC | |
| 211 | RA211 | GCGCGTCCTGGATTCGggaataccagcatcgtcttgatgc | 1.2 |
| | | ccttggaagtccCGGAAACGCGTACATCCAGCTGACGAGT | |
| | | CCCAAATAGGACGAAACGCGC | |
| 212 | RA212 | GCGCGTCCTGGATTCAgaataccagcatcgtcttgatgcc | 1.2 |
| | | cttggaagtcTGCGGAAACGCGTACATCCAGCTGACGAGT | |
| | | CCCAAATAGGACGAAACGCGC | |
| 213 | RA213 | GCGCGTCCTGGATTCGCGGAAACGCGTACATCCAGCTCGA | 1.2 |
| | | gaataccagcatcgtcttgatgcccttggaagtcTCGGAC | |
| | | GAGTCCCAAATAGGACGAAACGCGC | |
| 214 | RA214 | GCGCGTCCTGGATTCGCGAgaataccagcatcgtcttgat | 1.2 |
| | | gcccttggaagtcTCGGAAACGCGTACATCCAGCTGACGA | |
| | | GTCCCAAATAGGACGAAACGCGC | |
| 215 | RA215 | GCTGTCACCGGATGTGCTTTCCGGTACGTGAGGTCCGTGA | 1.2 |
| | | GGACGAAACAGC | |
| 216 | RA216 | GCGCGTCCTGGATTCGCGGAAACGCGTACATCCAGCTGAC | 1.2 |
| | | GAGTCagtgataccagcatcgtcttgatgcccttggcagc | |
| | | actCCAAATAGGACGAAACGCGC | |
| 217 | RA217 | GCGCGTCCTGGATTCCGAgaataccagcatcgtcttgatg | 1.2 |
| | | cccttggaagtcTCGGCGGAAACGCGTACATCCAGCTGAC | |
| | | GAGTCCCAAATAGGACGAAACGCGC | |
| 218 | RA218 | GCGCGTCCTGGATTCGCGGAAACGCGTACATCCAagtgat | 1.2 |
| | | accagcatcgtcttgatgcccttggcagcactGCTGACGA | |
| | | GTCCCAAATAGGACGAAACGCGC | |
| 219 | RA219 | GCGCGTCCTGGATTCGCGGAAACGCGTACATCCAGCggaa | 1.2 |
| | | taccagcatcgtcttgatgcccttggaagtccTGACGAGT | |
| | | CCCAAATAGGACGAAACGCGC | |
| 220 | RA220 | GCCGAgaataccagcatcgtcttgatgcccttggaagtcT | 1.2 |
| | | CGGCGTCCTGGATTCGCGGAAACGCGTACATCCAGCTGAC | |
| | | GAGTCCCAAATAGGACGAAACGCGC | |
| 221 | RA221 | GCGCGTCCTGGATTCGCGGAAACGCGTACATCCAGCTGAC | 1.2 |
| | | ggaataccagcatcgtcttgatgcccttggaagtccGCGA | |
| | | GTCCCAAATAGGACGAAACGCGC | |
| 222 | RA222 | GCGCGTCCTGGATTCGCGGAAACGCGTACATCCAGCTGAC | 1.2 |
| | | AgaataccagcatcgtcttgatgcccttggaagtcTGAGT | |
| | | CCCAAATAGGACGAAACGCGC | |
| 223 | RA223 | GCGCGTCCTGCGAgaataccagcatcgtcttgatgccctt | 1.2 |
| | | ggaagtcTCGGATTCGCGGAAACGCGTACATCCAGCTGAC | |
| | | GAGTCCCAAATAGGACGAAACGCGC | |
| 224 | RA224 | GCGCGTCCTGGATTCGCGGAAACGCGTACATCCAGCTGAC | 1.1 |
| | | GAGTCCCAAATAGAgaataccagcatcgtcttgatgccct | |
| | | tggaagtcTGACGAAACGCGC | |
| 225 | RA225 | GCGCGTCCTGGATTCGCGGAAACGCGTACATCCAGCTGAC | 1.1 |
| | | GAGTCCCAAATAAgaataccagcatcgtcttgatgccctt | |
| | | ggaagtcTGGACGAAACGCGC | |
| 226 | RA226 | GCGCGTCCTGGATTCGCGGAAACGCGTACATCCAGCTGAa | 1.1 |
| | | gtgataccagcatcgtcttgatgcccttggcagcactCGA | |
| | | GTCCCAAATAGGACGAAACGCGC | |
| 227 | RA227 | GCGCGTCCGAgaataccagcatcgtcttgatgcccttgga | 1.1 |
| | | agtcTCGCTGGATTCGCGGAAACGCGTACATCCAGCTGAC | |
| | | GAGTCCCAAATAGGACGAAACGCGC | |
| 228 | RA228 | GCGCGTCCTGGATTCGCGGAAACGCGTACATCCAGCTGgg | 1.1 |
| | | aataccagcatcgtcttgatgcccttggaagtccACGAGT | |
| | | CCCAAATAGGACGAAACGCGC | |
| 229 | RA229 | GCGCCggaataccagcatcgtcttgatgcccttggaagtc | 1.1 |
| | | CGGCGTCCTGGATTCGCGGAAACGCGTACATCCAGCTGAC | |
| | | GAGTCCCAAATAGGACGAAACGCGC | |
| 230 | RA230 | GCGCGTCCTGAgaataccagcatcgtcttgatgcccttgg | 1.1 |
| | | aagtcTCGGATTCGCGGAAACGCGTACATCCAGCTGACGA | |
| | | GTCCCAAATAGGACGAAACGCGC | |
| 231 | RA231 | GCGAgaataccagcatcgtcttgatgcccttggaagtcTC | 1.1 |
| | | GCGTCCTGGATTCGCGGAAACGCGTACATCCAGCTGACGA | |
| | | GTCCCAAATAGGACGAAACGCGC | |
| 232 | RA232 | GCCCggaataccagcatcgtcttgatgcccttggaagtcc | 1.1 |
| | | GGGCGTCCTGGATTCGCGGAAACGCGTACATCCAGCTGAC | |
| | | GAGTCCCAAATAGGACGAAACGCGC | |
| 233 | RA233 | GCagtgataccagcatcgtcttgatgcccttggcagcact | 1.1 |
| | | GCGTCCTGGATTCGCGGAAACGCGTACATCCAGCTGACGA | |
| | | GTCCCAAATAGGACGAAACGCGC | |
| 234 | RA234 | GCGCGTCCTGGATTCGCGGAAACGCGTACATCCAGCTGAC | 1.1 |
| | | GAGTCCCAAATAGGACGAAACCggaataccagcatcgtct | |
| | | tgatgcccttggaagtccGGCGC | |
| 235 | RA235 | GCGCGTCCTGGATTCGCGGAAACGCGTACATCCAGCTGAC | 1.1 |
| | | GAGTCCCAAATAGGAGAgaataccagcatcgtcttgatgc | |
| | | ccttggaagtcTCCGAAACGCGC | |
| 236 | RA236 | GCGCGTCCTGGATTCGCGGAAACGCGTACACCggaatacc | 1.1 |
| | | agcatcgtcttgatgcccttggaagtccGGTCCAGCTGAC | |
| | | GAGTCCCAAATAGGACGAAACGCGC | |
| 237 | RA237 | GCGCGTCCTGGATTCGCGGAAACGCGTACATCCAGCTGAC | 1.1 |
| | | GGAgaataccagcatcgtcttgatgcccttggaagtcTCA | |
| | | GTCCCAAATAGGACGAAACGCGC | |
| 238 | RA238 | GCGCGTCCTGGATTAgaataccagcatcgtcttgatgccc | 1.1 |
| | | ttggaagtcTCGCGGAAACGCGTACATCCAGCTGACGAGT | |
| | | CCCAAATAGGACGAAACGCGC | |
| 239 | RA239 | GCGCGTCCTGGATTCGCGGAAACGCGTACATCCAGCTGAC | 1.1 |
| | | CCggaataccagcatcgtcttgatgcccttggaagtccGG | |
| | | GAGTCCCAAATAGGACGAAACGCGC | |
| 240 | RA240 | GCGCGTCCTGGATTCGCGGAAACGCGTACATCCAGCTGAC | 1.1 |
| | | GAGTCCCCGAgaataccagcatcgtcttgatgcccttgga | |
| | | agtcTCGAAATAGGACGAAACGCGC | |
| 241 | RA241 | GCGCGTCCTCCggaataccagcatcgtcttgatgcccttg | 1.1 |
| | | gaagtccGGGGATTCGCGGAAACGCGTACATCCAGCTGAC | |
| | | GAGTCCCAAATAGGACGAAACGCGC | |
| 242 | RA242 | GCGCGTCCTGGATTCCggaataccagcatcgtcttgatgc | 1.1 |
| | | ccttggaagtccGGCGCGGAAACGCGTACATCCAGCTGAC | |
| | | GAGTCCCAAATAGGACGAAACGCGC | |
| 243 | RA243 | GCGCGTCCTGGATTCGCGGAAACGCGTACATCCAGCCCgg | 1.1 |
| | | aataccagcatcgtcttgatgcccttggaagtccGGTGAC | |
| | | GAGTCCCAAATAGGACGAAACGCGC | |
| 244 | RA244 | GCGCGTCCTGGATTCGCGGAAACGCGTCCggaataccagc | 1.1 |
| | | atcgtcttgatgcccttggaagtccGGACATCCAGCTGAC | |
| | | GAGTCCCAAATAGGACGAAACGCGC | |
| 245 | RA245 | GggaataccagcatcgtcttgatgcccttggaagtccCGC | 1.1 |
| | | GTCCTGGATTCGCGGAAACGCGTACATCCAGCTGACGAGT | |
| | | CCCAAATAGGACGAAACGCGC | |
| 246 | RA246 | GCGCGTCCTGGATTCGCGGAgaataccagcatcgtcttga | 1.1 |
| | | tgcccttggaagtcTCGAAACGCGTACATCCAGCTGACGA | |
| | | GTCCCAAATAGGACGAAACGCGC | |
| 247 | RA247 | GCGCGTCCTGGATTCGCGGAAACGCGTACATCCAGCTGAg | 1.1 |
| | | gaataccagcatcgtcttgatgcccttggaagtccCGAGT | |
| | | CCCAAATAGGACGAAACGCGC | |
| 248 | RA248 | GCGCGTCCTGGATTCGCGGAAACGCGTACATCCAGCTGAC | 1.1 |
| | | GAGTCCagtgataccagcatcgtcttgatgcccttggcag | |
| | | cactCAAATAGGACGAAACGCGC | |
| 249 | RA249 | GCGCGTCCTGGATTCGCGGAAACGCGTACATCCACCggaa | 1.1 |
| | | taccagcatcgtcttgatgcccttggaagtccGGGCTGAC | |
| | | GAGTCCCAAATAGGACGAAACGCGC | |
| 250 | RA250 | GCGCGagtgataccagcatcgtcttgatgcccttggcagc | 1.1 |
| | | actTCCTGGATTCGCGGAAACGCGTACATCCAGCTGACGA | |
| | | GTCCCAAATAGGACGAAACGCGC | |
| 251 | RA251 | GCGCGTCCTGGACCggaataccagcatcgtcttgatgccc | 1.1 |
| | | ttggaagtccGGTTCGCGGAAACGCGTACATCCAGCTGAC | |
| | | GAGTCCCAAATAGGACGAAACGCGC | |
| 252 | RA252 | GCGCGTCCTGGATTCGCGGAagtgataccagcatcgtctt | 1.1 |
| | | gatgcccttggcagcactAACGCGTACATCCAGCTGACGA | |
| | | GTCCCAAATAGGACGAAACGCGC | |
| 253 | RA253 | GCGCGTCCTGGATTCGCGGAAACGCGTACATggaatacca | 1.1 |
| | | gcatcgtcttgatgcccttggaagtccCCAGCTGACGAGT | |
| | | CCCAAATAGGACGAAACGCGC | |
| 254 | RA254 | GCGCGTCCTGGATTCGCGGAAACGCGTACATCCAGCAgaa | 1.1 |
| | | taccagcatcgtcttgatgcccttggaagtcTTGACGAGT | |
| | | CCCAAATAGGACGAAACGCGC | |
| 255 | RA255 | GCGCGTCCTGGATTCGCGGAAACGCGTACATCCAGCTGAC | 1.1 |
| | | GAGTCCCAAATAGGACGAAACCggaataccagcatcgtct | |
| | | tgatgcccttggaagtccGGCGCGC | |
| 256 | RA256 | GCGCGTCCTGGATTCGCGGAAACGCGTACATGAgaatacc | 1.1 |
| | | agcatcgtcttgatgcccttggaagtcTCCCAGCTGACGA | |
| | | GTCCCAAATAGGACGAAACGCGC | |
| 257 | RA257 | GCGCGTCCTGGATTCGCGGAAACGCCggaataccagcatc | 1.1 |
| | | gtcttgatgcccttggaagtccGGTACATCCAGCTGACGA | |
| | | GTCCCAAATAGGACGAAACGCGC | |
| 258 | RA258 | GCGCGTCCTGGATTagtgataccagcatcgtcttgatgcc | 1.1 |
| | | cttggcagcactCGCGGAAACGCGTACATCCAGCTGACGA | |
| | | GTCCCAAATAGGACGAAACGCGC | |
| 259 | RA259 | GCGCGTCCTGGATTCGCGGAAACGCGTACATCCAGCTGAC | 1.1 |
| | | GAGTCCCAAATAGGACGAAAagtgataccagcatcgtctt | |
| | | gatgcccttggcagcactCGCGC | |
| 260 | RA260 | GCGCGTCCTGGATTCGCGGAAACGCCggaataccagcatc | 1.1 |
| | | gtcttgatgcccttggaagtccGGCGTACATCCAGCTGAC | |
| | | GAGTCCCAAATAGGACGAAACGCGC | |
| 261 | RA261 | GCGCGTCCTGGATTCGCGGAAACGCGTACATCCAGCTGAC | 1.1 |
| | | GAGTCCCAACGAgaataccagcatcgtcttgatgcccttg | |
| | | gaagtcTCGATAGGACGAAACGCGC | |
| 262 | RA262 | GCTGTCACCGGAATACCAGCATCGTCTTGATGCCCTTGGA | 1.1 |
| | | AGTCCGGTCTGATGAGTCCCATAAGGACGAAACAGC | |
| 263 | RA263 | GCGCGTCCTGGATTCGCGGAAACGCGTACATCCAGCTGAC | 1.1 |
| | | GAGTCCCAAAagtgataccagcatcgtcttgatgcccttg | |
| | | gcagcactTAGGACGAAACGCGC | |
| 264 | RA264 | GCGCGTCCTGGATTCGCGGAAACGCGTACATCCAGCTAga | 1.0 |
| | | ataccagcatcgtcttgatgcccttggaagtcTGACGAGT | |
| | | CCCAAATAGGACGAAACGCGC | |
| 265 | RA265 | GCGCGTCCTGGATTCGCGGAAACGCGTACATCCAGCTGAC | 1.0 |
| | | GAGTCCCAAAggaataccagcatcgtcttgatgcccttgg | |
| | | aagtccTAGGACGAAACGCGC | |
| 266 | RA266 | GCGCGTCCTGGATTCGCGGAAACGCGCGAgaataccagca | 1.0 |
| | | tcgtcttgatgcccttggaagtcTCGTACATCCAGCTGAC | |
| | | GAGTCCCAAATAGGACGAAACGCGC | |
| 267 | RA267 | GCGCGTCCTGGATTCGCGGAAACGCGTACATCCAGCTGAC | 1.0 |
| | | GAGTggaataccagcatcgtcttgatgcccttggaagtcc | |
| | | CCCAAATAGGACGAAACGCGC | |
| 268 | RA268 | GCGCGTCCTGGATTCGCGGAAACGCGTACATCCAGCTGAC | 1.0 |
| | | GAGTCCCAAATAGGACGAAACGCGGAgaataccagcatcg | |
| | | tcttgatgcccttggaagtcTCC | |
| 269 | RA269 | GCGCGTCCTGGATTCGCGGAAACGCGAgaataccagcatc | 1.0 |
| | | gtcttgatgcccttggaagtcTTACATCCAGCTGACGAGT | |
| | | CCCAAATAGGACGAAACGCGC | |
| 270 | RA270 | GCGCGTCCTGGATTCGCGGAAACGCGTACATCCAGCTGAC | 1.0 |
| | | GAGTCCCAAATAGGACGAAgaataccagcatcgtcttgat | |
| | | gcccttggaagtcTAACGCGC | |
| 271 | RA271 | GCGCGTCCTGGATTCGCGGAAACGCGTACATCCAGCTGAC | 1.0 |
| | | GAGTCCCAACCggaataccagcatcgtcttgatgcccttg | |
| | | gaagtccGGATAGGACGAAACGCGC | |
| 272 | RA272 | GCGCGTCCTGGATTCGCGGAAACGCGTACATCCAGCTGAC | 1.0 |
| | | GAGTCCCAAATAGGACGAAACGCGCAgaataccagcatcg | |
| | | tcttgatgcccttggaagtcT | |
| 273 | RA273 | GCGCGTCCTGGATTCGCGggaataccagcatcgtcttgat | 1.0 |
| | | gcccttggaagtccGAAACGCGTACATCCAGCTGACGAGT | |
| | | CCCAAATAGGACGAAACGCGC | |
| 274 | RA274 | GCGCGTCCTGGATTCGCGGAAACGCGTACATCCAGCTGAC | 1.0 |
| | | GAGTCCCAAAGAgaataccagcatcgtcttgatgcccttg | |
| | | gaagtcTCTAGGACGAAACGCGC | |
| 275 | RA275 | GCGCGTCCTGGATTGAgaataccagcatcgtcttgatgcc | 1.0 |
| | | cttggaagtcTCCGCGGAAACGCGTACATCCAGCTGACGA | |
| | | GTCCCAAATAGGACGAAACGCGC | |
| 276 | RA276 | GCGCGTCCTGGATTCGCGGAAACGCGTACATCCAGCTGAC | 1.0 |
| | | GAAgaataccagcatcgtcttgatgcccttggaagtcTGT | |
| | | CCCAAATAGGACGAAACGCGC | |
| 277 | RA277 | GCGCGTCCTGGATTCGCGGAAACGCGTACATCCGAgaata | 1.0 |
| | | ccagcatcgtcttgatgcccttggaagtcTCAGCTGACGA | |
| | | GTCCCAAATAGGACGAAACGCGC | |
| 278 | RA278 | GCGCGTCCTGGATTCGagtgataccagcatcgtcttgatg | 1.0 |
| | | cccttggcagcactCGGAAACGCGTACATCCAGCTGACGA | |
| | | GTCCCAAATAGGACGAAACGCGC | |
| 279 | RA279 | GCGCGTCCTGGATTCGCGGAAACGCGTACATCCAGCTGAC | 1.0 |
| | | GAGTCCCAAATAGGACGACGAgaataccagcatcgtcttg | |
| | | atgcccttggaagtcTCGAACGCGC | |
| 280 | RA280 | GCGCGTCCTGGATTCGCGGAAACGCGTACATCCAGCTGAC | 1.0 |
| | | GAGTCCCAAATAGGAggaataccagcatcgtcttgatgcc | |
| | | cttggaagtccCGAAACGCGC | |
| 281 | RA281 | GCGCGTCCTGGATTCGCGGAAACGCGTACATCCAGCTGAC | 1.0 |
| | | GAGTCCCAAATACGAgaataccagcatcgtcttgatgccc | |
| | | ttggaagtcTCGGGACGAAACGCGC | |
| 282 | RA282 | GCGCGTCCTGGATTCGCGAgaataccagcatcgtcttgat | 1.0 |
| | | gcccttggaagtcTCGCGGAAACGCGTACATCCAGCTGAC | |
| | | GAGTCCCAAATAGGACGAAACGCGC | |
| 283 | RA283 | GCGCGTCCTGGAAgaataccagcatcgtcttgatgccctt | 1.0 |
| | | ggaagtcTTTCGCGGAAACGCGTACATCCAGCTGACGAGT | |
| | | CCCAAATAGGACGAAACGCGC | |
| 284 | RA284 | GCGCGTCCTGGATTCGCGGAAACggaataccagcatcgtc | 1.0 |
| | | ttgatgcccttggaagtccGCGTACATCCAGCTGACGAGT | |
| | | CCCAAATAGGACGAAACGCGC | |
| 285 | RA285 | GCGCGTCCTGGATTCGCGGAAACGCagtgataccagcatc | 1.0 |
| | | gtcttgatgcccttggcagcactGTACATCCAGCTGACGA | |
| | | GTCCCAAATAGGACGAAACGCGC | |
| 286 | RA286 | GCGCGTCCTGGATTCGCGGAAAAgaataccagcatcgtct | 1.0 |
| | | tgatgcccttggaagtcTCGCGTACATCCAGCTGACGAGT | |
| | | CCCAAATAGGACGAAACGCGC | |
| 287 | RA287 | GCGCGTCCTGGATTCGCGGAAACGCGTACATCCAGCTGAC | 1.0 |
| | | GAagtgataccagcatcgtcttgatgcccttggcagcact | |
| | | GTCCCAAATAGGACGAAACGCGC | |
| 288 | RA288 | GCGCGTCCTGGATTCGCGGAAACGCGTACATCCAGCTGAC | 1.0 |
| | | GAGTCCCAAATAagtgataccagcatcgtcttgatgccct | |
| | | tggcagcactGGACGAAACGCGC | |
| 289 | RA289 | GCGCGTCCTGGATTCGCGGAAACGCGTACATCCAggaata | 1.0 |
| | | ccagcatcgtcttgatgcccttggaagtccGCTGACGAGT | |
| | | CCCAAATAGGACGAAACGCGC | |
| 290 | RA290 | GCGCGTCCTGGATTCGCGGAAACGCGTACATCCAGCTGAC | 1.0 |
| | | GACCggaataccagcatcgtcttgatgcccttggaagtcc | |
| | | GGGTCCCAAATAGGACGAAACGCGC | |
| 291 | RA291 | GCTGTCACCggaataccagcatcgtcttgatgcccttgga | 1.0 |
| | | agtccGGTCTGATGAGTCCCATAAGGACGAAACAGC | |
| 292 | RA292 | GCGCGTCggaataccagcatcgtcttgatgcccttggaag | 1.0 |
| | | tccGCCTGGATTCGCGGAAACGCGTACATCCAGCTGACGA | |
| | | GTCCCAAATAGGACGAAACGCGC | |
| 293 | RA293 | GCGggaataccagcatcgtcttgatgcccttggaagtccC | 1.0 |
| | | GTCCTGGATTCGCGGAAACGCGTACATCCAGCTGACGAGT | |
| | | CCCAAATAGGACGAAACGCGC | |
| 294 | RA294 | GCGCGTCCTGGATTCGCGGAAACGAgaataccagcatcgt | 1.0 |
| | | cttgatgcccttggaagtcTCGCGTACATCCAGCTGACGA | |
| | | GTCCCAAATAGGACGAAACGCGC | |
| 295 | RA295 | GCGCGTCCTGGATTCGCGGAAAggaataccagcatcgtct | 1.0 |
| | | tgatgcccttggaagtccCGCGTACATCCAGCTGACGAGT | |
| | | CCCAAATAGGACGAAACGCGC | |
| 296 | RA296 | GCGCGTCCTGGATTCGCGGAAACGCGTACATCCAGCTGAC | 1.0 |
| | | GAGTCCCAAATAGGAgaataccagcatcgtcttgatgccc | |
| | | ttggaagtcTACGAAACGCGC | |
| 297 | RA297 | GCGCGggaataccagcatcgtcttgatgcccttggaagtc | 1.0 |
| | | CTCCTGGATTCGCGGAAACGCGTACATCCAGCTGACGAGT | |
| | | CCCAAATAGGACGAAACGCGC | |
| 298 | RA298 | GCCggaataccagcatcgtcttgatgcccttggaagtccG | 1.0 |
| | | GCGTCCTGGATTCGCGGAAACGCGTACATCCAGCTGACGA | |
| | | GTCCCAAATAGGACGAAACGCGC | |
| 299 | RA299 | GCGCGTagtgataccagcatcgtcttgatgcccttggcag | 1.0 |
| | | cactCCTGGATTCGCGGAAACGCGTACATCCAGCTGACGA | |
| | | GTCCCAAATAGGACGAAACGCGC | |
| 300 | RA300 | GCGCGTCCTGGCGAgaataccagcatcgtcttgatgccct | 1.0 |
| | | tggaagtcTCGATTCGCGGAAACGCGTACATCCAGCTGAC | |
| | | GAGTCCCAAATAGGACGAAACGCGC | |
| 301 | RA301 | GCGagtgataccagcatcgtcttgatgcccttggcagcac | 1.0 |
| | | tCGTCCTGGATTCGCGGAAACGCGTACATCCAGCTGACGA | |
| | | GTCCCAAATAGGACGAAACGCGC | |
| 302 | RA302 | GCGCGTCCTGGATTCGCGGAAACGCGTACATCCAGCTGAC | 1.0 |
| | | GAgaataccagcatcgtcttgatgcccttggaagtcTAGT | |
| | | CCCAAATAGGACGAAACGCGC | |
| 303 | RA303 | GCGCCggaataccagcatcgtcttgatgcccttggaagtc | 1.0 |
| | | CGGTCCTGGATTCGCGGAAACGCGTACATCCAGCTGACGA | |
| | | GTCCCAAATAGGACGAAACGCGC | |
| 304 | RA304 | GCGCGAgaataccagcatcgtcttgatgcccttggaagtc | 1.0 |
| | | TCGCGTCCTGGATTCGCGGAAACGCGTACATCCAGCTGAC | |
| | | GAGTCCCAAATAGGACGAAACGCGC | |
| 305 | RA305 | GCGCGTCCTGGATTCGCGGAAACGCGTACATCagtgatac | 1.0 |
| | | cagcatcgtcttgatgcccttggcagcactCAGCTGACGA | |
| | | GTCCCAAATAGGACGAAACGCGC | |
| 306 | RA306 | GCGCGggaataccagcatcgtcttgatgcccttggaagtc | 1.0 |
| | | CTCCTGGATTCGCGGAAACGCGTACATCCAGCTGACGAGT | |
| | | CCCAAATAGGACaAAACGCGC | |
| 307 | RA307 | GCGCGTCCTGGATTCGCGGAAACGCGTACATCCAGCTGAC | 1.0 |
| | | GAGTGAgaataccagcatcgtcttgatgcccttggaagtc | |
| | | TCCCCAAATAGGACGAAACGCGC | |
| 308 | RA308 | GCGCGTCCTGGATTCGCGGAAACGCGTACATCCAGCagtg | 1.0 |
| | | ataccagcatcgtcttgatgcccttggcagcactTGACGA | |
| | | GTCCCAAATAGGACGAAACGCGC | |
| 309 | RA309 | GCGCGAgaataccagcatcgtcttgatgcccttggaagtc | 1.0 |
| | | TTCCTGGATTCGCGGAAACGCGTACATCCAGCTGACGAGT | |
| | | CCCAAATAGGACGAAACGCGC | |
| 310 | RA310 | GCGCGTCCTGGATTCGCGGAAACGCGTACATCCAGCTGAC | 1.0 |
| | | GAGTCCCAAATAgaataccagcatcgtcttgatgcccttg | |
| | | gaagtcTAGGACGAAACGCGC | |
| 311 | RA311 | GCGCGTCCTGGATTCGCGGAAACGCGTCGAgaataccagc | 1.0 |
| | | atcgtcttgatgcccttggaagtcTCGACATCCAGCTGAC | |
| | | GAGTCCCAAATAGGACGAAACGCGC | |
| 312 | RA312 | GCGCGTCCTGGATTCGCGGAAACGCGTACATCCAGCTGAC | 1.0 |
| | | GAggaataccagcatcgtcttgatgcccttggaagtccGT | |
| | | CCCAAATAGGACGAAACGCGC | |
| 313 | RA313 | GCGCGTCCTGGATTCGCGGAAACGCGTACATCCAGCTGAC | 1.0 |
| | | GAGTCCCAACggaataccagcatcgtcttgatgcccttgg | |
| | | aagtccGATAGGACGAAACGCGC | |
| 314 | RA314 | GCGCGTCCTGGATTCGCGGAAACGCGTACATCCAGCTGAC | 1.0 |
| | | GAGTCCCAAATAGggaataccagcatcgtcttgatgccct | |
| | | tggaagtccGACGAAACGCGC | |
| 315 | RA315 | GCGCGTCCTGGATTCGCGGAAACGCGTACATCCAGCTGAC | 1.0 |
| | | GAGTCCCAAATAGGACGAACGAgaataccagcatcgtctt | |
| | | gatgcccttggaagtcTCGACGCGC | |
| 316 | RA316 | GCGCGTCCTGGATggaataccagcatcgtcttgatgccct | 0.9 |
| | | tggaagtccTCGCGGAAACGCGTACATCCAGCTGACGAGT | |
| | | CCCAAATAGGACaAAACGCGC | |
| 317 | RA317 | GCGCGTCCTGGATTCGCGGAAACGCGTACATCCAGCTGAC | 0.9 |
| | | GAGTCCCAAATAGCCggaataccagcatcgtcttgatgcc | |
| | | cttggaagtccGGGACGAAACGCGC | |
| 318 | RA318 | GCggaataccagcatcgtcttgatgcccttggaagtccGC | 0.9 |
| | | GCGTCCTGGATTCGCGGAAACGCGTACATCCAGCTGACGA | |
| | | GTCCCAAATAGGACGAAACGCGC | |
| 319 | RA319 | GCGCGTCCTGGATTCGCGGAAACGCGTACAGAgaatacca | 0.9 |
| | | gcatcgtcttgatgcccttggaagtcTCTCCAGCTGACGA | |
| | | GTCCCAAATAGGACGAAACGCGC | |
| 320 | RA320 | GCGCGTCCTGGATTCGCGGAAACGCGTACATCCAGCTGAC | 0.9 |
| | | GAGTagtgataccagcatcgtcttgatgcccttggcagca | |
| | | ctCCCAAATAGGACGAAACGCGC | |
| 321 | RA321 | GCGCGTCCTGGATTCGCGGAAACGCGTACATCCAGCTagt | 0.9 |
| | | gataccagcatcgtcttgatgcccttggcagcactGACGA | |
| | | GTCCCAAATAGGACGAAACGCGC | |
| 322 | RA322 | GCGCGTCCTGGATTCGCGGAgaataccagcatcgtcttga | 0.9 |
| | | tgcccttggaagtcTAAACGCGTACATCCAGCTGACGAGT | |
| | | CCCAAATAGGACGAAACGCGC | |
| 323 | RA323 | GCGCGTCCTGGATTCGCGCggaataccagcatcgtcttga | 0.9 |
| | | tgcccttggaagtccGGAAACGCGTACATCCAGCTGACGA | |
| | | GTCCCAAATAGGACGAAACGCGC | |
| 324 | RA324 | GCGCGTCCTGGATTCGCGGAAACCggaataccagcatcgt | 0.9 |
| | | cttgatgcccttggaagtccGGCGTACATCCAGCTGACGA | |
| | | GTCCCAAATAGGACGAAACGCGC | |
| 325 | RA325 | GCGCGTCCTGGATTCGCGGAAACGCGTACATCCAGCTGAC | 0.9 |
| | | GAGTCCCAAATAGGAggaataccagcatcgtcttgatgcc | |
| | | cttggaagtccCaAAACGCGC | |
| 326 | RA326 | GCTGTCACCGGAATACCAGCATCGTCTTGATGCCCTTGGA | 0.9 |
| | | AGTCCGGTCTGATGAGTCCCTTGAGGACGAAACAGC | |
| 327 | RA327 | GCGCGTCCTGGATTCGCGGAAACGCGTagtgataccagca | 0.9 |
| | | tcgtcttgatgcccttggcagcactACATCCAGCTGACGA | |
| | | GTCCCAAATAGGACGAAACGCGC | |
| 328 | RA328 | GCGCGTCCTGGATTCGCGGAAACGCGTACATCCAGCTGAC | 0.9 |
| | | agtgataccagcatcgtcttgatgcccttggcagcactGA | |
| | | GTCCCAAATAGGACGAAACGCGC | |
| 329 | RA329 | GCGCGTCCTGGATTCGCGGAAACGCGTACATCCAGCTGAC | 0.9 |
| | | GAGTCCCAAATAGGACGAAACagtgataccagcatcgtct | |
| | | tgatgcccttggcagcactGCGC | |
| 330 | RA330 | GCGCGTCCTGGGAgaataccagcatcgtcttgatgccctt | 0.9 |
| | | ggaagtcTCATTCGCGGAAACGCGTACATCCAGCTGACGA | |
| | | GTCCCAAATAGGACGAAACGCGC | |
| 331 | RA331 | GCGCGTCCTGggaataccagcatcgtcttgatgcccttgg | 0.9 |
| | | aagtccGATTCGCGGAAACGCGTACATCCAGCTGACGAGT | |
| | | CCCAAATAGGACGAAACGCGC | |
| 332 | RA332 | GCGCGTCCTGGATTCGCGGAAACGCGTACATCCAGCTGAC | 0.9 |
| | | GAGTCggaataccagcatcgtcttgatgcccttggaagtc | |
| | | CCCAAATAGGACGAAACGCGC | |
| 333 | RA333 | GCGCGTCCTGGATTCGCGGAAACGCGTACATCCAGCTGAC | 0.9 |
| | | GAGTCCCAAAgaataccagcatcgtcttgatgcccttgga | |
| | | agtcTATAGGACGAAACGCGC | |
| 334 | RA334 | GCGCGTCCTGGATTCGCGGAAACGCGTACCCggaatacca | 0.9 |
| | | gcatcgtcttgatgcccttggaagtccGGATCCAGCTGAC | |
| | | GAGTCCCAAATAGGACGAAACGCGC | |
| 335 | RA335 | GCGCGTCCTGGATTCGCGGAAACGCGTAggaataccagca | 0.9 |
| | | tcgtcttgatgcccttggaagtccCATCCAGCTGACGAGT | |
| | | CCCAAATAGGACGAAACGCGC | |
| 336 | RA336 | GCGCGTCCTGGATTCGCGGAAagtgataccagcatcgtct | 0.9 |
| | | tgatgcccttggcagcactACGCGTACATCCAGCTGACGA | |
| | | GTCCCAAATAGGACGAAACGCGC | |
| 337 | RA337 | GCGCGTAgaataccagcatcgtcttgatgcccttggaagt | 0.9 |
| | | CTCCTGGATTCGCGGAAACGCGTACATCCAGCTGACGAGT | |
| | | CCCAAATAGGACGAAACGCGC | |
| 338 | RA338 | GCGCGTCCTGGATTCGCGGAAACGAgaataccagcatcgt | 0.9 |
| | | cttgatgcccttggaagtcTCGCGCGTACATCCAGCTGAC | |
| | | GAGTCCCAAATAGGACGAAACGCGC | |
| 339 | RA339 | GCGCGTCCTGGATTggaataccagcatcgtcttgatgccc | 0.9 |
| | | ttggaagtccCGCGGAAACGCGTACATCCAGCTGACGAGT | |
| | | CCCAAATAGGACGAAACGCGC | |
| 340 | RA340 | GCGCagtgataccagcatcgtcttgatgcccttggcagca | 0.9 |
| | | ctGTCCTGGATTCGCGGAAACGCGTACATCCAGCTGACGA | |
| | | GTCCCAAATAGGACGAAACGCGC | |
| 341 | RA341 | GCggaataccagcatcgtcttgatgcccttggaagtccGC | 0.9 |
| | | GTCCTGGATTCGCGGAAACGCGTACATCCAGCTGACGAGT | |
| | | CCCAAATAGGACGAAACGCGC | |
| 342 | RA342 | GCGCGTCCTGGATTCGCGGAAACGCGTACggaataccagc | 0.9 |
| | | atcgtcttgatgcccttggaagtccGCATCCAGCTGACGA | |
| | | GTCCCAAATAGGACGAAACGCGC | |
| 343 | RA343 | GCGCGTCCggaataccagcatcgtcttgatgcccttggaa | 0.9 |
| | | gtccGCTGGATTCGCGGAAACGCGTACATCCAGCTGACGA | |
| | | GTCCCAAATAGGACGAAACGCGC | |
| 344 | RA344 | GCGCGTCCTGGATTCGCGGAAACCCggaataccagcatcg | 0.9 |
| | | tcttgatgcccttggaagtccGGGCGTACATCCAGCTGAC | |
| | | GAGTCCCAAATAGGACGAAACGCGC | |
| 345 | RA345 | GCGCGTCCTGGATTCGCGGAAACGCGTACATCCAGCTGAC | 0.9 |
| | | GAGTCCCAAATAGGACGAAACGCGAgaataccagcatcgt | |
| | | cttgatgcccttggaagtcTC | |
| 346 | RA346 | GCGCGTCCTGGATTCGCGGAAACGCGTACATCCAGCTgga | 0.9 |
| | | ataccagcatcgtcttgatgcccttggaagtccGACGAGT | |
| | | CCCAAATAGGACGAAACGCGC | |
| 347 | RA347 | GCGCGTCCTGGATTCGCGGAAACGCAgaataccagcatcg | 0.9 |
| | | tcttgatgcccttggaagtcTGTACATCCAGCTGACGAGT | |
| | | CCCAAATAGGACGAAACGCGC | |
| 348 | RA348 | GCGCGTCCTGGATTCGCGGAAACGCGTACATCCAGCTGAC | 0.9 |
| | | GACGAgaataccagcatcgtcttgatgcccttggaagtcT | |
| | | CGGTCCCAAATAGGACGAAACGCGC | |
| 349 | RA349 | GCGCGTCCTGGATTCGCGGAAACGCGTACATCCAGCTGAC | 0.9 |
| | | GAGGAgaataccagcatcgtcttgatgcccttggaagtcT | |
| | | CTCCCAAATAGGACGAAACGCGC | |
| 350 | RA350 | GCGCAgaataccagcatcgtcttgatgcccttggaagtcT | 0.9 |
| | | GTCCTGGATTCGCGGAAACGCGTACATCCAGCTGACGAGT | |
| | | CCCAAATAGGACGAAACGCGC | |
| 351 | RA351 | GCGCGTCCTGGATTCGCGGAAACGCGTACATCCAGCGAga | 0.9 |
| | | ataccagcatcgtcttgatgcccttggaagtcTCTGACGA | |
| | | GTCCCAAATAGGACGAAACGCGC | |
| 352 | RA352 | GCGCGTCCTGGATTCGCGGAAACGCGTACATCCAGCTGAC | 0.9 |
| | | GAGTCCCAAATAGGACGAAACGCGCGAgaataccagcatc | |
| | | gtcttgatgcccttggaagtcTCGC | |
| 353 | RA353 | GCGCGTCCTGGATTCGCGGAAACGCGTACATCCAGCTGAC | 0.9 |
| | | GAGTCCCAAATAGGACGAAACGCCggaataccagcatcgt | |
| | | cttgatgcccttggaagtccGGCGC | |
| 354 | RA354 | GCGCGTCCTGGATTCGCGGAAACGCGTACATCCAGCTGAC | 0.9 |
| | | GAGTCCCAAATAGGagtgataccagcatcgtcttgatgcc | |
| | | cttggcagcactACGAAACGCGC | |
| 355 | RA355 | GCGCGTCCTGGATTCGCGGAAACGCGTACATCGAgaatac | 0.9 |
| | | cagcatcgtcttgatgcccttggaagtcTCCAGCTGACGA | |
| | | GTCCCAAATAGGACGAAACGCGC | |
| 356 | RA356 | GCGCGTCCTGGATTCGCGGAAACGCGTACATCCAGCTGAC | 0.8 |
| | | GAGTCCCAAATAGGACGAAAGAgaataccagcatcgtctt | |
| | | gatgcccttggaagtcTCCGCGC | |
| 357 | RA357 | GCGCGTCCTGGATTCGCGGAAACGCGTACATCCAGCTGAC | 0.8 |
| | | GAGTCCCAAATAGGACGggaataccagcatcgtcttgatg | |
| | | cccttggaagtccAAACGCGC | |
| 358 | RA358 | GCGCGTCCTGGATTCGCGGAAACGCGTACATCCAGCTGAC | 0.8 |
| | | GAGCggaataccagcatcgtcttgatgcccttggaagtcc | |
| | | GTCCCAAATAGGACGAAACGCGC | |
| 359 | RA359 | GCGCGTCCTGGATAgaataccagcatcgtcttgatgccct | 0.8 |
| | | tggaagtcTTCGCGGAAACGCGTACATCCAGCTGACGAGT | |
| | | CCCAAATAGGACGAAACGCGC | |
| 360 | RA360 | GCAgaataccagcatcgtcttgatgcccttggaagtcTGC | 0.8 |
| | | GTCCTGGATTCGCGGAAACGCGTACATCCAGCTGACGAGT | |
| | | CCCAAATAGGACGAAACGCGC | |
| 361 | RA361 | GCGCGTCCTGGATTCGCGGAAACGCGTACATCCAGCTGAC | 0.8 |
| | | GAGTCCCAAATAGGACGAAACGCCCggaataccagcatcg | |
| | | tcttgatgcccttggaagtccGGGC | |
| 362 | RA362 | GCGCGTCCTGGAggaataccagcatcgtcttgatgccctt | 0.8 |
| | | ggaagtccTTCGCGGAAACGCGTACATCCAGCTGACGAGT | |
| | | CCCAAATAGGACGAAACGCGC | |
| 363 | RA363 | GCGCGTCCTGGATTCGCGGAAACGCGTACATCCAGCTGAC | 0.8 |
| | | GAGTCCCACggaataccagcatcgtcttgatgcccttgga | |
| | | agtccGAATAGGACGAAACGCGC | |
| 364 | RA364 | GCGCGTCCTGGATTCGCGGAAACGCGTggaataccagcat | 0.8 |
| | | cgtcttgatgcccttggaagtccACATCCAGCTGACGAGT | |
| | | CCCAAATAGGACGAAACGCGC | |
| 365 | RA365 | GCGAgaataccagcatcgtcttgatgcccttggaagtcTC | 0.8 |
| | | GTCCTGGATTCGCGGAAACGCGTACATCCAGCTGACGAGT | |
| | | CCCAAATAGGACGAAACGCGC | |
| 366 | RA366 | GCGCGTCCTGGATTCGCGGAAGAgaataccagcatcgtct | 0.8 |
| | | tgatgcccttggaagtcTCACGCGTACATCCAGCTGACGA | |
| | | GTCCCAAATAGGACGAAACGCGC | |
| 367 | RA367 | GCGCGTCCTGGATggaataccagcatcgtcttgatgccct | 0.8 |
| | | tggaagtccTCGCGGAAACGCGTACATCCAGCTGACGAGT | |
| | | CCCAAATAGGACGAAACGCGC | |
| 368 | RA368 | GCGCGTCCTGGATTCGCGGAAACGCGTACATCCAGCTGAC | 0.8 |
| | | GAGTCCCGAgaataccagcatcgtcttgatgcccttggaa | |
| | | gtcTCGCAAATAGGACGAAACGCGC | |
| 369 | RA369 | GCGCGTCCTGGATTCggaataccagcatcgtcttgatgcc | 0.8 |
| | | cttggaagtccGCGCGGAAACGCGTACATCCAGCTGACGA | |
| | | GTCCCAAATAGGACGAAACGCGC | |
| 370 | RA370 | GCGCGTCCTGGATTCGCGGAAACGCGTACATCCAGCTGAC | 0.8 |
| | | GAGTCCCAAATAGGACGAAACGggaataccagcatcgtct | |
| | | tgatgcccttggaagtccCGC | |
| 371 | RA371 | GCGCGTCCTGGATTCGCGGAAACGCGTACATCCAGCTGAC | 0.8 |
| | | GAGTCCCAAAAgaataccagcatcgtcttgatgcccttgg | |
| | | aagtcTTAGGACGAAACGCGC | |
| 372 | RA372 | GCGCGTCCTGGATTCGCGGAAACGCGTACATCCAGCTGAC | 0.8 |
| | | GAGTCCCAAACGAgaataccagcatcgtcttgatgccctt | |
| | | ggaagtcTCGTAGGACGAAACGCGC | |
| 373 | RA373 | GCGCGTCCTGGATTCGCGGAAACGCGTACATCCAGCTGAC | 0.8 |
| | | GAGTCCCAagtgataccagcatcgtcttgatgcccttggc | |
| | | agcactAATAGGACGAAACGCGC | |
| 374 | RA374 | GCGCGTCCTGGATTCGCGGAAACGCGTACATCCAGCTGCC | 0.8 |
| | | ggaataccagcatcgtcttgatgcccttggaagtccGGAC | |
| | | GAGTCCCAAATAGGACGAAACGCGC | |
| 375 | RA375 | GCGCGTCCTGGATTCGCGGAAACGCGTACATCCAGCTCCg | 0.8 |
| | | gaataccagcatcgtcttgatgcccttggaagtccGGGAC | |
| | | GAGTCCCAAATAGGACGAAACGCGC | |
| 376 | RA376 | GCGCGTCCTGGATTCGCGGAAACGCGTACATCCAGCTGAA | 0.8 |
| | | gaataccagcatcgtcttgatgcccttggaagtcTCGAGT | |
| | | CCCAAATAGGACGAAACGCGC | |
| 377 | RA377 | GCGCGTCCTGGATTCGCGGAAACGCGTACATCCAGCTGAC | 0.8 |
| | | GAGTCCCAAATAGGACGGAgaataccagcatcgtcttgat | |
| | | gcccttggaagtcTCAAACGCGC | |
| 378 | RA378 | GCGCGTCCTGGATTCGCGGAAACGCGTACATCCCggaata | 0.8 |
| | | ccagcatcgtcttgatgcccttggaagtccGGCAGCTGAC | |
| | | GAGTCCCAAATAGGACGAAACGCGC | |
| 379 | RA379 | GCGCGTCCTGGATTCGCGGAAACGCggaataccagcatcg | 0.8 |
| | | tcttgatgcccttggaagtccGTACATCCAGCTGACGAGT | |
| | | CCCAAATAGGACGAAACGCGC | |
| 380 | RA380 | GCGCGTCCTGGATTCGCGGAAACGCCGAgaataccagcat | 0.8 |
| | | cgtcttgatgcccttggaagtcTCGGTACATCCAGCTGAC | |
| | | GAGTCCCAAATAGGACGAAACGCGC | |
| 381 | RA381 | GCGCGTCCTGGATTCGCGGAAACGCGTACATCCAGCTGAC | 0.8 |
| | | GAGTCCCAAATAGGACGAAACGCGCCCggaataccagcat | |
| | | cgtcttgatgcccttggaagtccGG | |
| 382 | RA382 | GCGCGTCCTGGATTCGCGGAAACGCGTACATCCAGCTGAC | 0.8 |
| | | GCCggaataccagcatcgtcttgatgcccttggaagtccG | |
| | | GAGTCCCAAATAGGACGAAACGCGC | |
| 383 | RA383 | CggaataccagcatcgtcttgatgcccttggaagtccGGC | 0.8 |
| | | GCGTCCTGGATTCGCGGAAACGCGTACATCCAGCTGACGA | |
| | | GTCCCAAATAGGACGAAACGCGC | |
| 384 | RA384 | GCGCGTCCTGGATTCGCGGAAACGCGTACATCCAGCTGAC | 0.8 |
| | | GAGTCGAgaataccagcatcgtcttgatgcccttggaagt | |
| | | CTCCCAAATAGGACGAAACGCGC | |
| 385 | RA385 | GCGCGTCCTGGATCCggaataccagcatcgtcttgatgcc | 0.8 |
| | | cttggaagtccGGTCGCGGAAACGCGTACATCCAGCTGAC | |
| | | GAGTCCCAAATAGGACGAAACGCGC | |
| 386 | RA386 | GCGCGTCCTGGATTCGCGGAAACGCGTACATCCAGCTGAC | 0.8 |
| | | GggaataccagcatcgtcttgatgcccttggaagtccAGT | |
| | | CCCAAATAGGACGAAACGCGC | |
| 387 | RA387 | GCGCGTCCTGGATTCGCGGAAACGCGCCggaataccagca | 0.8 |
| | | tcgtcttgatgcccttggaagtccGGTACATCCAGCTGAC | |
| | | GAGTCCCAAATAGGACGAAACGCGC | |
| 388 | RA388 | GCGCGTCCTGagtgataccagcatcgtcttgatgcccttg | 0.8 |
| | | gcagcactGATTCGCGGAAACGCGTACATCCAGCTGACGA | |
| | | GTCCCAAATAGGACGAAACGCGC | |
| 389 | RA389 | GCGCGTCCTGGATTCGCGGAAACGCGTACCggaataccag | 0.8 |
| | | catcgtcttgatgcccttggaagtccGATCCAGCTGACGA | |
| | | GTCCCAAATAGGACGAAACGCGC | |
| 390 | RA390 | GCGCGTCCggaataccagcatcgtcttgatgcccttggaa | 0.7 |
| | | gtccGGCCTGGATTCGCGGAAACGCGTACATCCAGCTGAC | |
| | | GAGTCCCAAATAGGACGAAACGCGC | |
| 391 | RA391 | GCGCGTCCTGGATTCGCGGAAACGCGTACATCCAGCTGAC | 0.7 |
| | | GAGTCCCAGAgaataccagcatcgtcttgatgcccttgga | |
| | | agtcTCAATAGGACGAAACGCGC | |
| 392 | RA392 | GCGCGTCCTGGATTCGCGGACggaataccagcatcgtctt | 0.7 |
| | | gatgcccttggaagtccGAACGCGTACATCCAGCTGACGA | |
| | | GTCCCAAATAGGACGAAACGCGC | |
| 393 | RA393 | GCGCGTCCTGGATTCGCGGAAACGCGTACATCCAGCTGAC | 0.7 |
| | | GAGTCCCAAATAGGCggaataccagcatcgtcttgatgcc | |
| | | cttggaagtccGACGAAACGCGC | |
| 394 | RA394 | GCGCGTCCTGGATTCGCAgaataccagcatcgtcttgatg | 0.7 |
| | | cccttggaagtcTGGAAACGCGTACATCCAGCTGACGAGT | |
| | | CCCAAATAGGACGAAACGCGC | |
| 395 | RA395 | GCGCGTCCTGGATTCGCGGAAACGCGTACATCCAGCTGAC | 0.7 |
| | | GAGTCCCAAATAGGAgaataccagcatcgtcttgatgccc | |
| | | ttggaagtcTCGACGAAACGCGC | |
| 396 | RA396 | GCGCGTCCTGGATTCGCGGAAACGCGTACATCCAGCTGCg | 0.7 |
| | | gaataccagcatcgtcttgatgcccttggaagtccGACGA | |
| | | GTCCCAAATAGGACGAAACGCGC | |
| 397 | RA397 | GCGCGTCCTGGATTCGCGGAAACGCGTACATCCAGCTGAC | 0.7 |
| | | GAGTCCCAAATAGGACGAAACGCGCCggaataccagcatc | |
| | | gtcttgatgcccttggaagtccG | |
| 398 | RA398 | GCGCGTCCTGGATTCGCGGACGAgaataccagcatcgtct | 0.7 |
| | | tgatgcccttggaagtcTCGAACGCGTACATCCAGCTGAC | |
| | | GAGTCCCAAATAGGACGAAACGCGC | |
| 399 | RA399 | GCGCGTCCTGGATTCGCGGAAACGCGTACATCCACGAgaa | 0.7 |
| | | taccagcatcgtcttgatgcccttggaagtcTCGGCTGAC | |
| | | GAGTCCCAAATAGGACGAAACGCGC | |
| 400 | RA400 | GCGCGTCCTGGATTCGCGGAACCggaataccagcatcgtc | 0.7 |
| | | ttgatgcccttggaagtccGGACGCGTACATCCAGCTGAC | |
| | | GAGTCCCAAATAGGACGAAACGCGC | |
| 401 | RA401 | GCGCGTCCTGGATTCGCGGAAACGCGCggaataccagcat | 0.7 |
| | | cgtcttgatgcccttggaagtccGTACATCCAGCTGACGA | |
| | | GTCCCAAATAGGACGAAACGCGC | |
| 402 | RA402 | GCGCGTCCTGGATTCGAgaataccagcatcgtcttgatgc | 0.7 |
| | | ccttggaagtcTCGCGCGGAAACGCGTACATCCAGCTGAC | |
| | | GAGTCCCAAATAGGACGAAACGCGC | |
| 403 | RA403 | GCGCGTCCTCggaataccagcatcgtcttgatgcccttgg | 0.7 |
| | | aagtccGGGATTCGCGGAAACGCGTACATCCAGCTGACGA | |
| | | GTCCCAAATAGGACGAAACGCGC | |
| 404 | RA404 | GCGCGTCCTGGATTCGCGGAAggaataccagcatcgtctt | 0.7 |
| | | gatgcccttggaagtccACGCGTACATCCAGCTGACGAGT | |
| | | CCCAAATAGGACGAAACGCGC | |
| 405 | RA405 | GCGCGTCCTGGATTCGCGGAAACGCGTACGAgaataccag | 0.7 |
| | | catcgtcttgatgcccttggaagtcTCGCATCCAGCTGAC | |
| | | GAGTCCCAAATAGGACGAAACGCGC | |
| 406 | RA406 | GCGCGTCCTGGATTCGCGGAAACGCGTACATCCAGCTGAC | 0.7 |
| | | GAGTCCCCggaataccagcatcgtcttgatgcccttggaa | |
| | | gtccGAAATAGGACGAAACGCGC | |
| 407 | RA407 | GCGCGTCCTGGATTCGCGGAAACGCGTACATCCAGCTGAC | 0.7 |
| | | GAGTCCCAAATAGGACggaataccagcatcgtcttgatgc | |
| | | ccttggaagtccGAAACGCGC | |
| 408 | RA408 | GCGCGTCCTGGATTCGCGGAAACGCGggaataccagcatc | 0.7 |
| | | gtcttgatgcccttggaagtccTACATCCAGCTGACGAGT | |
| | | CCCAAATAGGACGAAACGCGC | |
| 409 | RA409 | GCGCGTCCTGGATTCGCCGAgaataccagcatcgtcttga | 0.7 |
| | | tgcccttggaagtcTCGGGAAACGCGTACATCCAGCTGAC | |
| | | GAGTCCCAAATAGGACGAAACGCGC | |
| 410 | RA410 | GCGCGTCCTGGATTCGCGGAAACGCGTACATCCAGCTGAC | 0.7 |
| | | GAGTCCCagtgataccagcatcgtcttgatgcccttggca | |
| | | gcactAAATAGGACGAAACGCGC | |
| 411 | RA411 | GCGCGTCCTGGATTCGCGGAAACGCGTACATCCAGCTGAC | 0.7 |
| | | GAGTCCCAAATAGCGAgaataccagcatcgtcttgatgcc | |
| | | cttggaagtcTCGGACGAAACGCGC | |
| 412 | RA412 | GCGCGTCCTGGATTCGCGGagtgataccagcatcgtcttg | 0.7 |
| | | atgcccttggcagcactAAACGCGTACATCCAGCTGACGA | |
| | | GTCCCAAATAGGACGAAACGCGC | |
| 413 | RA413 | GCGCGTCCTGGATTCGCGGAAACGCGTAgaataccagcat | 0.7 |
| | | cgtcttgatgcccttggaagtcTACATCCAGCTGACGAGT | |
| | | CCCAAATAGGACGAAACGCGC | |
| 414 | RA414 | GCGCGTCCTGGATTCGCGGAAACGCGTACATCCAGCTGAC | 0.7 |
| | | GAGTCCCCCggaataccagcatcgtcttgatgcccttgga | |
| | | agtccGGAAATAGGACGAAACGCGC | |
| 415 | RA415 | GCGCGTCCTGGATTCGCGGACCggaataccagcatcgtct | 0.7 |
| | | tgatgcccttggaagtccGGAACGCGTACATCCAGCTGAC | |
| | | GAGTCCCAAATAGGACGAAACGCGC | |
| 416 | RA416 | GCGCGTCCTGGACggaataccagcatcgtcttgatgccct | 0.7 |
| | | tggaagtccGTTCGCGGAAACGCGTACATCCAGCTGACGA | |
| | | GTCCCAAATAGGACGAAACGCGC | |
| 417 | RA417 | GCGCGTCCTGGATTCGCGGAAACGCGTACATCCAGCTGAC | 0.7 |
| | | GAGTCCCAAATCggaataccagcatcgtcttgatgccctt | |
| | | ggaagtccGAGGACGAAACGCGC | |
| 418 | RA418 | GCGCGTCCTGGATTCGCGGAAACGCGTACATCGAgaatac | 0.7 |
| | | cagcatcgtcttgatgcccttggaagtcTCGCCAGCTGAC | |
| | | GAGTCCCAAATAGGACGAAACGCGC | |
| 419 | RA419 | GCGCGTCCTGGATTCGCGGAAACGCGTACATCCAGCTGAC | 0.7 |
| | | GAGTCCCAAATAGGACGAAAgaataccagcatcgtcttga | |
| | | tgcccttggaagtcTACGCGC | |
| 420 | RA420 | GCGCGTCCTGGATTCGCGGAAACGGAgaataccagcatcg | 0.6 |
| | | tcttgatgcccttggaagtcTCCGTACATCCAGCTGACGA | |
| | | GTCCCAAATAGGACGAAACGCGC | |
| 421 | RA421 | GCGCGTCCTGGATTCGCGGAAAGAgaataccagcatcgtc | 0.6 |
| | | ttgatgcccttggaagtcTCCGCGTACATCCAGCTGACGA | |
| | | GTCCCAAATAGGACGAAACGCGC | |
| 422 | RA422 | GCGCGTCCTGGATTCGCGGAAACGCGTACATCCAGCTGAC | 0.6 |
| | | GAGTCCCAAATAGGACGACggaataccagcatcgtcttga | |
| | | tgcccttggaagtccGAACGCGC | |
| 423 | RA423 | GCGCGTCCTGGATTCGCGGAAACGCGTACATCCAGCTGAC | 0.6 |
| | | GAGCCggaataccagcatcgtcttgatgcccttggaagtc | |
| | | CGGTCCCAAATAGGACGAAACGCGC | |
| 424 | RA424 | GCGCGTCCTGGATTCGCGGAAACGCGTACATCCAGCTGCG | 0.6 |
| | | AgaataccagcatcgtcttgatgcccttggaagtcTCGAC | |
| | | GAGTCCCAAATAGGACGAAACGCGC | |
| 425 | RA425 | GCGCGTCCTGGATTCGCGGAAACGCGTACAggaataccag | 0.6 |
| | | catcgtcttgatgcccttggaagtccTCCAGCTGACGAGT | |
| | | CCCAAATAGGACGAAACGCGC | |
| 426 | RA426 | GCGCGTCCTGGATTCGCGGAAACGCGTACATCCAGCTGAC | 0.6 |
| | | GAGTCCCAAATAGGACGCCggaataccagcatcgtcttga | |
| | | tgcccttggaagtccGGAAACGCGC | |
| 427 | RA427 | GCGCGTCCTGCCggaataccagcatcgtcttgatgccctt | 0.6 |
| | | ggaagtccGGGATTCGCGGAAACGCGTACATCCAGCTGAC | |
| | | GAGTCCCAAATAGGACGAAACGCGC | |
| 428 | RA428 | GCGCGTCCTGGATTCGCGGAAACGCGTACATCCAGCTGAC | 0.6 |
| | | GAGTCCGAgaataccagcatcgtcttgatgcccttggaag | |
| | | tcTCCAAATAGGACGAAACGCGC | |
| 429 | RA429 | GCGCGTCCTGGATTCGCGGggaataccagcatcgtcttga | 0.6 |
| | | tgcccttggaagtccAAACGCGTACATCCAGCTGACGAGT | |
| | | CCCAAATAGGACGAAACGCGC | |
| 430 | RA430 | GCGCGTCCTGGATTCGCGGAAACGCGTACATCCAGCTGAG | 0.6 |
| | | AgaataccagcatcgtcttgatgcccttggaagtcTCCGA | |
| | | GTCCCAAATAGGACGAAACGCGC | |
| 431 | RA431 | GCGCGTCCTGGATTCGCGGAAACGCGTACATCCACggaat | 0.6 |
| | | accagcatcgtcttgatgcccttggaagtccGGCTGACGA | |
| | | GTCCCAAATAGGACGAAACGCGC | |
| 432 | RA432 | GCGCGTCCTGGATTCGCGGAAACGCGTACATCCAGCTGAC | 0.6 |
| | | GAGTCCCACCggaataccagcatcgtcttgatgcccttgg | |
| | | aagtccGGAATAGGACGAAACGCGC | |
| 433 | RA433 | GCGCGTCCTGGATTCGCGGAAACGCGTACATCCAGCTGAC | 0.6 |
| | | GAGTCCCAAATAGGAagtgataccagcatcgtcttgatgc | |
| | | ccttggcagcactCGAAACGCGC | |
| 434 | RA434 | GCGCGTCCAgaataccagcatcgtcttgatgcccttggaa | 0.6 |
| | | gtcTTGGATTCGCGGAAACGCGTACATCCAGCTGACGAGT | |
| | | CCCAAATAGGACGAAACGCGC | |
| 435 | RA435 | GCGCGTCCTGGATTCGCGGAAACGCGTACATCCAGCTGAC | 0.6 |
| | | GAGTCCCAAACggaataccagcatcgtcttgatgcccttg | |
| | | gaagtccGTAGGACGAAACGCGC | |
| 436 | RA436 | GCGCGTCCTGGATTCGCGGAAACGCGTACATCCAGCggaa | 0.6 |
| | | taccagcatcgtcttgatgcccttggaagtccGCTGACGA | |
| | | GTCCCAAATAGGACGAAACGCGC | |
| 437 | RA437 | GCGCGTCCTGGATTCGCGGAAACGCGTACATCCAGCTGAC | 0.6 |
| | | GAGTCCCAAgaataccagcatcgtcttgatgcccttggaa | |
| | | gtcTAATAGGACGAAACGCGC | |
| 438 | RA438 | GCGCGTCCTGGATTCGCGGAAACGCGTACATCCAGCTGAC | 0.6 |
| | | GAGTCCCAAATAGGACGCggaataccagcatcgtcttgat | |
| | | gcccttggaagtccGAAACGCGC | |
| 439 | RA439 | GCGCGTGAgaataccagcatcgtcttgatgcccttggaag | 0.6 |
| | | tcTCCCTGGATTCGCGGAAACGCGTACATCCAGCTGACGA | |
| | | GTCCCAAATAGGACGAAACGCGC | |
| 440 | RA440 | GCGCGTCCTGGATTCGCGGAAACGCGTACATCCAGCTGAC | 0.6 |
| | | GAGTCCCAAATAGGACGAAACGCCggaataccagcatcgt | |
| | | cttgatgcccttggaagtccGGC | |
| 441 | RA441 | GCGCCCggaataccagcatcgtcttgatgcccttggaagt | 0.6 |
| | | CCGGGTCCTGGATTCGCGGAAACGCGTACATCCAGCTGAC | |
| | | GAGTCCCAAATAGGACGAAACGCGC | |
| 442 | RA442 | GCGCGTCCTGGATTCGCGGAAACGCGTACATCCggaatac | 0.6 |
| | | cagcatcgtcttgatgcccttggaagtccGCAGCTGACGA | |
| | | GTCCCAAATAGGACGAAACGCGC | |
| 443 | RA443 | GCGCGTCCTGGATTCGCGGAAACGCGTACATCCAGCTGAC | 0.6 |
| | | GAGTCCCAAATAGGACggaataccagcatcgtcttgatgc | |
| | | ccttggaagtccGCGAAACGCGC | |
| 444 | RA444 | GCGCGTCCCggaataccagcatcgtcttgatgcccttgga | 0.6 |
| | | agtccGGCTGGATTCGCGGAAACGCGTACATCCAGCTGAC | |
| | | GAGTCCCAAATAGGACGAAACGCGC | |
| 445 | RA445 | GCGCGTCGAgaataccagcatcgtcttgatgcccttggaa | 0.5 |
| | | gtcTCCTGGATTCGCGGAAACGCGTACATCCAGCTGACGA | |
| | | GTCCCAAATAGGACGAAACGCGC | |
| 446 | RA446 | GCGCGTCCTGGATTCCggaataccagcatcgtcttgatgc | 0.5 |
| | | ccttggaagtccGGCGGAAACGCGTACATCCAGCTGACGA | |
| | | GTCCCAAATAGGACGAAACGCGC | |
| 447 | RA447 | GCGCGTCCTGGATTCGCGGAAACGCGTACATCCAGCTGAC | 0.5 |
| | | GAGTCCggaataccagcatcgtcttgatgcccttggaagt | |
| | | CCCAAATAGGACGAAACGCGC | |
| 448 | RA448 | GCGCGCggaataccagcatcgtcttgatgcccttggaagt | 0.5 |
| | | CCGTCCTGGATTCGCGGAAACGCGTACATCCAGCTGACGA | |
| | | GTCCCAAATAGGACGAAACGCGC | |
| 449 | RA449 | GCGCGTCCTGGATTCGCGGAAACGCGTACATCCAGCTGAC | 0.5 |
| | | GAGTCCCAAATAGGACGAAACGCGagtgataccagcatcg | |
| | | tcttgatgcccttggcagcactC | |
| 450 | RA450 | GCGCGTCCTGGATTCGCGGAAACGCGTACATCCAGCTGAC | 0.5 |
| | | GAGTCCCAAATAGGACGAAACCCggaataccagcatcgtc | |
| | | ttgatgcccttggaagtccGGGCGC | |
| 451 | RA451 | GCGCGTCCTGGATTCGCGGAAgaataccagcatcgtcttg | 0.5 |
| | | atgcccttggaagtcTAACGCGTACATCCAGCTGACGAGT | |
| | | CCCAAATAGGACGAAACGCGC | |
| 452 | RA452 | GCGCGTCCTGGAgaataccagcatcgtcttgatgcccttg | 0.5 |
| | | gaagtcTATTCGCGGAAACGCGTACATCCAGCTGACGAGT | |
| | | CCCAAATAGGACGAAACGCGC | |
| 453 | RA453 | GCGCGTCCTGGATTCGCGGAAACGCGTACATCCAGCTGAC | 0.5 |
| | | GAGTCCCAAATAGGACGAAACAgaataccagcatcgtctt | |
| | | gatgcccttggaagtcTGCGC | |
| 454 | RA454 | GCGCGTCCTGGATTCGCGGAAACGCGTACATCCAGCTGAC | 0.5 |
| | | GAGTCCAgaataccagcatcgtcttgatgcccttggaagt | |
| | | CTCAAATAGGACGAAACGCGC | |
| 455 | RA455 | GCGCGTCCTGGATTCGCGGAAACGCGTACATCCAGCTGAC | 0.5 |
| | | GAGTCCCCggaataccagcatcgtcttgatgcccttggaa | |
| | | gtccGGCAAATAGGACGAAACGCGC | |
| 456 | RA456 | GCGCGTCCTGGATTCGCGGAAACGCGTACATCCAGCTGAC | 0.5 |
| | | GAGTCCCAAATACCggaataccagcatcgtcttgatgccc | |
| | | ttggaagtccGGGGACGAAACGCGC | |
| 457 | RA457 | GCGCGTCCTGGATTCGCGGAAACGCGTACATCCAGCTGAC | 0.5 |
| | | GAGTCCCAAATAGGACGAagtgataccagcatcgtcttga | |
| | | tgcccttggcagcactAACGCGC | |
| 458 | RA458 | GCGCGTCCTGGATTCGCGGAAACGCGTACATCCAGCTGAC | 0.5 |
| | | GAGTCCCAAATAGGACGAACCggaataccagcatcgtctt | |
| | | gatgcccttggaagtccGGACGCGC | |
| 459 | RA459 | GCGCGTCCTGGATTCGCGGAAACGCGTACATCCAGCTGAC | 0.5 |
| | | GAGTCCCAAATAGGACGAAggaataccagcatcgtcttga | |
| | | tgcccttggaagtccACGCGC | |
| 460 | RA460 | GCGCGTCCTGGATTCGCagtgataccagcatcgtcttgat | 0.5 |
| | | gcccttggcagcactGGAAACGCGTACATCCAGCTGACGA | |
| | | GTCCCAAATAGGACGAAACGCGC | |
| 461 | RA461 | GCGCGTCCTGGATTCGCGGAAACGCGTACATCCAGGAgaa | 0.5 |
| | | taccagcatcgtcttgatgcccttggaagtcTCCTGACGA | |
| | | GTCCCAAATAGGACGAAACGCGC | |
| 462 | RA462 | GCGCGTCCTGGATTCGCGCCggaataccagcatcgtcttg | 0.4 |
| | | atgcccttggaagtccGGGAAACGCGTACATCCAGCTGAC | |
| | | GAGTCCCAAATAGGACGAAACGCGC | |
| 463 | RA463 | GCGCGTCCTGGATTCGCGGAAACGCGTACATCCAGCTGAC | 0.4 |
| | | GAGTCGAgaataccagcatcgtcttgatgcccttggaagt | |
| | | CTCGCCCAAATAGGACGAAACGCGC | |
| 464 | RA464 | GCGCGTCCTGGagtgataccagcatcgtcttgatgccctt | 0.4 |
| | | ggcagcactATTCGCGGAAACGCGTACATCCAGCTGACGA | |
| | | GTCCCAAATAGGACGAAACGCGC | |
| 465 | RA465 | GCGCGTCCTGGATTCGCGGAAACGCGTACATCCAGCTGAC | 0.4 |
| | | CGAgaataccagcatcgtcttgatgcccttggaagtcTCG | |
| | | GAGTCCCAAATAGGACGAAACGCGC | |
| 466 | RA466 | GCGCGTCCTGGATTCGCGGAACggaataccagcatcgtct | 0.4 |
| | | tgatgcccttggaagtccGACGCGTACATCCAGCTGACGA | |
| | | GTCCCAAATAGGACGAAACGCGC | |
| 467 | RA467 | GCGCGTCCTGGATTCGCGGAAACGAgaataccagcatcgt | 0.4 |
| | | cttgatgcccttggaagtcTCGTACATCCAGCTGACGAGT | |
| | | CCCAAATAGGACGAAACGCGC | |
| 468 | RA468 | GCGCGTCCTGGATTCGCGGAAACGCGTACATCCAGCCGAg | 0.4 |
| | | aataccagcatcgtcttgatgcccttggaagtcTCGIGAC | |
| | | GAGTCCCAAATAGGACGAAACGCGC | |
| 469 | RA469 | GCGAgaataccagcatcgtcttgatgcccttggaagtcTC | 0.4 |
| | | GCGCGTCCTGGATTCGCGGAAACGCGTACATCCAGCTGAC | |
| | | GAGTCCCAAATAGGACGAAACGCGC | |
| 470 | RA470 | GCGCGTCCTGGATTCGCGGAAACCGAgaataccagcatcg | 0.4 |
| | | tcttgatgcccttggaagtcTCGGCGTACATCCAGCTGAC | |
| | | GAGTCCCAAATAGGACGAAACGCGC | |
| 471 | RA471 | GCGCGTCCTGGATTCGCGGAAACGCGTACATCCAGCTGAC | 0.4 |
| | | GAGTCCCAAATAGGACCGAgaataccagcatcgtcttgat | |
| | | gcccttggaagtcTCGGAAACGCGC | |
| 472 | RA472 | GCGCGTCCTGGATTCGCGGAAACGCGTACATCCAGCTGAC | 0.3 |
| | | GAGTCCCAAATAGAgaataccagcatcgtcttgatgccct | |
| | | tggaagtcTCGGACGAAACGCGC | |
| 473 | RA473 | GCGCGTCCTGGATTCGCGGAAACGCGTACATCCAGCTGAC | 0.3 |
| | | GAGTCCCAAATagtgataccagcatcgtcttgatgccctt | |
| | | ggcagcactAGGACGAAACGCGC | |
| 474 | RA474 | GCGCGTCCTGGATTCGCGGAAACGCGTACATCCAGCTGAC | 0.3 |
| | | GAGTCCCAAATAGGACGAAACGCGCggaataccagcatcg | |
| | | tcttgatgcccttggaagtccGC | |
| 475 | RA475 | GCGCGTCCTGGATTCGCGGAAACGCGTACATCCAGCTGAC | 0.3 |
| | | GAGTCCCAAATAGGACGAAACGCGAgaataccagcatcgt | |
| | | cttgatgcccttggaagtcTCGC | |
| 476 | RA476 | GCGCGTCCTGGATTCGCGGAAACGCGTACATCCAGCTGAC | 0.2 |
| | | GAGTCCCAAATAGGACGAAAAgaataccagcatcgtcttg | |
| | | atgcccttggaagtcTCGCGC | |

^{α}Upper-case letters indicate the ribozyme sequence while lower-case letters indicate the aptamer sequence.
βFold change in RNA expression between the ON state and the OFF state.

### Example 3: Validation of Selected Aptazymes

The theophylline inducible aptazymes in Table 4 were tested using a dual fluorescent protein (FP) expression system.

TABLE 4
Aptazyme sequences

| SEQ | | |
|---|---|---|
| ID | | |
| NO | ID | DNA Sequence |
| 475 | BFRA328 | GCGCGTCCTGGATTCGCGGAAACGCGTACATCCAGC |
| | | TGACGAGTCCCAAATAGGACGAAACGCGAGAATACC |
| | | AGCATCGTCTTGATGCCCTTGGAAGTCTCGC |
| 478 | C11 | GCTGTCACCGGAATACCAGCATCGTCTTGATGCCCT |
| | | TGGAAGTCCGGTCTGATGAGTCCCATAAGGACGAAA |
| | | CAGC |
| 210 | RA210 | GCTGTCACCGGAATACCAGCATCGTCTTGATGCCCT |
| | | TGGAAGTCCGGTCTGATGAGTCCAAAAAGGACGAAA |
| | | CAGC |

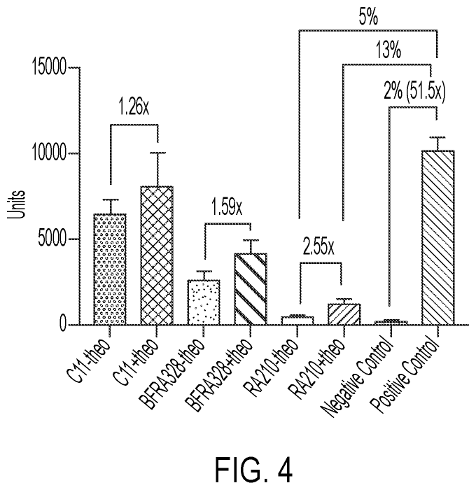

After synthesizing the aptazyme sequences, each of the aptazyme sequences were cloned into the end of a GFP coding sequence of a plasmid that also constitutively expressed red FP (RFP) as a positive marker for transfection. The plasmids were transfected into HEK293-T cells, and the transfected cells were treated with (“ON”) or without (“OFF”) theophylline, as described in Example 1. Green FP (GFP) expression was measured as relative units 24 hours post-transfection (FIG. 4). The positive control used was cells transfected with analogous dual FP plasmid that did not contain any aptazyme. The negative control used was cells transfected with analogous dual FP plasmid containing only the ribozyme sequence, and represented the maximal “OFF” state possible for the ribozyme.

As shown in FIG. 4, the fold-change in GFP expression in transfected cells cultured in the presence of theophylline over GFP expression in transfected cells cultured in the absence of theophylline for the aptazymes C11, BFRA328, and RA210, was 1.26×, 1.59×, and 2.55×, respectively.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

We claim:

1. A polynucleotide comprising a transgene operably linked to a ligand responsive ribozyme, wherein the ligand responsive ribozyme comprises a nucleic acid sequence having at least 85% sequence identity to the sequence set forth in any one of SEQ ID NOs: 1-50.

2. The polynucleotide of claim 1, wherein the ligand responsive ribozyme is inserted into a 3′ untranslated region (UTR) and/or 5′ UTR of the transgene.

3. The polynucleotide of claim 1 or 2, wherein the ligand responsive ribozyme is responsive to theophylline or a derivative thereof.

4. The polynucleotide of claim 3, wherein the theophylline derivative is aminophylline or dyphylline.

5. The polynucleotide of any one of claims 1-4, wherein the transgene encodes one or more polypeptides.

6. The polynucleotide of any one of claims 1-4, wherein the transgene encodes a miRNA, shRNA, siRNA, antisense RNA, gRNA, antagomir, miRNA sponge, RNA aptazyme, RNA aptamer, lncRNA, ribozyme or mRNA.

7. The polynucleotide of any one of claims 1-6, wherein the transgene is operably linked to a transcriptional regulatory element.

8. A vector comprising the polynucleotide of any one of claims 1-7.

9. The vector of claim 8, which is a plasmid, a viral vector, or a DNA minimal vector.

10. The vector of claim 8 or 9, which is an expression vector.

11. The vector of claim 9 or 10, wherein the viral vector is selected from the group consisting of adenoviral vector, adeno-associated virus (AAV) vector, and lentiviral vector.

12. The vector of any one of claims 8-11, which is AAV vector.

13. A ligand responsive ribozyme comprising a nucleic acid sequence having at least 85% identity to the sequence set forth in any one of SEQ ID NOs: 1-50.

14. A recombinant AAV (rAAV) genome comprising the polynucleotide of any one of claims 1-7.

15. The rAAV genome of claim 14, wherein the rAAV genome further comprises a 5′ inverted terminal repeat (5′ ITR) sequence, and a 3′ inverted terminal repeat (3′ ITR) sequence.

16. The rAAV genome of claim 14 or 15, wherein the rAAV genome is a single stranded rAAV genome.

17. The rAAV genome of claim 14 or 15, wherein the rAAV genome is a self-complementary rAAV genome.

18. A recombinant cell comprising the polynucleotide of any one of claims 1-7, the vector of any one of claims 8-12, the ribozyme of claim 13, or the rAAV genome of any one of claims 14-17.

19. A recombinant adeno-associated virus (rAAV) comprising:
(a) a capsid comprising an AAV capsid protein; and
(b) the rAAV genome of any one of claims 14-17.

20. The rAAV of claim 19, wherein the AAV capsid protein is derived from a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein, or an engineered variant thereof.

21. The polynucleotide of any one of claims 1-7, the vector of any one of claims 8-12, or the rAAV of claim 19 or 20, for use in medicine, for use as therapy, or for use as a medicament.

22. A packaging system for preparation of an rAAV, wherein the packaging system comprises:
(a) a first nucleotide sequence encoding one or more AAV Rep proteins;
(b) a second nucleotide sequence encoding an AAV capsid protein; and
(c) a third nucleotide sequence comprising the rAAV genome sequence of any one of claims 14-17.

23. The packaging system of claim 22, wherein the packaging system comprises a first vector comprising the first nucleotide sequence and the second nucleotide sequence, and a second vector comprising the third nucleotide sequence.

24. The packaging system of claim 23, further comprising a fourth nucleotide sequence comprising one or more helper virus genes, optionally wherein the fourth nucleotide sequence is comprised within a third vector.

25. The packaging system of claim 24, wherein the fourth nucleotide sequence comprises one or more genes from a virus selected from the group consisting of adenovirus, herpesvirus, vaccinia virus, and cytomegalovirus (CMV).

26. The packaging system of any one of claims 23-25, wherein the first vector, second vector, and/or the third vector is a plasmid.

27. A method for recombinant preparation of an rAAV, the method comprising introducing the packaging system of any one of claims 23-26 into a cell under conditions whereby the rAAV is produced.

28. A method comprising introducing into a cell the polynucleotide of any one of claims 1-7, the vector of any one of claims 8-12, or the rAAV genome of any one of claims 14-17; or transducing a cell with the rAAV of claim 19 or 20.

29. A method of modulating the expression of a transgene comprising:
(a) introducing into a cell the polynucleotide of any one of claims 1-7, the vector of any one of claims 8-12, or the rAAV genome of any one of claims 14-17; or transducing a cell with the rAAV of claim 19 or 20; and
(b) contacting the cell of (a) with an effective amount of theophylline or a derivative thereof.

30. The method of claim 29, wherein contacting the cell of (a) with an effective amount of the theophylline or the derivative thereof increases expression of the transgene.

31. The method of claim 30, wherein the theophylline derivative is aminophylline or dyphylline.

32. The method of any one of claims 29-31, wherein the cell is in a subject and the polynucleotide, vector, or rAAV is administered to the subject.

33. The method of any one of claims 29-32, wherein the cell is a muscle cell and/or a liver cell.

34. The method of claim 32 or 33, wherein the polynucleotide, vector, or rAAV is administered to the subject intravenously, intraperitoneally, subcutaneously, intramuscularly, intrathecally, or intradermally.

35. A method of modulating the expression of a transgene in a subject, comprising administering theophylline or a derivative thereof to the subject at an amount effect to induce expression of the transgene, wherein the transgene is comprised within the polynucleotide of any one of claims 1-7, the vector of any one of claims 8-12, or the rAAV genome of any one of claims 14-17.

36. The method of claim 35, wherein the transgene further comprises a suicide gene.

37. The method of claim 36, wherein the suicide gene is Herpes Simplex Virus Thymidine Kinase (HSV-TK) or inducible Caspase 9 (iCasp9).

38. The method of claim 36 or 37, further comprising administering an agent to the subject at an amount effective to ablate the recombinant cell.

39. The method of claim 38, wherein the agent is ganciclovir or AP20187.

40. A method of modulating the expression of a transgene in a subject, comprising administering theophylline or a derivative thereof to the subject at an amount effect to induce expression of the transgene, wherein the transgene is comprised within the recombinant cell of claim 18, and the recombinant cell has been administered to the subject.

41. The method of claim 40, wherein the recombinant cell is an immune cell, optionally an immune cell autologous to the subject.

42. The method of claim 41, wherein the immune cell is a T cell, NK cell, NKT cell, or precursor thereof.

43. The method of any one of claims 40-42, wherein the polynucleotide is integrated into a genomic locus of the recombinant cell.

44. The method of claim 43, wherein the genomic locus is a safe harbor locus.

45. The method of any one of claims 40-44, wherein the transgene encodes an exogenous T cell receptor (TCR) and/or chimeric antigen receptor (CAR).

46. The method of any one of claims 40-45, wherein the transgene further comprises a suicide gene.

47. The method of claim 46, wherein the suicide gene is Herpes Simplex Virus Thymidine Kinase (HSV-TK) or inducible Caspase 9 (iCasp9).

48. The method of claim 46 or 47, further comprising administering an agent to the subject at an amount effective to ablate the recombinant cell.

49. The method of claim 48, wherein the agent is ganciclovir or AP20187.

50. The method of any one of claims 32-49, wherein the subject is selected from the group consisting of a human subject, a canine subject, a feline subject, and an equine subject.
